SEQUENCE LISTING (83 sequences)

SEQ ID NO: 1 - protein, 11 aa, Artificial
  CDRL1 of Dom30h-440-81/86, Dom30h-440-53, Dom30h-440-54, Dom30h-440-55, Dom30h-440-56, Dom30h-440-57, Dom30h-440-58, Dom30h-440-60, Dom30h-440-63, Dom30h-440-64 and Dom30h-440-65
RASRPIRNWLD

SEQ ID NO: 2 - protein, 7 aa, Artificial
  CDRL1 of Dom30h-440-81/86
RNWLDWY

SEQ ID NO: 3 - protein, 5 aa, Artificial
  CDRL1 of Dom30h-440-81/86
RNWLD

SEQ ID NO: 4 - protein, 7 aa, Artificial
  CDRL2 of Dom30h-440-81/86, Dom30h-440-53, Dom30h-440-54, Dom30h-440-55, Dom30h-440-56, Dom30h-440-57, Dom30h-440-58, Dom30h-440-60, Dom30h-440-63, Dom30h-440-64 and Dom30h-440-65
GASHLQS

SEQ ID NO: 5 - protein, 10 aa, Artificial
  CDRL2 of Dom30h-440-81/86
LLIWGASHLQ

SEQ ID NO: 6 - protein, 6 aa, Artificial
  CDRL2 of Dom30h-440-81/86
GASHLQ

SEQ ID NO: 7 - protein, 9 aa, Artificial
  CDRL3 of Dom30h-440-81/86 and Dom30h-440-55
VQIGEDPVT

SEQ ID NO: 8 - protein, 8 aa, Artificial
  CDRL3 of Dom30h-440-81/86
VQIGEDPV

SEQ ID NO: 9 - protein, 107 aa, Artificial
  Dom30h-440-81/86 amino acid sequence
DIQMTQSPSSLSASVGDRVTITCRASRPIRNWLDWYQQKPGKAPKLLIWGASHLQSGVPSRFSGSGSGTDFTLTISSLQPEDFATYYCVQIGEDPVTFGQGTKVEIK

SEQ ID NO: 10 - DNA, 321 bp, Artificial
  Non-codon optimised DNA sequence (Dom30h-440-81)
gacatccagatgacccagtctccatcctccctgtctgcatctgtaggagaccgtgtcaccatcacttgccgggcaagtcggcccattcggaattggttagattggtaccagcagaaaccagggaaagcccctaagctcctgatctggggggcgtcccacttgcaaagtggggtcccatcacgtttcagtggcagtggatctgggacagatttcactctcaccatcagcagtctgcaacctgaagattttgctacgtactactgtgtgcagattggggaggatcctgtgacgttcggccaagggaccaaggtggaaatcaaa

SEQ ID NO: 11 - DNA, 321 bp, Artificial
  Codon-optimised DNA sequence (Dom30h-440-86)
gatatccagatgacccagtctccgtcttccctgtctgcgtccgttggtgaccgtgtaaccatcacttgtcgtgcaagccgtccgatccgtaactggctggattggtaccagcagaaaccgggtaaagcgccgaaactgctgatctggggtgcttctcacctgcagtctggtgttccgtcccgtttctctggctctggtagcggtaccgacttcaccctgactatctctagcctgcagccggaagacttcgcgacctactactgcgttcagatcggtgaagacccggttaccttcggtcagggcaccaaagtagaaatcaaa

SEQ ID NO: 12 - protein, 107 aa, Artificial
  Dom30h-440-87/93 amino acid sequence
DIQMTQSPSSLSASVGDRVTITCRASRPIRNWLDWYQQKPGKAPELLIWGASHLQSGVPSRFSGSGSGTDFTLTISSLQPEDFATYYCVQIGEDPVTFGQGTKVEIK

SEQ ID NO: 13 - DNA, 321 bp, Artificial
  Non-codon optimised DNA sequence (Dom30h-440-87)
gatatccagatgacccagtctccgtcttccctgtctgcgtccgttggtgaccgtgtaaccatcacttgtcgtgcaagccgtccgatccgtaactggctggattggtaccagcagaaaccgggtaaagcgccggaactgctgatctggggtgcttctcacctgcagtctggtgttccgtcccgtttctctggctctggtagcggtaccgacttcaccctgactatctctagcctgcagccggaagacttcgcgacctactactgcgttcagatcggtgaagacccggttaccttcggtcagggcaccaaagtagaaatcaaa

SEQ ID NO: 14 - DNA, 321 bp, Artificial
  Codon-optimised DNA sequence (Dom30h-440-93)
gacatccagatgacccagtctccatcctccctgtctgcatctgtaggagaccgtgtcaccatcacttgccgggcaagtcggcccattcggaattggttagattggtaccagcagaaaccagggaaagcccctgagctcctgatctggggggcgtcccacttgcaaagtggggtcccatcacgtttcagtggcagtggatctgggacagatttcactctcaccatcagcagtctgcaacctgaagattttgctacgtactactgtgtgcagattggggaggatcctgtgacgttcggccaagggaccaaggtggaaatcaaa

SEQ ID NO: 15 - protein, 9 aa, Artificial
  CDRL3 of Dom30h-440-53
LQVGEDPVT

SEQ ID NO: 16 - protein, 9 aa, Artificial
  CDRL3 of Dom30h-440-54
WQLAFDPTT

SEQ ID NO: 17 - protein, 9 aa, Artificial
  CDRL3 of Dom30h-440-56 and Dom30h-440-65
MQIGEDPVT

SEQ ID NO: 18 - protein, 9 aa, Artificial
  CDRL3 of Dom30h-440-57
MQIGDDPVT

SEQ ID NO: 19 - protein, 9 aa, Artificial
  LQIADDPVT
LQIADDPVT

SEQ ID NO: 20 - protein, 9 aa, Artificial
  CDRL3 of Dom30h-440-60
MQIGSDPVT

SEQ ID NO: 21 - protein, 9 aa, Artificial
  CDRL3 of Dom30h-440-63
MQIGSDPVT

SEQ ID NO: 22 - protein, 9 aa, Artificial
  CDRL3 of Dom30h-440-64
LQIGEDPVT

SEQ ID NO: 23 - protein, 108 aa, Artificial
  Dom30h-440-53 amino acid sequence
DIQMTQSPSSLSASVGDRVTITCRASRPIRNWLDWYQQKPGKAPKLLIWGASHLQSGVPSRFSGSGSGTDFTLTISSLQPEDFATYYCLQVGEDPVTFGQGTKVEIKR

SEQ ID NO: 24 - protein, 108 aa, Artificial
  Dom30h-440-54 amino acid sequence
DIQMTQSPSSLSASVGDRVTITCRASRPIRNWLDWYQQKPGKAPKLLIWGASHLQSGVPSRFSGSGSGTDFTLTISSLQPEDFATYYCWQLAFDPTTFGQGTKVEIKR

SEQ ID NO: 25 - protein, 108 aa, Artificial
  Dom30h-440-55 amino acid sequence
DIQMTQSPSSLSASVGDRVTITCRASRPIRNWLDWYQQKPGKAPKLLIWGASHLQSGVPSRFSGSGSGTDFTLTISSLQPEDFATYYCVQIGEDPVTFGQGTKVEIKR

SEQ ID NO: 26 - protein, 108 aa, Artificial
  Dom30h-440-56 amino acid sequence
DIQMTQSPSSLSASVGDRVTITCRASRPIRNWLDWYQQKPGKAPKLLIWGASHLQSGVPSRFSGSGSGTDFTLTVSSLQPEDFATYYCMQIGEDPVTFGQGTKVEIKR

SEQ ID NO: 27 - protein, 108 aa, Artificial
  Dom30h-440-57 amino acid sequence
DIQMTQSPSSLSASVGDRVTITCRASRPIRNWLDWYQQKPGKAPELLIWGASHLQSGVPSRFSGSGSGTDFTLTISSLQPEDFATYYCMQIGDDPVTFGQGTKVEIKR

SEQ ID NO: 28 - protein, 108 aa, Artificial
  Dom30h-440-58 amino acid sequence
DIQMTQSPSSLSASVGDRVTITCRASRPIRNWLDWYQQKPGKAPKLLIWGASHLQSGVPSRFSGSGSGTDFTLTISSLQPEDFATYYCLQIADDPVTFGQGTKVEIKR

SEQ ID NO: 29 - protein, 108 aa, Artificial
  Dom30h-440-60 amino acid sequence
DIQMTQSPSSLSASVGDRVTITCRASRPIRNWLDWYQQKPGKAPKLLIWGASHLQSGVPSRFSGSGSGTDFTLTISSLQPEDFATYYCIQFGEDPVTFGQGTKVEIKR

SEQ ID NO: 30 - protein, 108 aa, Artificial
  Dom30h-440-63 amino acid sequence
DIQMTQSPSSLSASVGDRVTITCRASRPIRNWLDWYQQKPGKAPKLLIWGASHLQSGVPSRFSGSGSGTDFTLTISSLQPEDFATYYCMQIGSDPVTFGQGTKVEIKR

SEQ ID NO: 31 - protein, 108 aa, Artificial
  Dom30h-440-64 amino acid sequence
DIQMTQSPSSLSASVGDRVTITCRASRPIRNWLDWYQQKPGKAPKLLIWGASHLQSGVPSRFSGSGSGTDFTLTISSLQPEDFATYYCLQIGEDPVTFGQGTKVEIKR

SEQ ID NO: 32 - protein, 108 aa, Artificial
  Dom30h-440-65 amino acid sequence
DIQMTQSPSSLSASVGDRVTITCRASRPIRNWLDWYQQKPGKAPKLLIWGASHLQSGVPSRFSGSGSGTDFTLTISSLQPEDFATYYCMQIGEDPVTFGQGTKVEIKR

SEQ ID NO: 33 - protein, 107 aa, Artificial
  Dom30h-440-88/92 amino acid sequence
DIQMTQSPSSLSASVGDRVTITCRASRPIRNWLDWYQQKPGKAPELLIWGASHLQSGVPSRFSGSGSGTDFTLTISSLQPEDFATYYCMQIGDDPVTFGQGTKVEIK

SEQ ID NO: 34 - protein, 108 aa, Artificial; identical to SEQ ID NO: 25
  Dom30h-440-89/94 amino acid sequence

SEQ ID NO: 35 - protein, 108 aa, Artificial
  Dom30h-440-90/95 amino acid sequence
DIQMTQSPSSLSASVGDRVTITCRASRPIRNWLDWYQQKPGKAPELLIWGASHLQSGVPSRFSGSGSGTDFTLTISSLQPEDFATYYCVQIGEDPVTFGQGTKVEIKR

SEQ ID NO: 36 - protein, 108 aa, Artificial; identical to SEQ ID NO: 27
  Dom30h-440-91/96 amino acid sequence

SEQ ID NO: 37 - protein, 17 aa, Artificial
  Peptide sequence containing long “epitope” for TSLP binding protein
YMSGTKSTEFNNTVSCS

SEQ ID NO: 38 - protein, 12 aa, Artificial
  Peptide sequence containing short “epitope” for TSLP binding protein
KSTEFNNTVSCS

SEQ ID NO: 39 - protein, 8 aa, Artificial
  Peptide containing residues 0-7 of full length human TSLP
MYDFTNCD

SEQ ID NO: 40 - protein, 9 aa, Artificial
  Peptide containing residues 0-8 of full length human TSLP
MYDFTNCDF

SEQ ID NO: 41 - protein, 17 aa, Artificial
  Peptide containing residues 0-16 of full length human TSLP
MYDFTNCDFEKIKAAYL

SEQ ID NO: 42 - protein, 13 aa, Artificial
  Peptide containing residues 1-13 of full length human TSLP
YDFTNCDFEKIKA

SEQ ID NO: 43 - protein, 8 aa, Artificial
  Peptide containing residues 8-15 of full length human TSLP
FEKIKAAY

SEQ ID NO: 44 - protein, 9 aa, Artificial
  Peptide containing residues 8-16 of full length human TSLP
FEKIKAAYL

SEQ ID NO: 45 - protein, 7 aa, Artificial
  Peptide containing residues 9-15 of full length human TSLP
EKIKAAY

SEQ ID NO: 46 - protein, 8 aa, Artificial
  Peptide containing residues 9-16 of full length human TSLP
EKIKAAYL

SEQ ID NO: 47 - protein, 10 aa, Artificial
  Peptide containing residues 9-18 of full length human TSLP
EKIKAAYLST

SEQ ID NO: 48 - protein, 7 aa, Artificial
  Peptide containing residues 10-16 of full length human TSLP
KIKAAYL

SEQ ID NO: 49 - protein, 9 aa, Artificial
  Peptide containing residues 17-25 of full length human TSLP
STISKDLIT

SEQ ID NO: 50 - protein, 10 aa, Artificial
  Peptide containing residues 17-26 of full length human TSLP
STISKDLITY

SEQ ID NO: 51 - protein, 7 aa, Artificial
  Peptide containing residues 19-25 of full length human TSLP
ISKDLIT

SEQ ID NO: 52 - protein, 8 aa, Artificial
  Peptide containing residues 19-26 of full length human TSLP
ISKDLITY

SEQ ID NO: 53 - protein, 13 aa, Artificial
  Peptide containing residues 23-35 of full length human TSLP
LITYMSGTKSTEF

SEQ ID NO: 54 - protein, 10 aa, Artificial
  Peptide containing residues 26-35 of full length human TSLP
YMSGTKSTEF

SEQ ID NO: 55 - protein, 24 aa, Artificial
  Peptide containing residues 26-49 of full length human TSLP
YMSGTKSTEFNNTVSCSNRPHCLT

SEQ ID NO: 56 - protein, 25 aa, Artificial
  Peptide containing residues 26-50 of full length human TSLP
YMSGTKSTEFNNTVSCSNRPHCLTE

SEQ ID NO: 57 - protein, 9 aa, Artificial
  Peptide containing residues 27-35 of full length human TSLP
MSGTKSTEF

SEQ ID NO: 58 - protein, 16 aa, Artificial
  Peptide containing residues 35-50 of full length human TSLP
FNNTVSCSNRPHCLTE

SEQ ID NO: 59 - protein, 14 aa, Artificial
  Peptide containing residues 36-49 of full length human TSLP
NNTVSCSNRPHCLT

SEQ ID NO: 60 - protein, 15 aa, Artificial
  Peptide containing residues 36-50 of full length human TSLP
NNTVSCSNRPHCLTE

SEQ ID NO: 61 - protein, 9 aa, Artificial
  Peptide containing residues 42-50 of full length human TSLP
SNRPHCLTE

SEQ ID NO: 62 - protein, 12 aa, Artificial
  Peptide containing residues 51-62 of full length human TSLP
IQSLTFNPTAGC

SEQ ID NO: 63 - protein, 9 aa, Artificial
  Peptide containing residues 53-61 of full length human TSLP
SLTFNPTAG

SEQ ID NO: 64 - protein, 9 aa, Artficial Sequence
  Peptide containing residues 54-62 of full length human TSLP
LTFNPTAGC

SEQ ID NO: 65 - protein, 12 aa, Artificial
  Peptide containing residues 54-65 of full length human TSLP
LTFNPTAGCASL

SEQ ID NO: 66 - protein, 11 aa, Artificial
  Peptide containing residues 55-65 of full length human TSLP
TFNPTAGCASL

SEQ ID NO: 67 - protein, 18 aa, Artificial
  Peptide containing residues 60-77 of full length human TSLP
AGCASLAKEMFAMKTKAA

SEQ ID NO: 68 - protein, 9 aa, Artificial
  Peptide containing residues 70-78 of full length human TSLP
FAMKTKAAL

SEQ ID NO: 69 - protein, 10 aa, Artificial
  Peptide containing residues 70-79 of full length human TSLP
FAMKTKAALA

SEQ ID NO: 70 - protein, 9 aa, Artificial
  Peptide containing residues 71-79 of full length human TSLP
AMKTKAALA

SEQ ID NO: 71 - protein, 7 aa, Artificial
  Peptide containing residues 79-85 of full length human TSLP
AIWCPGY

SEQ ID NO: 72 - protein, 8 aa, Artificial
  Peptide containing residues 79-86 of full length human TSLP
AIWCPGYS

SEQ ID NO: 73 - protein, 9 aa, Artificial
  Peptide containing residues 79-87 of full length human TSLP
AIWCPGYSE

SEQ ID NO: 74 - protein, 10 aa, Artificial
  Peptide containing residues 79-88 of full length human TSLP
AIWCPGYSET

SEQ ID NO: 75 - protein, 11 aa, Artificial
  Peptide containing residues 80-90 of full length human TSLP
IWCPGYSETQI

SEQ ID NO: 76 - protein, 7 aa, Artificial
  Peptide containing residues 89-95 of full length human TSLP
QINATQA

SEQ ID NO: 77 - protein, 10 aa, Artificial
  Peptide containing residues 115-124 of full length human TSLP
QLQGLWRRFN

SEQ ID NO: 78 - protein, 9 aa, Artificial
  Peptide containing residues 119-127 of full length human TSLP
LWRRFNRPL

SEQ ID NO: 79 - protein, 18 aa, Artificial
  Peptide containing residues 120-137 of full length human TSLP
WRRFNRPLLKQQHHHHHH

SEQ ID NO: 80 - protein, 131 aa, homo sapiens
YDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 81 - protein, 63 aa, homo sapiens
MFAMKTKAALAIWCPGYSETQINATQAMKKRRKRKVTTNKCLEQVSQLQGLWRRFNRPLLKQQ

SEQ ID NO: 82 - protein, 131 aa, homo sapiens; identical to SEQ ID NO: 80

SEQ ID NO: 83 - protein, 152 aa, homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

(19) United States
(12) Patent Application Publication
(10) Pub. No.: US 2017/0260266 A1
(43) Pub. Date: Sep. 14, 2017

(54) TSLP BINDING PROTEINS

(71) Applicant: GlaxoSmithKline Intellectual Property Development Limited, Brentford (GB)

(72) Inventors: Farheen AHMED, Stevenage (GB); Michelle Anne BARTHOLOMEW, Stevenage (GB); Chun-Wa CHUNG, Stevenage (GB); Pietro DELLA CRISTINA, Stevenage (GB); Caroline J. DIMECH, Stevenage (GB); Peter Joseph MORLEY, Stevenage (GB); Rachana Shailesh SHAH, Stevenage (GB)

(21) Appl. No.: 15/451,896

(22) Filed: Mar. 7, 2017

Related U.S. Application Data
(63) Continuation-in-part of application No. 15/064,745, filed on Mar. 9, 2016.
(60) Provisional application No. 62/131,285, filed on Mar. 11, 2015.

(51) Int. Cl. C07K 16/24

(52) U.S. Cl. CPC C07K 16/244; C07K 2317/33; C07K 2317/565; C07K 2317/92; C07K 2317/76; A61K 2039/505

(57) ABSTRACT

The present disclosure relates to TSLP binding proteins that interact with particular residues of human full length TSLP, or contact particular regions of human full length TSLP. The invention also includes pharmaceutical compositions and medical uses of these TSLP binding proteins.

This application is a continuation-in-part of U.S. application Ser. No. 15/064,745, filed Mar. 9, 2016, which claims the benefit of U.S. Provisional Application No. 62/131,285 filed Mar. 11, 2015, which are incorporated herein in their entireties.

### FIELD OF THE INVENTION

The present disclosure relates to TSLP binding proteins, such as anti-TSLP single variable domains, polynucleotides encoding such TSLP binding proteins, pharmaceutical compositions and kits comprising said TSLP binding proteins and methods of manufacture. The present invention also concerns the use of such TSLP binding proteins in the treatment of diseases associated TSLP signalling, such as asthma.

The present disclosure relates to TSLP binding proteins that interact with particular residues of human full length TSLP, or contact particular regions of human full length TSLP. The invention also includes pharmaceutical compositions and medical uses of these TSLP binding proteins.

### BACKGROUND TO THE INVENTION

Full length thymic stromal lymphopoetin (TSLP) was originally identified as a factor in supernatants from mouse thymic stromal cells which could induce the proliferation of pre-B cells (Friend, et al., Exp Hematol. 22(3):321, 1994). The murine protein was later identified (Sims, et al., J Exp Med. 2000 Sep. 4; 192(5):671-80), closely followed by identification of human TSLP in 2001 by two separate groups (Quentmeier, et al., Leukemia. 2001 August; 15(8):1286-92, Reche, et al. J Immunol. 2001 Jul. 1; 167(1):336-43).

Full length TSLP is a short-chain four α-helical bundle cytokine that induces Signal Transducer and Activator of Transcription (STATS) phosphorylation via the functional TSLP receptor (TSLPR), a heterodimeric receptor complex consisting of the IL-7Rα and the unique TSLPR chain (CRFL2) (Park et al, JEM 192(5):659-682 (2002)). In addition a short isoform of TSLP (sfTSLP) expressed from an alternative transcription start site appears to be expressed in human cells, but does not appear to activate STATS and may serve a different function to full length TSLP (Bjerkan et al., Mucosal immunology 8(1) 49-56 (2015)).

TSLP is most highly produced by epithelial and stromal cells lining the barrier surfaces of the skin, gut, and lungs but is also produced by other cell types implicated in allergic disease (e.g., dendritic cells, mast cells, smooth muscle cells). Production is induced upon exposure to a number of factors including protease allergens (Kouzaki et al, J Immunol. 183(2):1427-34 (2009)), viruses, bacteria, inflammatory mediators, cigarette smoke and environmental particulates (Bleck et al, J Clin Immunol 28(2):147-156 (2008)).

TSLP acts on a broad range of cell types (e.g., dendritic cells, CD4+ T cells, eosinophils, basophils, mast cells and Type 2 innate lymphoid cells (ILC2) (Mjosberg et al, Immunity 37(4):649-59 (2012)) to drive inflammation, and in particular, Type 2 inflammation (characterised by the production of the cytokines IL-5, IL-13 and IL-4. Type 2 inflammation is a feature of asthma and other allergic diseases such as atopic dermatitis and Netherton Syndrome. TSLP has been found to induce fibroblast accumulation and collagen deposition in animals demonstrating an additional role in promoting fibrotic disorders.

A critical role for TSLP in the development and maintenance of allergic disease is supported by pre-clinical animal model data. Mice deficient in TSLP signalling are resistant to the development of asthma (Zhou et al, Nat Immunol 6(10): 1047-1053 (2005)), and neutralisation of TSLP or its receptor with antibodies is efficacious in murine or primate asthma or rhinitis models. For example, blocking TSLP with an anti-TSLPR mAb in a primate asthma model (cynomolgus monkeys naturally sensitised to Ascaris suum antigen) reduced eosinophilia airway resistance and IL-13 levels (Cheng et al, Journal of Allergy and Clinical Immunology 132(2):455-462 (2013)).

TSLP is over-expressed in the epithelium and lamina propria of lungs of asthmatic subjects at both the mRNA and protein level (Ying et al. J Immunol. 181(4):2790-2798 (2008); Shikotra et al. J Allergy Clin Immunol. 129(1):104-111 (2012); Kaur et al. Chest. 142(1):76-85 (2012)), even in patients taking high dose inhaled corticosteroids. Strong supportive data for the importance of TSLP in asthma comes from the efficacy of an anti-TSLP monoclonal antibody (AMG-157/MEDI9929) in an allergen challenge study in mild asthmatics (Gavreau et al, N Engl J Med. 370(22):2102-2110 (2014). After 6 or 12 weeks of treatment (once monthly dosing) with AMG-157 significant effects were observed on early and late phase responses measured by changes in FEV₁, and in blood and sputum eosinophil counts, and FeNO levels

Asthma is a common chronic disease affecting an estimated 300 million people worldwide, and symptoms can be controlled in many patients, using bronchodilators (e.g. β2-aderenergic receptor agonists) and inhaled or oral corticosteroids, depending on the severity of the disease. However, a large number of moderate and severe asthmatics remain symptomatic and inadequately controlled, affecting quality of life and representing a significant healthcare burden. Particularly, many patients with severe asthma may be unresponsive or respond poorly to high doses of steroids.

Omalizumab (Xolair™) is a humanised IgG1 mAb-targeting soluble IgE, and is approved for the treatment of adults and adolescents (12 years of age and above) with moderate to severe persistent asthma who have a positive skin test or in vitro reactivity to a perennial aeroallergen and whose symptoms are inadequately controlled with inhaled corticosteroids. When used as an adjunct to current therapies, it has been proven to reduce exacerbations (Busse, et al. Curr Med Res Opin. 2007 October; 23(10):2379-86). However, omalizumab is not suitable for all asthmatics, its use being restricted to patients satisfying particular defined criteria, such as serum IgE 30-700 IU/ml.

Accordingly, there is considerable need for novel asthma treatments which could be either stand-alone therapies, or be used as add-on therapies, for patients uncontrolled on an existing standard of care therapy.

### SUMMARY OF THE INVENTION

In one embodiment, the present invention provides a TSLP binding protein that comprises CDR1, CDR2 and CDR3 of SEQ ID NO: 9 or a variant of any one or all of these CDRs, wherein the CDR variant has 1, 2, or 3 amino acid modifications; or an amino acid sequence at least 90% identical to the sequence of SEQ ID NO:9.

A CDR variant includes an amino acid sequence modified by at least one amino acid, wherein said modification can be chemical or a partial alteration of the amino acid sequence, which modification permits the variant to retain the biological characteristics of the unmodified sequence. A partial alteration of the CDR amino acid sequence may be by deletion or substitution of one or more amino acids, or by addition or insertion of one or more amino acids, or by a combination thereof. The CDR variant may contain 1, 2 or 3 amino acid substitutions, additions or deletions, in any combination, in the amino acid sequence.

Typically, the modification is a substitution or a conservative substitution, for example, as shown in Table 1 below. In one embodiment, a CDR is modified by the substitution of up to 3 amino acids, for example, 1 or 2 amino acids, for example 1 amino acid. In an embodiment each of the three CDRs is modified, independently of the other two CDRs, by 2, 1 or none amino acid residues.

TABLE 1

| Side chain | Members |
|---|---|
| Hydrophobic | Met, Ala, Val, Leu, Ile |
| Neutral hydrophilic | Cys, Ser, Thr |
| Acidic | Asp, Glu |
| Basic | Asn, Gln, His, Lys, Arg |
| Residues that influence chain | Gly, Pro |
| orientation | |
| Aromatic | Trp, Tyr, Phe |

In certain CDR1 variants, the residue corresponding to residue 28 in SEQ ID NO:9 is Pro, the residue corresponding to residue 30 in SEQ ID NO:9 is Arg, the residue corresponding to residue 31 in SEQ ID NO:9 is Asn, the residue corresponding to residue 32 in SEQ ID NO: 9 is Trp and the residue corresponding to residue 34 in SEQ ID NO:9 is Asp. In certain CDR2 variants, the residue corresponding to residue 50 in SEQ ID NO:9 is Gly, the residue corresponding to residue 53 in SEQ ID NO:9 is His and the residue corresponding to residue 55 in SEQ ID NO:9 is Gln. In more particular CDR2 variants, in addition to the residues identified before, the residue corresponding to residue 46 in SEQ ID NO:9 is Leu. In certain CDR3 variants, the residue corresponding to residue 91 in SEQ ID NO:9 is Ile, Leu, Val or Phe, the residue corresponding to residue 92 in SEQ ID NO:9 is Gly or Ala, the residue corresponding to residue 93 in SEQ ID NO:9 is Glu, Phe, Asp or Ser and the residue corresponding to residue 94 in SEQ ID NO:9 is Asp. In more particular CDR3 variants, the residue corresponding to residue 91 in SEQ ID NO:9 is Ile, Leu or Val.

In one embodiment, CDR3 consists of the sequence X₁GlnX₂X₃X₄AspProX₅Thr, wherein X₁ represents Lys, Trp, Val, Met or Ile, X₂ represents Val, Leu, Ile or Phe, X₃ represents Gly or Ala, X₄ represents Glu, Phe, Asp or Ser, and X₅ represents Val or Thr. More particularly, consists of the sequence X₁GlnX₂X₃X₄AspProX₅Thr, wherein X₁ represents Lys, Trp, Val or Met, X₂ represents Val, Leu or Ile, X₃ represents Gly or Ala, X₄ represents Glu, Phe, Asp or Ser, and X₅ represents Val or Thr.

In a more particular embodiment, the TSLP binding protein comprises CDR1, CDR2 and CDR3 of SEQ ID NO: 9.

In the foregoing embodiments, the CDRs can be defined by any numbering convention, for example the Kabat, Chothia, AbM and Contact conventions. Alternatively, the CDRs may be the minimum binding unit (those residues that form part of the CDR by the Kabat, Chothia, AbM and Contact definitions). The CDR regions for SEQ ID NO. 9, defined by each method are set out in Table 2. The skilled reader would understand that each of CDR1, CDR2 and CDR3 may be defined by a different numbering convention, or that more than one CDR may be defined by the same numbering convention.

TABLE 2

| | CDR1 | CDR2 | CDR3 |
|---|---|---|---|
| Kabat | RASRPIRNWLD | GASHLQS | VQIGEDPVT |
| CDR | (SEQ ID NO: 1) | (SEQ ID NO: 4) | (SEQ ID NO: 7) |
| Chothia | RASRPIRNWLD | GASHLQS | VQIGEDPVT |
| CDR | (SEQ ID NO: 1) | (SEQ ID NO: 4) | (SEQ ID NO: 7) |
| AbM CDR | RASRPIRNWLD | GASHLQS | VQIGEDPVT |
| | (SEQ ID NO: 1) | (SEQ ID NO: 4) | (SEQ ID NO: 7) |
| Contact | RNWLDWY | LLIWGASHLQ | VQIGEDPV |
| CDR | (SEQ ID NO: 2) | (SEQ ID NO: 5) | (SEQ ID NO: 8) |
| Minimum | RNWLD | GASHLQ | VQIGEDPV |
| binding | (SEQ ID NO: 3) | (SEQ ID NO: 6) | (SEQ ID NO: 8) |
| unit | | | |

In certain embodiments of the foregoing binding proteins, all CDRs are defined according to the Kabat numbering convention such that CDR1 consists of the sequence defined as SEQ ID NO: 1 or a variant thereof, CDR2 consists of the sequence defined as SEQ ID NO: 4 or a variant thereof and CDR3 consists of the sequence defined as SEQ ID NO:7 or a variant thereof (wherein the variation permitted is outlined above). In a more particular embodiment, all CDRs are defined according to the Kabat numbering convention such that CDR1 consists of the sequence defined as SEQ ID NO: 1, CDR2 consists of the sequence defined as SEQ ID NO: 4 and CDR3 consists of the sequence defined as SEQ ID NO:7.

In another embodiment, CDR1 and CDR3 are defined according to the Kabat numbering convention such that CDR1 consists of the sequence defined as SEQ ID NO: 1 or a variant thereof, and CDR3 consists of the sequence defined as SEQ ID NO:7 or a variant thereof, and CDR2 is defined according to the Contact numbering system such that it consists of the sequence defined in SEQ ID NO. 5 or a variant thereof. In a more particular embodiment, CDR1 consists of the sequence defined as SEQ ID NO: 1, CDR3 consists of the sequence defined as SEQ ID NO:7 and CDR2 consists of the sequence defined in SEQ ID NO. 5.

In a more particular embodiment, the TSLP binding protein comprises CDR1, CDR2 and CDR3 wherein:

(i) CDR1 is as present in SEQ ID NO:9 or is a variant of this sequence having one or more substitutions selected from: Ile 29 substituted for Val and Leu 33 substituted for Met, Val, Ile or Phe,

(ii) CDR2 is as present in SEQ ID NO:9 or is a variant of this sequence wherein Ala 51 is substituted for Thr, and

(iii) CDR3 is as present in SEQ ID NO:9 or is a variant of this sequence having one or more substitutions selected from: Val 89 substituted for Gln, Ser, Gly, Phe or Leu; Gln 90 substituted for Asn or His; Ile 91 substituted for Val or Phe; Gly 93 is substituted for Ala; Glu 93 substituted for Ser; Asp 94 substituted for Glu; Val 96 substituted for Pro, Tyr, Arg, Ile, Trp or Phe.

In the context of this invention, the term “substituted for”, for example in the phrase “Ile29 substituted for Val” refers to replacement of the first mentioned residue (in this case Ile29) with the second mentioned residue (in this case Val).

In particular embodiments of the foregoing binding proteins, the TSLP binding protein has an IC50 of less than or equal to 5 nM. For example, the invention provides a TSLP binding protein that comprises CDR1, CDR2 and CDR3 of SEQ ID NO: 9 or a variant of any one or all of these CDRs, wherein the CDR variant has 1, 2, or 3 amino acid modifications; or an amino acid sequence at least 90% identical to the sequence of SEQ ID NO:9; which TSLP binding protein has an IC50 of less than or equal to 5 nM.

IC50 may be measured by methods known in the art, for example in the Receptor Binding Assay described in Example 1 or in the Cell Assay described in Example 2 or the Inhibition of TSLP-induced TARC (CCL17) in human whole blood assay described in Example 5. In one embodiment, IC50 is measured by the Cell Assay described in Example 2. One of ordinary skill in the art will appreciate that there is variation in individual values of IC50 calculated from different experiments. In one embodiment, the IC50 value is the mean calculated from at least three experiments. In another embodiment, the IC50 value is the geometric mean calculated from at least three experiments.

In particular embodiments of the foregoing binding proteins, the TSLP binding protein binds to full length human TSLP with a dissociation constant (KD) of less than 2 nM. In one embodiment, the KD value is the mean calculated from at least three experiments.

In particular embodiments of the foregoing binding proteins, the TSLP binding protein exhibits no significant binding to IL-7 (in one embodiment, no binding). Binding affinity may be determined by equilibrium methods (e.g., enzyme-linked immunoabsorbent assay (ELISA) or radioimmunoassay (RIA)), or kinetics (e.g., Biacore™ analysis).

In certain embodiments of the foregoing binding protein, the TSLP binding protein competes for binding to full length human TSLP with a single variable domain of SEQ ID NO:9.

In one embodiment of any of the foregoing binding proteins, the TSLP binding protein is an anti-TSLP single variable domain. In a particular embodiment the single variable domain is a VL domain. In a particular embodiment the VL domain is a V_{K} domain.

In one embodiment where the TSLP binding protein is a V_{K} domain, the residue corresponding to residue 27 in SEQ ID NO:9 is Arg, the residue corresponding to residue 29 in SEQ ID NO: 9 is Ile, the residue corresponding to residue 89 in SEQ ID NO:9 is Val and the residue corresponding to residue 96 in SEQ ID NO: 9 is Val. In one embodiment, the residue corresponding to residue 49 in SEQ ID NO:9 is Trp. In one embodiment, the residue corresponding to residue 36 in SEQ ID NO:9 is Tyr, the residue corresponding to residue 38 in SEQ ID NO:9 is Gln, the residue corresponding to residue 44 in SEQ ID NO:9 is Pro, the residue corresponding to residue 67 in SEQ ID NO:9 is Ser, the residue corresponding to residue 87 in SEQ ID NO:9 is Tyr, the residue corresponding to residue 98 in SEQ ID NO:9 is Phe and the residue corresponding to residue 100 in SEQ ID NO:9 is Gln.

In a more particular embodiment, the anti-TSLP single variable domain consists of the sequence defined as SEQ ID NO. 9 or a variant of SEQ ID NO. 9 that differs in having up to 10 amino acid additions, deletions or substitutions with the proviso that the additions, deletions, or substitutions are not at positions corresponding to residues 28, 30, 31, 32, 34, 50, 53, 55, 91, 92, 93 and 94 of SEQ ID NO. 9. More particularly, the anti-TSLP single variable domain consists of the sequence defined as SEQ ID NO. 9 or a variant of SEQ ID NO. 9 that differs in having up to 10 amino acid additions, deletions or substitutions with the proviso that the additions, deletions, or substitutions are not at positions corresponding to residues 27, 28, 29, 30, 31, 32, 34, 50, 53, 55, 89, 91, 92, 93, 94 and 96 of SEQ ID NO. 9. Even more particularly, the anti-TSLP single variable domain consists of the sequence defined as SEQ ID NO. 9 or a variant of SEQ ID NO. 9 that differs in having up to 10 amino acid additions, deletions or substitutions with the proviso that the additions, deletions, or substitutions are not at positions corresponding to residues 27, 28, 29, 30, 31, 32, 34, 46, 49, 50, 53, 55, 89, 91, 92, 93, 94 and 96 of SEQ ID NO. 9. More particularly, the anti-TSLP single variable domain consists of the sequence defined as SEQ ID NO. 9 or a variant of SEQ ID NO. 9 that differs in having up to 10 amino acid additions, deletions or substitutions with the proviso that the additions, deletions, or substitutions are not at positions corresponding to residues 27, 28, 29, 30, 31, 32, 34, 36, 38, 44, 46, 49, 50, 53, 55, 87, 89, 91, 92, 93, 94, 96, 98 and 100 of SEQ ID NO. 9. More particularly, the anti-TSLP single variable domain consists of the sequence defined as SEQ ID NO. 9 or a variant of SEQ ID NO. 9 that differs in having up to 10 amino acid additions, deletions or substitutions with the proviso that the additions, deletions, or substitutions are not at positions corresponding to residues 27, 28, 29, 30, 31, 32, 34, 36, 38, 44, 46, 49, 50, 53, 55, 67, 87, 89, 91, 92, 93, 94, 96, 98 and 100 of SEQ ID NO. 9. In an even more particular embodiment of the anti-TSLP single variable domain described above, the CDRs are defined as described in any of the above embodiments.

It will be appreciated by one of skill in the art that the variant of SEQ ID NO. 9 may contain 1, 2, 3, 4, 5, 6, 7, 8, 9 or 10 amino acid substitutions, additions or deletions, in any combination. Typically, the modification is a substitution. In one embodiment, the sequences are modified by the substitution of 1, 2, 3, 4, 5, 6, 7, 8, 9 or 10 amino acids. In a more particular embodiment, the modification is a conservative substitution (a substitution of one amino acid residue for another residue in the same group of Table 2).

In another embodiment, the TSLP binding protein (anti-TSLP single variable domain) consists of the sequence defined as SEQ ID NO. 9 or a sequence that has at least 90% sequence identity to the sequence of SEQ ID NO. 9. In more particular embodiments, the percentage identity with SEQ ID NO. 9 is greater than or equal to 90, 91, 92, 93, 94, 95, 96, 97, 98 or 99.

Percent identity between a query amino acid sequence and a subject amino acid sequence is the identities value expressed as a percentage that is calculated by the BLASTP algorithm when a subject amino acid sequence has a 100% query coverage with a query amino acid sequence after a pair wise BLASTP alignment is performed. Such pair-wise BLASTP alignments between a query amino acid sequence and a subject amino acid sequence are performed by using the default settings of the BLASTP algorithm available on the National Center for Biotechnology Institute's website with the filter for low complexity regions turned off.

The percentage identity may be determined across the entire length of the query sequence. Alternatively, the percentage identity may exclude particular residues which are fixed/intact. In one embodiment, residues corresponding to positions 28, 30, 31, 32, 34, 49, 50, 53, 55, 91, 92, 93 and 94 of SEQ ID NO. 9 are fixed. In a more particular embodiment, residues corresponding to positions 27, 28, 29, 30, 31, 32, 34, 50, 53, 55, 89, 91, 92, 93 and 96 of SEQ ID NO. 9 are fixed. Even more particularly, residues corresponding to positions 27, 28, 29, 30, 31, 32, 34, 46, 49, 50, 53, 55, 89, 91, 92, 93, 94 and 96 of SEQ ID NO. 9 are fixed. Even more particularly, residues corresponding to positions 27, 28, 29, 30, 31, 32, 34, 36, 38, 44, 46, 49, 50, 53, 55, 87, 89, 91, 92, 93, 94, 96, 98 and 100 of SEQ ID NO. 9 are fixed. More particularly, residues corresponding to positions 27, 28, 29, 30, 31, 32, 34, 36, 38, 44, 46, 49, 50, 53, 55, 67, 87, 89, 91, 92, 93, 94, 96, 98 and 100 of SEQ ID NO. 9 are fixed. In one embodiment, the percentage identity with SEQ ID NO. 9 excluding fixed positions is greater than or equal to 90, 91, 92, 93, 94, 95, 96, 97, 98 or 99.

In another embodiment, the percentage identity may exclude the CDRs (which may be defined as described in any of the above embodiments) and the residue corresponding to position 49 of SEQ ID NO. 9. More particularly, the percentage identity may exclude the CDRs (which may be defined as described in any of the above embodiments) and residues corresponding to positions 36, 38, 44, 46, 49, 87, 98 and 100 of SEQ ID NO. 9. Even more particularly, the percentage identity may exclude the CDRs (which may be defined as described in any of the above embodiments) and residues corresponding to positions 36, 38, 44, 46, 49, 67, 87, 98 and 100 of SEQ ID NO. 9. In one embodiment, the percentage identity with SEQ ID NO. 9 excluding fixed positions is greater than or equal to 90, 91, 92, 93, 94, 95, 96, 97, 98 or 99.

In one embodiment, the anti-TSLP single variable domain consists of the amino acid sequence defined as SEQ ID NO. 9.

In one embodiment, the anti-TSLP single variable domain comprises an amino acid sequence selected from the group consisting of: SEQ ID NO:12, SEQ ID NO: 23, SEQ ID NO:24, SEQ ID NO:25, SEQ ID NO:26, SEQ ID NO:27, SEQ ID NO: 28, SEQ ID NO: 29, SEQ ID NO: 30, SEQ ID NO:31, SEQ ID NO: 32, SEQ ID NO: 33, SEQ ID NO:34, SEQ ID NO:35 and SEQ ID NO:36. In a more particular embodiment, the amino acid sequence has a C-terminus ending in RT. In an alternative embodiment, the amino acid sequence has a C-terminus that does not end in R.

In one embodiment, the anti-TSLP single variable domain consists of an amino acid sequence selected from the group consisting of: SEQ ID NO:12, SEQ ID NO: 23, SEQ ID NO:24, SEQ ID NO:25, SEQ ID NO:26, SEQ ID NO:27, SEQ ID NO: 28, SEQ ID NO: 29, SEQ ID NO: 30, SEQ ID NO:31, SEQ ID NO: 32, SEQ ID NO: 33, SEQ ID NO:34, SEQ ID NO:35 and SEQ ID NO:36.

In one embodiment, there is provided a polypeptide comprising the anti-TSLP single variable domain described above.

In one embodiment, the present invention provides a TSLP binding protein that comprises the following CDRs: CDR1, CDR2 and CDR3 from SEQ ID NO:9, or a variant of any one or all of these CDRs, wherein the TSLP binding protein binds to TSLP with a dissociation constant (KD) of less that 2 nM or competes for binding to TSLP with a single variable domain of SEQ ID NO:9. For the avoidance of doubt, the TSLP binding protein that competes for binding to TSLP with a single variable domain of SEQ ID NO:9 must also have CDR1, CDR2 and CDR3 from SEQ ID NO:9, or a variant of any one or all of these CDRs. The TSLP used for competition studies is, in one embodiment, full length human TSLP.

In a more particular embodiment, the present invention provides a TSLP binding protein that comprises the following CDRs: CDR1, CDR2 and CDR3 from SEQ ID NO:9, or a variant of any one or all of these CDRs, wherein the TSLP binding protein binds to TSLP with a dissociation constant (KD) of less that 2 nM and competes for binding to TSLP with a single variable domain of SEQ ID NO:9.

In another embodiment, the present invention provides a TSLP binding protein that comprises:

(i) CDR1, according to SEQ ID NO:1 or a variant of SEQ ID NO:1, wherein Ile 29 is substituted for Val; Leu 33 is substituted for Met, Val, Ile or Phe;

(ii) CDR2, according to SEQ ID NO:4 or a variant of SEQ ID NO:4 wherein Ala 51 is substituted for Thr, and

(iii) CDR3, according to SEQ ID NO:7 or a variant of SEQ ID NO:7 wherein Val 89 is substituted for Gln, Ser, Gly, Phe or Leu; Gln 90 is substituted for Asn or His; Ile 91 is substituted for Phe or Val; Glu 93 is substituted for Ser; Val 96 is substituted for Pro, Tyr, Arg, Ile, Trp or Phe; and

wherein the TSLP binding protein is capable of binding to TSLP with a dissociation constant (KD) of less that 2 nM and/or competes for binding to TSLP with a single variable domain of SEQ ID NO:9.

In another embodiment, the present invention provides an anti-TSLP single variable domain comprising an amino acid sequence according to SEQ ID NO:9, having up to 10 amino acid substitutions, deletions or additions, in any combination that binds to TSLP with a dissociation constant (KD) of less than 2 nM and/or competes for binding to TSLP with a single variable domain of SEQ ID NO:9.

In yet another embodiment, the present invention provides an isolated polypeptide comprising an anti-TSLP single variable domain of the disclosure, wherein said isolated polypeptide binds to TSLP.

In another embodiment, the present invention provides nucleic acids encoding a TSLP binding protein, an anti-TSLP single variable domain, or a polypeptide of the disclosure, together with vectors and host cells comprising the same and methods for producing the same.

In yet another embodiment, the present invention provides a pharmaceutical composition comprising a TSLP binding protein, an anti-TSLP single variable domain, or a polypeptide of the disclosure. Such polypeptides or pharmaceutical compositions may be used in treatment of a disease associated with TSLP signalling.

In yet another embodiment, the present invention provides a kit comprising a TSLP binding protein, an anti-TSLP single variable domain, or a polypeptide of the disclosure, and a device for inhaling said TSLP binding protein, an anti-TSLP single variable domain or polypeptide of the disclosure.

Summary of the Aspects of the Invention Relating to TSLP Binding Proteins Binding to a Particular “Epitope”

The domain antibody DOM-30h-440-81/86 having the amino acid sequence set out in SEQ ID NO: 9 has been characterised and shown to have the following desirable properties:
1. Binding to full length human TSLP
2. Cross reactivity with cynomolgus TSLP
3. No significant binding to human IL-7
4. Inhibition of binding of TSLP to TSLP receptor
5. Inhibition of TSLP biological mediated effects (e.g. induction of pSTAT5 or TARC) in cell assays (using recombinant human TSLP, inhibition of pSTAT5 induction has been demonstrated using native TSLP derived from human lung fibroblasts)

As a result of these desirable properties, this domain antibody is believed to be useful in the treatment of diseases associated with TSLP signalling.

Whilst other antagonists of TSLP may result in the inhibition of TSLP biological mediated effects, properties 1-4 arise from the selection of the epitope. For example, cross reactivity reflects the fact that the epitope is in a region of TSLP that is conserved between human and cynomolgus monkey. Advantageously, cross reactivity with cynomolgus monkey permits pre-clinical work to be carried out in this species, facilitating the development of binding proteins having this epitope. The inhibition of binding of TSLP to the TSLP receptor arises because the epitope is in the region of TSLP responsible for receptor binding (see FIG. 7). In view of this, binding proteins targeting this epitope should exhibit reduced internalisation compared to binding proteins that allow TSLP to bind to either IL-7Rα or TSLPR. The lack of binding to IL-7 arises because the epitope on TSLP does not resemble an epitope on IL-7. This selectivity is highly desirable as it avoids interfering with the action of IL-7 on the body (e.g. acting as a growth factor for lymphoid cells). The binding to full length human TSLP and not short form TSLP reflects the fact that the epitope of TSLP is in part in a portion of TSLP not present in the short isoform.

Other TSLP binding proteins contacting the same epitope of the human full length TSLP as DOM30h-440-81/86 with suitable affinity may be expected to have similar biological activity. Such binding proteins would be highly desirable.

Most definitions of the term “epitope” specify that the epitope is the part of the antigen that is in contact with an antibody or fragment thereof (see, for example, Essential Immunology, Sixth Edition, Blackwell Scientific Publishing, 1988, Ed. Roitt, Chapter 4). Other definitions refer to the part of the antigen that is bound by the antibody or fragment. The terms “contact” and “bound” might imply that an epitope should properly only consist of residues that directly interact with the antibody or fragment via non-covalent interactions such as electrostatics (hydrogen bonding, ionic), Van de Waals forces, π-effects and hydrophobic bonds. On such a strict interpretation, an epitope would not include residues that do not interact, but assist in the interaction between antigen and antibody (or fragment). For example, certain residues in the antigen are preferably small (e.g., glycine) to permit the close interaction required to facilitate direct interaction between other residues of the antigen and antibody (or fragment). Similarly, certain residues (e.g., proline) may assist the antigen sequence to adopt the correct conformation to permit binding.

In fact, most of the techniques typically performed in order to identify “epitope” information do not (and cannot) distinguish between interacting residues and residues that assist binding in other ways. The following techniques are commonly used:
1. Binding of antibodies or fragments thereof to peptides derived from the antigen (wherein peptides that exhibit significant binding are considered to contain “the epitope”).
2. Hydrogen deuterium exchange (wherein peptides derived from complexed antigen that are resistant to deuterium incorporation compared to uncomplexed antigen are deemed to contain “the epitope”)
3. Mutagenesis studies (e.g. alanine scanning mutagenesis, wherein mutated positions in the antigen significantly alter binding to the antibody or fragment are deemed to form part of “the epitope”).
   As will be apparent to the skilled person, only techniques with atomic level resolution (e.g. X-ray crystallography, NMR, electron microscopy) are capable of distinguishing between residues that interact and those that otherwise assist binding. However, even though these are the only techniques capable of giving information on the epitope according to a strict definition, it is submitted that the other techniques nonetheless provide useful information on residues/sequences that are in certain ways involved in the binding of antibodies or fragments to their target. Interacting residues identified by high resolution structural analysis are likely more important to binding than the residues identified by other techniques.

The binding of DOM-30h-440-81/86 to human full length TSLP for binding has been investigated by hydrogen deuterium exchange and X-ray crystallography (see examples 14 and 15). Deuterium uptake by TSLP was reduced in the presence of the DOM-30h-440-81/86 in two non-consecutive regions: Tyr26 to Ser42 and Gln89 to Ala95. This data suggests a discontinuous epitope where two linear peptide regions are brought together by the three dimensional structure of TSLP. A discontinuous epitope was also suggested by the X-ray crystallography findings which showed that the residues in TSLP that directly interacted with DOM-30h-440-81/86 were Lys31, Phe35, Arg121 and Arg122 and also showed that the residues which exhibited a ≧60% change in buried surface area were Ser32, Thr33, Asn37, Ser40, Cys41, Ser42, Ser114, Gly118, Arg121, Arg122, Arg125 and Leu128 (see Example 1 and FIG. 2).

It will be apparent that the most N-terminal portion of the discontinuous epitope is consistently identified by the two techniques (with the interacting residues and residues exhibiting a large change in buried surface area falling within the Tyr26-Ser42 region identified by hydrogen deuterium exchange). The C-terminal portion of the discontinuous epitope identified by hydrogen deuterium exchange however, did not contain any of the residues identified as directly interacting with DOM-30h-440-81/86, or indeed any of the residues which exhibited a ≧60% change in buried surface area by X ray crystallography. This partly reflects the fact that fewer peptides containing the C-terminal region were generated in the hydrogen deuterium exchange experiment, but it is also thought to reflect the fact that the C-terminal region may make a smaller contribution to the “epitope”. Example 15 and FIG. 10 show that peptides containing the Tyr26 to Ser42 region were most strongly protected from deuterium uptake. Similarly, the X ray crystallography data presented in Example 14 shows significantly more direct and indirect interactions in the N-terminal portion of the epitope than in the C-terminal portion. Further evidence of this comes from the inability of DOM-30h-440-81/86 to bind to the short isoform of TSLP which lacks the N-terminal portion of the epitope. For this reason, it is believed that the region between Tyr26 and Ser42 contain the residues that interact or are otherwise required for binding. It is also believed that the region between residues 31 and 42 contain the residues that interact or are otherwise required for binding. It is believed that binding proteins capable of binding to linear peptides with these sequences may have similar biological activity to DOM-30h-440-81/86.

Accordingly, in a first aspect the present invention provides a TSLP binding protein that either:

a) interacts with the following residues of full length human TSLP (SEQ ID NO: 80): Lys31 and Phe35; or
b) increases the % buried surface area of residues Ser32, Thr33, Asn37, Ser40, Cys41 and Ser42, when complexed with full length human TSLP, as compared to uncomplexed full length human TSLP; or
c) binds to full length human TSLP having at least one residue selected from: Lys31, Ser32, Thr33, Phe35, Asn36, Asn37, Ser40, Cys41, Ser42, Arg121 and Arg122 mutated exhibiting altered affinity in comparison with full length human TSLP with no mutations; or
d) binds to a peptide having the amino acid sequence set forth in SEQ ID NO: 37; or
e) binds to full length human TSLP and results in peptides derived from full length human TSLP containing part or the whole of the sequence from Tyr26 and Ser42 being more resistant to deuterium incorporation compared to corresponding peptides derived from uncomplexed full length human TSLP.

In one embodiment, said TSLP binding protein does not have the amino acid sequence set forth in SEQ ID NO: 9.

In further embodiments, said TSLP binding protein does not have the amino acid sequence set forth in any of SEQ ID NOs: 12, 23, 24, 25, 26, 27, 28, 29, 30, 31, 32, 33, 34, 35 or 36.

In one embodiment, said TSLP binding protein is not a TSLP-binding protein comprising:
a. CDR1, CDR2 and CDR3 of SEQ ID NO: 9 or a variant of any one or all of these CDRs, wherein the CDR variant has 1, 2, or 3 amino acid modifications; or
b. an amino acid sequence at least 90% identical to the sequence of SEQ ID NO: 9;
   which TSLP binding protein has an IC50 of less than or equal to 5 nM.

In one embodiment, said TSLP binding protein is not a TSLP binding protein that binds an epitope comprising the following residues of full length human TSLP (SEQ ID NO: 80): Tyr15, Lys31, Ser32, Thr33, Phe35, Asn36, Asn37, Ser40, Cys41, Ser42, Ser114, Gln115, Gln117, Gly118, Arg121, Arg122, Arg125, Pro126, Leu128 and Lys 129.

In one embodiment, said TSLP binding protein does not compete for binding to TSLP with a single variable domain of SEQ ID NO:9. In one embodiment, the TSLP used in the competition assay is full length human TSLP.

In further aspects, the present invention provides a TSLP binding protein of the invention for use as a medicament. In another aspect, the invention provides a TSLP binding protein of the invention for use in treating a disease associated with TSLP signalling. The invention also provides a pharmaceutical composition comprising a TSLP binding protein of the invention and optionally one or more pharmaceutically acceptable carriers.

In another aspect, the invention provides a TSLP binding protein that binds a particular epitope. In one embodiment, epitope residues for a particular TSLP binding protein may be identified using the Qt-PISA v2.0.1 software (Protein Interfaces, Complexes and Assemblies; Krissinel and Henrick (2007) as being those residues on full length human TSLP where greater than or equal to 20% of the exposed surface area becomes buried on binding to the TSLP binding protein. Epitope residues may thus comprise the following residues of full length human TSLP: Tyr15, Lys31, Ser32, Thr33, Phe35, Asn36, Asn37, Ser40, Cys41, Ser42, Ser114, Gln115, Gln117, Gly118, Arg121, Arg122, Arg125, Pro126, Leu128 and Lys 129. In another embodiment, epitope residues for a particular TSLP binding protein may be identified using the Qt-PISA v2.0.1 software (Protein Interfaces, Complexes and Assemblies; Krissinel and Henrick (2007) as being those residues on full length human TSLP which exhibit an increase in % buried surface area on binding to the TSLP binding protein. In addition to the residues already identified, epitope residues may thus further comprise the following residues of full length human TSLP: Ser20, Ile24, Glu34, Thr38, Val39, Asn43, His46, Asn124 and Leu127.

In one embodiment, the TSLP binding protein that binds to the epitope described above is an antibody. More particularly, the TSLP binding protein that binds to the above mentioned epitope is a single variable domain. In a more particular embodiment the single variable domain is a VL domain. More particularly, the VL domain is a V_{K} domain. In one embodiment, the single variable domain that binds to the above mentioned epitope is non-naturally occurring.

In particular embodiments, the TSLP binding protein that binds to the epitope described above exhibits no significant binding to IL-7 (in one embodiment, no binding). Binding affinity may be determined by equilibrium methods (e.g., enzyme-linked immunoabsorbent assay (ELISA) or radioimmunoassay (RIA)), or kinetics (e.g., Biacore™ analysis).

### BRIEF DESCRIPTION OF THE FIGURES

FIG. 1 is a flow chart showing the generation, optimisation and characterisation of anti-TSLP dAbs.

FIG. 2 is a graph showing the binding of dAbs to pre-existing human anti-V_{κ} (HAVK) antibodies: DOM-30h-440-87, DOM-30h-440-88, DOM-30h-440-90 and DOM-30h-440-91 (Vk dAbs with −R or + T modifications at the C-terminus) had reduced binding to pre-existing HAVK antibodies compared with DT02-K-044-085 (unmodified native C-terminus).

FIG. 3 is a graph showing that DOM-30h-440-81/86 inhibited binding of recombinant human TSLP (1 ng/ml) and native human TSLP (supernatant from human lung fibroblasts) to the TSLP receptor complex (TSLP Receptor Binding Assay (RBA)).

FIG. 4 is a graph showing the frequency of healthy human donor serum samples that contain pre-existing antibodies to DOM-30h-440-81/86 (—R C-terminus) compared with dAb DT02-K-044-085 (unmodified native C-terminus).

FIG. 5 is a flowchart showing the fermentation process for production of DOM30h-440-81/86 at a 150 L scale.

FIG. 6 shows the downstream purification process for purification of DOM30h-440-81/86 from clarified fermentation broth.

FIG. 7 is an overlay of the X-ray structure of TSLP-DOM-30h-440-81/86 (dark ribbon) with literature complex TSLP/IL7Rα/TSLPR complex (PDB: 4NN7, in lighter grey shades).

FIG. 8A shows the % surface area buried on TSLP-DOM30h-440-81/86 complex formation for individual residues of TSLP (1-55). FIG. 8B shows the % surface area buried on TSLP-DOM30h-440-81/86 complex formation for individual residues of TSLP (56-129).

FIG. 9A shows the % surface area buried on TSLP-DOM30h-440-81/86 complex formation for individual residues of DOM30h-440-81/86 (1-56). FIG. 9B shows the % surface area buried on TSLP-DOM30h-440-81/86 complex formation for individual residues of DOM30h-440-81/86 (57-108).

FIG. 10 is a representative plot showing the fractional difference in deuterium update between uncomplexed full length human TSLP and TSLP-DOM30h-440-81/86 for individual peptides at 30 and 300 seconds after dilution into deuterated buffer.

### DETAILED DESCRIPTION OF THE INVENTION

Aspects of the Invention relating to TSLP binding proteins binding to a particular “epitope”

In a first aspect, the present invention provides a TSLP binding protein that either:

a) interacts with the following residues of full length human TSLP (SEQ ID NO: 80): Lys31 and Phe35; or
b) increases the % buried surface area of residues Ser32, Thr33, Asn37, Ser40, Cys41 and Ser42, when complexed with full length human TSLP, as compared to uncomplexed full length human TSLP; or
c) binds to full length human TSLP having at least one residue selected from: Lys31, Ser32, Thr33, Phe35, Asn36, Asn37, Ser40, Cys41, Ser42, Arg121 and Arg122 mutated exhibiting altered affinity in comparison with full length human TSLP with no mutations; or
d) binds to a peptide having the amino acid sequence set forth in SEQ ID NO: 37; or
e) binds to full length human TSLP and results in peptides derived from full length human TSLP containing part or the whole of the sequence from Tyr26 and Ser42 being more resistant to deuterium incorporation compared to corresponding peptides derived from uncomplexed full length human TSLP.

In one embodiment, the invention provides a TSLP binding protein that interacts with the following residues of full length human TSLP (SEQ ID NO: 80): Lys31 and Phe35. In another embodiment, the TSLP binding protein interacts with the following residues of full length human TSLP (SEQ ID NO: 80): Lys31, Ser32, Thr33, Phe35 and Asn37. In another embodiment, the TSLP binding protein interacts with the following residues of full length human TSLP (SEQ ID NO: 80): Lys31, Ser32, Thr33, Phe35, Asn36, Asn37, Ser40, Cys41 and Ser42. In an alternative embodiment, the invention provides a TSLP binding protein that interacts with the following residues of full length human TSLP (SEQ ID NO: 80): Lys31, Phe35, Arg121 and Arg122.

In the above embodiments, the interaction may be a direct interaction or an indirect interaction via water. In one embodiment, the interaction is a direct interaction. In the context of this invention, a direct interaction is a hydrogen bond between the TSLP binding protein and the named residue(s) of full length human TSLP. Interacting residues may be identified by any technique capable of atomic level resolution. In one embodiment, interacting residues are identified by X-ray crystallography.

In one embodiment, the invention provides a TSLP binding protein that increases the % buried surface area of residues Ser32, Thr33, Asn37, Ser40, Cys41 and Ser42 when complexed with full length human TSLP (SEQ ID NO: 80), as compared to uncomplexed full length human TSLP.

The % buried surface area of a residue is increased if a greater percentage of the surface of the residue is buried in the binding protein-full length human TSLP complex when compared to the % buried in uncomplexed full length human TSLP. In one embodiment, the increase in % buried surface area is greater than or equal to 40%, in another embodiment, the increase in % buried surface area is greater than or equal to 50%, and in an additional embodiment, the increase in % buried surface area is greater than or equal to 60%. The change in percentage buried surface area may be calculated by any suitable technique, for example, comparison of the X ray crystal structures of complexed and uncomplexed TSLP.

In one embodiment, the invention provides a TSLP binding protein that binds to full length human TSLP (SEQ ID NO: 80) having one or more residues of Lys31, Ser32, Thr33, Phe35, Asn36, Asn37, Ser40, Cys41, Ser42 Arg121 and Arg122 mutated exhibiting altered affinity in comparison with full length human TSLP with no mutations. Binding may be assessed by any suitable method, for example, SPR or ELISA. The TSLP may be tagged (e.g. biotinylated) to facilitate the binding assay, but its sequence may not be extended by additional amino acids. The term “altered affinity” refers to the situation where the antibody or fragment exhibits substantially reduced or substantially increased affinity for the mutated version when compared with wild type full length human TSLP. A substantial increase in affinity is where the mean K_{D} for the mutated version measured on the basis of at least three readings is less than or equal to the mean K_{D} for wild type full length human TSLP measured on the basis of at least three readings minus one standard deviation (the larger of the standard deviations for the wild type or mutated version should be used). In one embodiment, a substantial increase in affinity is where the mean K_{D} for the mutated version measured on the basis of at least three readings is less than or equal to the mean K_{D} for wild type full length human TSLP measured on the basis of at least three readings minus two standard deviations (the larger of the standard deviations for the wild type or mutated version should be used). In a further embodiment, a substantial increase in affinity is where the mean K_{D} for the mutated version measured on the basis of at least three readings is less than or equal to the mean K_{D} for wild type full length human TSLP measured on the basis of at least three readings minus three standard deviations (the larger of the standard deviations for the wild type or mutated version should be used). In one embodiment, a substantial increase in affinity is where the mean K_{D} for the mutated version measured on the basis of at least three readings is at least 3 fold less than the mean K_{D} for wild type full length human TSLP measured on the basis of at least three readings. In another embodiment, a substantial increase in affinity is where the mean K_{D} for the mutated version measured on the basis of at least three readings is at least 5 fold less than the mean K_{D} for wild type full length human TSLP measured on the basis of at least three readings. In yet another embodiment, a substantial increase in affinity is where the mean K_{D} for the mutated version measured on the basis of at least three readings is at least 10 fold less than the mean K_{D} for wild type full length human TSLP measured on the basis of at least three readings. A substantial decrease in affinity is where the mean K_{D} for the mutated version measured on the basis of at least three readings is greater than or equal to the mean K_{D} for wild type full length human TSLP measured on the basis of at least three readings plus one standard deviation (the larger of the standard deviations for the wild type or mutated version should be used). In one embodiment, a substantial decrease in affinity is where the mean K_{D} for the mutated version measured on the basis of at least three readings is greater than or equal to the mean K_{D} for wild type full length human TSLP measured on the basis of at least three readings plus two standard deviations (the larger of the standard deviations for the wild type or mutated version should be used). In a further embodiment, a substantial decrease in affinity is where the mean K_{D} for the mutated version measured on the basis of at least three readings is greater than or equal to the mean K_{D} for wild type full length human TSLP measured on the basis of at least three readings plus three standard deviations (the larger of the standard deviations for the wild type or mutated version should be used). In one embodiment, a substantial decrease in affinity is where the mean K_{D} for the mutated version measured on the basis of at least three readings is at least 3 fold greater than the mean K_{D} for wild type full length human TSLP measured on the basis of at least three readings. In another embodiment, a substantial decrease in affinity is where the mean K_{D} for the mutated version measured on the basis of at least three readings is at least 5 fold greater than the mean K_{D} for wild type full length human TSLP measured on the basis of at least three readings. In yet another embodiment, a substantial decrease in affinity is where the mean K_{D} for the mutated version measured on the basis of at least three readings is at least 10 fold greater than the mean K_{D} for wild type full length human TSLP measured on the basis of at least three readings. In one embodiment, altered affinity refers to reduced affinity.

In another embodiment, the invention provides a TSLP binding protein that binds to full length human TSLP (SEQ ID NO: 80) having one or more residues of Lys31, Ser32, Thr33, Phe35, Asn36, Asn37, Ser40, Cys41 and Ser42 mutated exhibiting altered affinity in comparison with full length human TSLP with no mutations. In another embodiment, the invention provides a TSLP binding protein that binds to full length human TSLP (SEQ ID NO: 80) having one or more residues of Lys31, Ser32, Thr33, Phe35 and Asn37 mutated exhibiting altered reduced affinity in comparison with full length human TSLP with no mutations. In yet another embodiment, the invention provides a TSLP binding protein that binds to full length human TSLP (SEQ ID NO: 80) having one or more residues of Lys31 and Phe35 mutated exhibiting altered affinity in comparison with full length human TSLP with no mutations.

In one embodiment, the invention provides a TSLP binding protein that binds to a peptide having the amino acid sequence set forth in SEQ ID NO: 37. In this context, the term “binds to” requires a binding response substantially greater than observed for any non-overlapping peptide of equivalent length derived from full length human TSLP. Binding may be assessed by any suitable method, for example ELISA. The peptides may be tagged (e.g. biotinylated) to facilitate the binding assay, but the sequence may not be extended by additional amino acids. In one embodiment, the peptide may have the sequence set forth as SEQ ID NO: 38. It should be noted that the requirement to bind to a peptide having the sequences specified does not necessarily mean that the binding protein may not interact with residues outside of this sequence or, for example, protect them from e.g. deuterium uptake provided that an “all or nothing” binding response is achieved (i.e., the levels of binding achieved by peptides having the sequence set forth in SEQ ID NO: 37 (or 38) being substantially greater than levels achieved by other, non-overlapping TSLP peptides). A substantially greater binding response in the context of this invention refers to the situation where the mean K_{D} for a peptide containing the sequence set forth in SEQ ID NO: 37 (or 38) on the basis of at least three readings is less than or equal to the mean K_{D} for the non-overlapping TSLP peptides measured on the basis of at least three readings minus one standard deviation (the largest standard deviation observed for any peptide being used). In one embodiment, the mean K_{D} for a peptide containing the sequence set forth in SEQ ID NO: 37 (or 38) on the basis of at least three readings is less than or equal to the mean K_{D} for the non-overlapping TSLP peptides measured on the basis of at least three readings minus two standard deviations (the largest standard deviation observed for any peptide being used). In a further embodiment, the mean K_{D} for a peptide containing the sequence set forth in SEQ ID NO: 37 (or 38) on the basis of at least three readings is less than or equal to the mean K_{D} for the non-overlapping TSLP peptides measured on the basis of at least three readings minus three standard deviations (the largest standard deviation observed for any peptide being used). In one embodiment, the mean K_{D} for a peptide containing the sequence set forth in SEQ ID NO: 37 (or 38) on the basis of at least three readings is at least 3 fold lower than the mean K_{D} for the non-overlapping TSLP peptides measured on the basis of at least three readings. In another embodiment, the mean K_{D} for a peptide containing the sequence set forth in SEQ ID NO: 37 (or 38) on the basis of at least three readings is at least 5 fold lower than the mean K_{D} for the non-overlapping TSLP peptides measured on the basis of at least three readings. In another embodiment, the mean K_{D} for a peptide containing the sequence set forth in SEQ ID NO: 37 (or 38) on the basis of at least three readings is at least 10 fold lower than the mean K_{D} for the non-overlapping TSLP peptides measured on the basis of at least three readings.

In one embodiment, the invention provides a TSLP binding protein that binds to full length human TSLP and results in peptides derived from full length human TSLP (SEQ ID NO: 80) containing part or the whole of the sequence from Tyr26 and Ser42 being more resistant to deuterium incorporation compared to corresponding peptides derived from uncomplexed full length human TSLP. In one embodiment, resistance to deuterium incorporation is assessed at a time point of between 30 and 300 seconds after dilution into deuterated buffer (e.g. at one of 30, 100, 120 or 300 seconds).

In certain of the above embodiments, said TSLP binding protein does not have the amino acid sequence set forth in SEQ ID NO: 9.

In further embodiments, said TSLP binding protein does not have the amino acid sequence set forth in any of SEQ ID NOs: 12, 23, 24, 25, 26, 27, 28, 29, 30, 31, 32, 33, 34, 35 or 36.

In certain embodiment, said TSLP binding protein is not a TSLP-binding protein comprising:
a. CDR1, CDR2 and CDR3 of SEQ ID NO: 9 or a variant of any one or all of these CDRs, wherein the CDR variant has 1, 2, or 3 amino acid modifications; or
b. an amino acid sequence at least 90% identical to the sequence of SEQ ID NO: 9;
   wherein said TSLP binding protein has an IC50 of less than or equal to 5 nM.

In one embodiment, said TSLP binding protein is not a TSLP binding protein that binds an epitope comprising the following residues of full length human TSLP (SEQ ID NO: 80): Tyr15, Lys31, Ser32, Thr33, Phe35, Asn36, Asn37, Ser40, Cys41, Ser42, Ser114, Gln115, Gln117, Gly118, Arg121, Arg122, Arg125, Pro126, Leu128 and Lys 129.

In one embodiment, said TSLP binding protein does not compete for binding to TSLP with a single variable domain of SEQ ID NO:9. In one embodiment, the TSLP used in the competition assay is full length human TSLP.

In one embodiment, the TSLP binding protein of the invention exhibits no significant binding to IL-7 (SEQ ID NO: 83). No significant binding to IL-7 refers to the situation where less than or equal to 10 response units is observed when the TSLP binding protein at a concentration of 160 nM in HBS-EP buffer is passed over human IL-7 (immobilised on a CM4 chip by primary amine coupling to a level of 1400 RU) at 25° C. in BIACORE™ T200. The method should be run in duplicate within the same BIAcore run and the curve double-referenced using a buffer injection curve.

In another embodiment, the TSLP binding protein of the invention exhibits cross reactivity with cynomolgus TSLP (SEQ ID NO:82). This is achieved where there is less than a 5 fold difference in IC50 between human full length TSLP (SEQ ID NO:80) and cynomolgus TSLP (SEQ ID NO:82) in inhibiting TSLP stimulated phosphorylation of Signal Transduction and Activator of Transcription 5 in the vaginal carcinoma cell line SW756 (ATCC). A suitable assay utilises SW756 cells seeded into 96-well plates at a density of 25,000 cells/well and incubated overnight at 37° C. in 5% CO₂ to allow adherence. Human (SEQ ID NO:80) or cynomolgus full length TSLP (SEQ ID NO:82), at an EC₇₅ concentration of 1 ng/ml, is pre-incubated with the TSLP binding protein at a concentration range of 0.05-1000 nM for one hour. The TSLP/binding protein complex is added to the cells and incubated for 30 minutes at 37° C. in 5% CO₂, followed by cell lysis. Lysates may be analysed by MesoScale Discovery (MSD) to quantify pSTAT5 as according to the manufacturer's protocol (K15163D-3) using an MSD Sector Imager 6000. Data may be plotted using a 4 parameter logistic fit model to obtain potency values.

In another embodiment, the TSLP binding protein of the invention exhibits no significant binding to the short isoform of human TSLP (SEQ ID NO: 81). Binding to the short isoform of human TSLP may be assessed using the ForteBio Octet label-free interaction analysis instrument at 25° C. Biotinylated short form TSLP diluted to 10 μg/ml in IgG-free PBS buffer is loaded onto streptavidin sensor tips to a level of 8-9 nm. Next, sensors are dipped into solutions of TSLP binding proteins at a concentration of 500 nM and the binding response is measured. A value of less than 0.1 nm in this assay when the TSLP binding protein is tested at 500 nM is indicative of no significant binding to the short isoform of human TSLP (SEQ ID NO: 81).

The TSLP binding protein may be an antibody or have a so-called alternative antibody format. Fragments of antibodies or fragments of proteins having an alternative antibody format, which fragments retain the specified binding properties are also included.

The term “antibody” is used herein in the broadest sense to refer to proteins with an immunoglobulin scaffold and includes monoclonal, recombinant, synthetic, polyclonal, chimeric, human, humanised, multispecific antibodies, including bispecific antibodies, and heteroconjugate antibodies and modified versions of any of the foregoing. Antibodies may have an IgG, IgM, IgA, IgD or IgE scaffold, for example an IgG, or IgA scaffold. In one embodiment, an antibody may comprise an IgG scaffold selected from IgG1, IgG2, IgG3, IgG4 or IgG4PE. For example, the scaffold may be IgG1.

Proteins having an alternative antibody format have a non-immunoglobulin scaffold having loops connecting elements of secondary structure which can be engineered to include CDR regions, for example CDR regions from antibodies identified as having appropriate binding properties when incorporated into an immunoglobulin scaffold. Non immunoglobulin scaffolds include CTLA-4 (Evibodies; Journal Immunological Methods 248(1-2): 31-45, 2001), lipocalin, Protein A derived molecules such as Z-domain of Protein A (Affibodies, Protein Eng Des Sel 17: 455-462, 2004 and EP1641818), A-domain (Avimer/Maxibody), heat shock proteins such as GroEI and GroES, transferrin (trans-body), ankyrin repeat protein (DARPin), peptide aptamer, C-type lectin domain (Tetranectin), human γ-crystallin and human ubiquitin (affilins), PDZ domains, scorpion toxin kunitz-type domains of human protease inhibitors, and fibronectin/adnectin. These scaffolds are subjected to protein engineering to arrange the CDRs in a functional manner (for a summary of alternative antibody formats see Holliger and Hudson, Nature Biotechnology, 2005, Vol 23, No 9, 1126-1136).

Antibody fragments may comprise one or more domains of a full length antibody. For example, antibody fragments include Fab, F(ab′)2, Fv, disulphide linked Fv, single chain Fv, disulphide-linked scFv, diabodies, TANDAB′, and single variable domains that retain the specified binding properties in the absence of other domains are included (single variable domains that are capable of binding antigen in the absence of other domains are sometimes referred to as domain antibodies). Single variable domains include VH, VHH, VL and modified antibody variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain),

A single variable domain may be a human single variable domain, but also includes single variable domains from other species such as rodent, nurse shark and Camelid VHH domains. Single variable domains from other species may be humanised according to standard techniques available in the art.

Numerous custom antibody production services (e.g. Genscript) exist. Custom antibody production services could be used to raise antibodies against peptides having the sequence set out in SEQ ID NO: 37 or SEQ ID NO: 38. The resulting antibodies could then be screened for the required binding without undue burden. Antibody fragments, proteins having an alternative antibody format or fragments of proteins having an alternative antibody format may readily be derived from antibodies identified in this way. Alternatively, TSLP binding proteins could be identified by screening for proteins having the required binding properties from libraries according to conventional methods.

TSLP binding proteins may be prepared by any of a number of conventional techniques. For example, TSLP binding proteins may be purified from cells that naturally express them (e.g., an antibody can be purified from a hybridoma that produces it), or produced in recombinant expression systems.

A number of different expression systems and purification regimes can be used to generate the antigen binding protein of the disclosure. Generally, host cells are transformed with a recombinant expression vector encoding the desired antigen binding protein. A wide range of host cells can be employed, including Prokaryotes (including Gram negative or Gram positive bacteria, for example Escherichia coli, Bacilli sp., Pseudomonas sp., Corynebacterium sp.), Eukaryotes including yeast (for example Saccharomyces cerevisiae, Pichia pastoris), fungi (for example Aspergillus sp.), or higher Eukaryotes including insect cells and cell lines of mammalian origin (for example, CHO, Perc6, HEK293, HeLa).

The host cell may be an isolated host cell. The host cell is usually not part of a multicellular organism (e.g., plant or animal). The host cell may be a non-human host cell.

Appropriate cloning and expression vectors for use with bacterial, fungal, yeast, and mammalian cellular hosts and methods of cloning are known in the art.

The cells can be cultured under conditions that promote expression of the antigen binding protein, and the polypeptide recovered by conventional protein purification procedures. The antigen binding proteins contemplated for use herein include substantially homogeneous antigen binding proteins substantially free of contaminating materials.

The skilled person will appreciate that, upon production of the TSLP binding protein, in particular depending on the cell line used and particular amino acid sequence of the antigen binding protein, post-translational modifications may occur. Such post-translational modifications may include the cleavage of certain leader sequences, the addition of various sugar moieties in various glycosylation patterns, deamidation (for example at an asparagine or glutamine residue), oxidation (for example at a methionine, tryptophan or free cysteine residue), disulfide bond scrambling, isomerisation (for example at an aspartic acid residue), C-terminal lysine clipping (for example from one or both heavy chains), and N-terminal glutamine cyclisation (for example, in the heavy and/or light chain). The present disclosure encompasses the use of antibodies which have been subjected to, or have undergone, one or more post-translational modifications. The modification may occur in a CDR, the variable framework region, or the constant region. The modification may result in a change in charge of the molecule.

TSLP expression and/or function is linked to a number of inflammatory disorders, predominantly those allergic in nature (characterised by immunoglobulin E (IgE)-related immunological responses), but also non-allergic diseases. These diseases include, but are not limited to, asthma (including severe asthma), idiopathic pulmonary fibrosis, atopic dermatitis (AD), allergic conjunctivitis, allergic rhinitis (AR), Netherton syndrome (NS), eosinophilic esophagitis (EoE), food allergy, allergic diarrhoea, eosinophilic gastroenteritis, allergic bronchopulmonary aspergillosis (ABPA), allergic fungal sinusitis, cancer (e.g., breast, pancreas, B-cell acute lymphoblastic leukaemia), rheumatoid arthritis, COPD, systemic sclerosis, keloids, ulcerative colitis, chronic rhinosinusitis (CRS), and nasal polyposis. In addition as TSLP stimulates the production of the type 2 cytokines IL-5, IL-13 and IL-4, it is also implicated in diseases to which these cytokines have been linked, such as, but not limited to, asthma, allergic rhinitis, chronic eosinophilic pneumonia, eosinophilic bronchitis, allergic bronchopulmonary aspergillosis, coeliac disease, eosinophilic gastroenteritis, Churg-Strauss syndrome, eosinophilic myalgia syndrome, hypereosinophilic syndrome, eosinophilic granulomatosis with polyangiitis, eosinophilic esophagitis, and inflammatory bowel disease.

In one aspect, the invention provides a TSLP binding protein as described herein for use in treating a disease associated with TSLP signalling. In one embodiment, the subject is a human. Use of a TSLP binding protein as defined herein in the manufacture of a medicament for the treatment of a disease associated with TSLP signalling is also provided. In one embodiment, the subject is a human. The invention provides a method of treating a disease associated with TSLP signalling in a human patient in need thereof, the method comprising administering a TSLP binding protein as defined herein to the human patient.

In one embodiment, the disease associated with TSLP signalling is selected from the group consisting of: asthma, idiopathic pulmonary fibrosis, atopic dermatitis, allergic conjunctivitis, allergic rhinitis, Netherton syndrome, eosinophilic esophagitis (EoE), food allergy, allergic diarrhoea, eosinophilic gastroenteritis, allergic bronchopulmonary aspergillosis (ABPA), allergic fungal sinusitis, cancer, rheumatoid arthritis, COPD, systemic sclerosis, keloids, ulcerative colitis, chronic rhinosinusitis (CRS), nasal polyposis, chronic eosinophilic pneumonia, eosinophilic bronchitis, coeliac disease, Churg-Strauss syndrome, eosinophilic myalgia syndrome, hypereosinophilic syndrome, eosinophilic granulomatosis with polyangiitis and inflammatory bowel disease. In another embodiment, the disease associated with TSLP signalling is selected from the group consisting of: asthma, idiopathic pulmonary fibrosis, atopic dermatitis, allergic conjunctivitis, allergic rhinitis, Netherton syndrome, eosinophilic esophagitis (EoE), food allergy, allergic diarrhoea, eosinophilic gastroenteritis, allergic bronchopulmonary aspergillosis (ABPA), allergic fungal sinusitis, cancer, rheumatoid arthritis, COPD, systemic sclerosis, keloids, ulcerative colitis, chronic rhinosinusitis (CRS) and nasal polyposis. In yet another embodiment, the disease associated with TSLP signalling is asthma. In an additional embodiment, the disease associated with TSLP signalling is severe asthma or mild to moderate asthma.

Severe asthma is asthma that can only be controlled using the maximally recommended doses of the most effective combinations of controller medication(s) and bronchodilator(s) recommended by treatment guidelines, or which remains uncontrolled despite this therapy. It will be appreciated that the most effective combinations may change over time, but can be readily ascertained by reference to published treatment guidelines (e.g. GINA). Similarly, maximally recommended doses may be identified by reference to these treatment guidelines. In one embodiment, the controller medication may be selected from the group consisting of: inhaled corticosteroid and systemic corticosteroid. In one embodiment, a bronchodilator may be selected from the group consisting of: a long acting beta agonist, a leukotriene modifier and theophylline. In one embodiment, severe asthma is asthma which requires treatment with high dose inhaled corticosteroid and either long acting beta agonist or leukotriene modifier/theophylline, or treatment with systemic corticosteroid to prevent it from becoming “uncontrolled” or which remains “uncontrolled” despite this therapy.

Mild to moderate asthma is asthma that can be controlled using first line treatment options recommended by treatment guidelines (at recommended doses). In one embodiment, the first line treatment is regular low dose inhaled corticosteroid/leukotriene receptor antagonist/theophylline plus as needed short acting beta agonist, or medium dose inhaled corticosteroid/long acting beta agonist as maintenance treatment plus as needed short acting beta agonist.

In these embodiments, treatment can be therapeutic, prophylactic or preventative. Treatment encompasses alleviation, reduction, or prevention of at least one aspect or symptom of a disease and encompasses prevention or cure of the diseases described herein.

The TSLP binding protein described herein is used in an effective amount for therapeutic, prophylactic or preventative treatment. A therapeutically effective amount of the TSLP binding protein described herein is an amount effective to ameliorate or reduce one or more symptoms of, or to prevent or cure, a disease.

A TSLP binding protein of the invention may be administered alone or in combination with other therapeutic agents. The TSLP binding protein and one or more other therapeutic agents may be administered separately, simultaneously or sequentially. When used in combination, the dose of TSLP binding protein may differ from the dose used for monotherapy.

A TSLP binding protein of the invention may be administered in combination with inhaled, intranasal or parenteral corticosteroids such as fluticasone furoate, fluticasone propionate, budesonide, ciclesonide, beclomethasone dipropionate, mometasone furoate, triamcinolone acetonide and prednisolone. In one embodiment, a TSLP binding protein of the invention may be administered as a fixed dose combination with an inhaled corticosteroid such as a fixed dose combination with fluticasone furoate or fluticasone propionate.

A TSLP binding protein of the present invention may be administered in combination with a bronchodilator such as a beta-2 adrenoreceptor agonist and/or a muscarinic antagonist. Suitable beta-2 adrenoreceptor agonists include vilanterol, salmeterol, salbutamol, formoterol, salmefamol, fenoterol, carmoterol, etanterol, naminterol, clenbuterol, pirbuterol, flerbuterol, reproterol, bambuterol, indacaterol, terbutaline, and salts thereof. Suitable muscarinic antagonists include umeclidinium, tiotropium, glycopyrrolate, ipratropium, and salts thereof such as the bromide salt of umeclidinium. In one embodiment, a TSLP binding protein of the invention may be administered as a fixed dose combination with a beta-2 adrenoreceptor agonist and/or a muscarinic antagonist such as a fixed dose combination with vilanterol trifenatate, umeclidinium bromide, or the dual combination of vilanterol trifenatate and umeclidinium bromide.

A TSLP binding protein of the present invention may be administered with a combination of one or more bronchodilators and an inhaled steroid. Such combinations may include dual combinations such as fluticasone furoate and vilanterol trifenatate, fluticasone furoate and umeclidinium bromide, fluticasone propionate and salmeterol, budesonide and formoterol, mometasone and formoterol, and triple therapy such as fluticasone furoate, vilanterol trifenatate and umeclidinium bromide. In one embodiment, a TSLP binding protein of the present invention may be administered as a fixed dose combination with an inhaled corticosteroid and one or more bronchodilators, such as a fixed dose combination with fluticasone furoate and vilanterol trifenatate, or fluticasone propionate and salmeterol, or fluticasone furoate and umeclidinium bromide, or fluticasone furoate, vilanterol trifenatate and umeclidinium bromide.

A TSLP binding protein of the present invention may be administered in combination with anti-leukotriene antagonists such as montelukast, zafirlukast and pranlukast; PDE4 inhibitors such as roflumilast; xanthenes; anti-IgE antibodies such as omalizumab; antagonists of IL-5 such as mepolizumab, benralizumab and reslizumab; antagonists of IL-13 such as lebrikizumab and tralokinumab; antagonists of IL-4/IL-13 such as dupilumab; antagonists of IL-6 such as sirukumab and antagonists of IL-1, IL-4, IL-33, IL-25 and TNF-α.

A TSLP binding protein of the present invention may be administered in combination with an anti-histamine such as cetirizine hydrochloride, levocetirizine, desloratidine, loratidine, fexofenadine hydrochloride or azelastine.

A TSLP binding protein of the present invention may be administered in combination with pirfenidone or nintedanib or an avB6 antagonist, for example, those disclosed in WO2014/154725.

The TSLP binding protein as described herein may be incorporated into pharmaceutical compositions for use in the treatment of the human diseases described herein. Such compositions optionally comprise a pharmaceutically acceptable carrier as known and called for by acceptable pharmaceutical practice.

Pharmaceutical compositions may be administered by injection or continuous infusion (examples include, but are not limited to, intravenous, intraperitoneal, intradermal, subcutaneous, intramuscular and intraportal). Pharmaceutical compositions may be suitable for topical administration (which includes, but is not limited to, epicutaneous, inhaled, intranasal or ocular administration) or enteral administration (which includes, but is not limited to, oral or rectal administration). In one embodiment, the pharmaceutical composition is inhaled. Pharmaceutical compositions may comprise between 1 μg and 15 mg, for example between 1 μg and 10 mg. In an embodiment the pharmaceutical composition comprises between 500 μg and 2.5 mg, for example between 250 μg and 5 mg of TSLP binding protein.

Methods for the preparation of such pharmaceutical compositions are well known to those skilled in the art. Other excipients may be added to the composition as appropriate for the mode of administration and the particular protein used. Examples of different excipients and their uses are described in Lowe et al., Adv Protein Chem Struct Biol, 84, 41-61 (2011).

Effective doses and treatment regimes for administering the TSLP binding protein may be dependent on factors such as the age, weight and health status of the patient, disease to be treated and other concomitant medications. Such factors are within the purview of the attending physician. Guidance in selecting appropriate doses may be found in e.g Bai et al., Clin Pharmacokinet, 51, 119-35 (2012).

### Non-Epitope Related Aspects

The disclosure relates to a TSLP binding protein, in particular, an anti-TSLP single variable domain (anti-TSLP domain antibody or dAb). The key features/characteristics that make the TSLP binding protein of the disclosure an ideal candidate for the treatment or prevention of asthma and other diseases described herein are described below:

Size:

An anti-TSLP single variable domain is smaller than a monoclonal antibody, f(Ab′)₂ or fAb fragment, and, therefore, has the advantage as a therapeutic for inhaled delivery because a greater number of antagonist dAb molecules can be delivered to the lung (per mg of protein) compared with a larger molecule. Unmodified domain antibodies (that lack an Fc domain) are rapidly cleared from the systemic compartment by renal filtration, and, therefore, may be suitable for inhaled delivery to maximise lung exposure while minimising systemic exposure.

Affinity/Potency:

One embodiment of the present invention provides a suitably high affinity/potency antagonist to enable neutralisation of human TSLP. If the affinity/potency is not sufficiently high, then the binding protein may not effectively neutralise TSLP, and/or the dose required to inhibit TSLP in the body is high and may be not achievable or commercially viable. The dAb molecules described herein were selected and optimised to have an affinity/potency which provides neutralisation of human TSLP in the assays tested, and these dAbs are suitable for neutralisation of TSLP in humans, for example in the lung after inhaled delivery.

Mechanism:

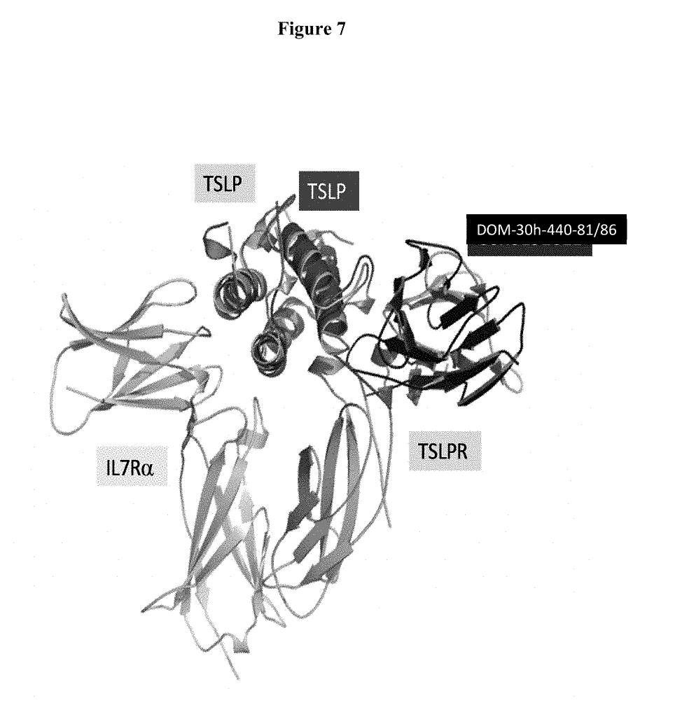

Because the TSLP receptor complex is present on antigen-presenting cells, one aspect of the present invention provides that a TSLP binding protein prevents TSLP from binding to the TSLP receptor complex. TSLP antagonists that act by preventing recruitment of the IL-7Rα chain, or by binding directly to TSLPR (or IL-7Rα), may be internalised and processed as antigens more effectively than a TSLP antagonist that binds TSLP and stays in solution as a complex with TSLP. Example 14 and FIG. 7 show that DOM30h-440-81/86 prevents TSLP from binding to the TSLP receptor complex and identifies the residues that form the epitope for DOM30h-440-81/86 on human full length TSLP. Residues forming the paratope for this epitope are also identified.

Selectivity:

The TSLP binding protein described herein include those that bind and neutralise human TSLP, but do not have significant binding to the related cytokine IL-7. One aspect of this invention provides such TSLP binding proteins, because IL-7 has discrete actions in the body, including as a growth factor for lymphoid cells. Antagonists that neutralise the activity of both TSLP and IL-7 are likely to result in different effects in humans compared with a TSLP-selective antagonist.

Inhibition of Human TSLP:

The TSLP binding proteins described herein bind to and neutralise full length human TSLP produced recombinantly (e.g. expressed from E. coli or HEK—human embryonic kidney—cells) and native human TSLP (e.g. produced by stimulating human lung fibroblasts with inflammatory cytokines). For example, DOM30h-440-81/86 binds to and neutralises both full length recombinant human TSLP (expressed from E. coli (Table 9) or HEK cells (Table 10)) and native human TSLP expressed from human cells (Example 7). In one embodiment of the present invention, the native human TSLP may be glycosylated, whereas the recombinant human TSLP expressed in E. coli is not glycosylated. Example 9 shows that DOM30h-440-81/86 binds to full length human TSLP and not to the short isoform.

Expression Level:

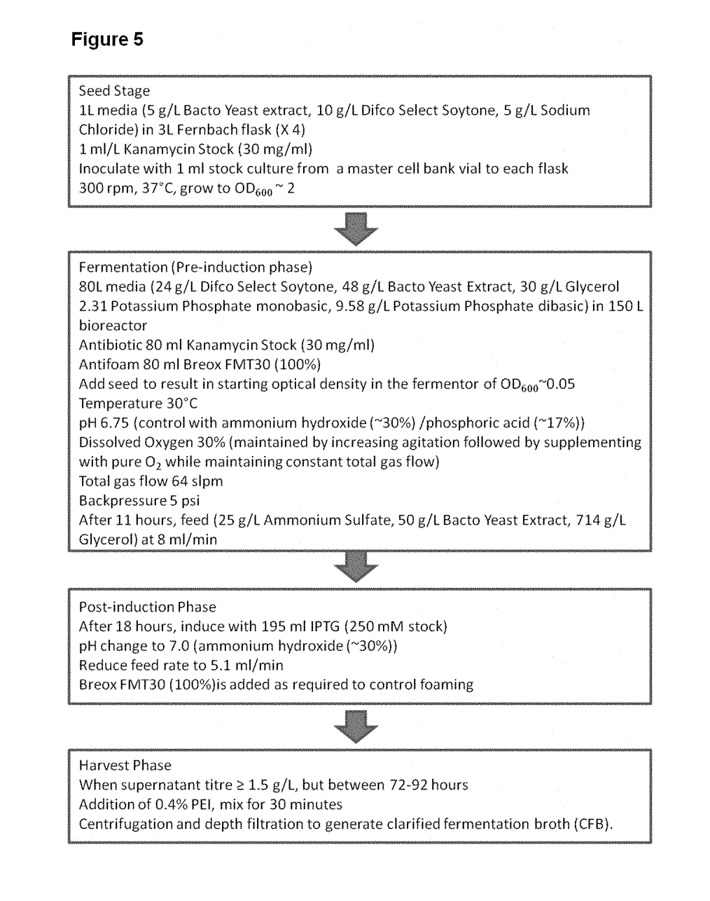

To facilitate the manufacture of large amounts of an anti-TSLP single variable domain antagonist, the dAb molecules can be expressed efficiently. The dAb molecules described herein can be expressed from E. coli at a high level. Example 10 and FIG. 5 describe the fermentation process for production of DOM30h-440-81/86.

Downstream Purification Process:

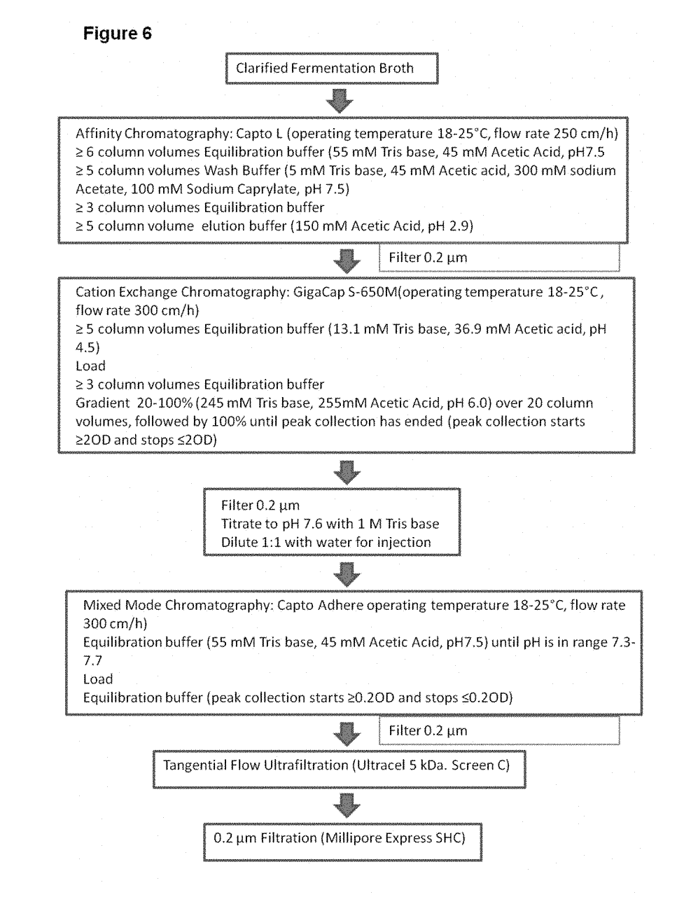

To facilitate the manufacture of large amounts of a TSLP antagonist, one aspect of the present invention provides that the dAb anti-TSLP single variable domains can be purified efficiently from cell supernatants. The dAb molecules described herein can be purified efficiently using affinity chromatography methods because of their high affinity for protein L. Example 10 and FIG. 6 describe the downstream purification process for production of DOM30h-440-81/86.

Biophysical Characteristics:

To enable ease of administration to humans, one embodiment of the present invention provides that a TSLP binding protein has both a high solubility and low levels of aggregation and/or fragmentation in solution. anti-TSLP dAb molecules exemplified by DOM30h-440-81/86 showed high thermal stability, resistance to pH and temperature stress and low levels of aggregation.

Stability to Spray Drying:

To facilitate the delivery of a dry powder formulation of a TSLP binding protein, one aspect of the present invention provides that the anti-TSLP single variable domain is stable to the freeze-drying and/or spray-drying processes. Stability to temperature stress and shear stress may be maximised by selecting a TSLP that has a high melting temperature (Tm). In particular, DOM30h-440-81/86 has among the highest Tm of those tested.

The embodiments of the present invention describe a way which enables a clear and concise specification to be written. It is intended and should be appreciated that embodiments may be variously combined or separated without parting from the disclosure.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook, et al., Molecular Cloning: A Laboratory Manual, 2d ed. (1989) Cold Spring Harbor Laboratory Press, Cold Spring Harbor, N.Y. and Ausubel, et al., Short Protocols in Molecular Biology (1999) 4th Ed, John Wiley & Sons, Inc., which are incorporated herein by reference) and chemical methods.

The term “TSLP binding protein”, as used herein, refers to antibodies and other protein constructs, such as domains, which are capable of binding to TSLP. The terms “antigen binding protein” and “TSLP binding protein” are used interchangeably in this specification.

“TSLP”, as used herein, refers to naturally occurring or endogenous mammalian TSLP proteins and to proteins having an amino acid sequence which is the same as that of a naturally occurring or endogenous TSLP Protein (e.g., recombinant proteins, synthetic proteins). Accordingly, as defined herein, this term includes mature TSLP protein, polymorphic or allelic variants and other isoforms of TSLP and modified and unmodified forms of the foregoing (e.g., lapidated, glycosylated). Human TSLP is described, for example, in Liu et al (Annu. Rev. Immunol. 2007. 25:193-219). Mature human TSLP (also known as full length TSLP) is a 131-amino acid (15 KD) four α-helix bundle cytokine. Two other splice variants of human TSLP are predicted: one splice variant encodes the same 131-amino acid secreted protein, and the other splice variant gives rise to a truncated C-terminal isoform of approximately 60 amino acids.

The term, “anti-TSLP”, with reference to a single variable domain or polypeptide means a moiety that recognises and binds TSLP. In one embodiment, an “anti-TSLP” specifically recognises and/or specifically binds to human TSLP. In another embodiment, the TSLP binding protein binds to residues of human TSLP that are involved in binding of TSLP to the TSLPR:IL-7Rα complex.

The term “antibody”, is used herein in the broadest sense, refers to molecules with an immunoglobulin-like domain (for example, IgG, IgM, IgA, IgD or IgE) and includes monoclonal, recombinant, polyclonal, chimeric, human, humanised, multispecific antibodies, including bispecific antibodies, and heteroconjugate antibodies; a single variable domain (e.g., VH, VHH, VL), antigen binding antibody fragments, Fab, F(ab′)₂, Fv, disulphide linked Fv, single chain Fv, disulphide-linked scFv, diabodies, TANDABS™, etc., and modified versions of any of the foregoing.

Alternative antibody formats include alternative scaffolds in which the one or more CDRs of the antigen binding protein can be arranged onto a suitable non-immunoglobulin protein scaffold or skeleton, such as an affibody, a SpA scaffold, an LDL receptor class A domain, an avimer or an EGF domain.

The term “domain” refers to a folded protein structure which retains its tertiary structure independent of the rest of the protein. Generally, domains are responsible for discrete functional properties of proteins, and in many cases, may be added, removed or transferred to other proteins without loss of function of the remainder of the protein and/or of the domain.

The term “single variable domain” refers to a folded polypeptide domain comprising sequences characteristic of antibody variable domains. It therefore includes complete antibody variable domains such as VH, VHH and VL and modified antibody variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A single variable domain is capable of binding an antigen, in this case TSLP, or epitope independently of a different variable region or domain. A “domain antibody” or “dAb” may be considered the same as a “single variable domain”. A single variable domain may be a human single variable domain, but also includes single variable domains from other species such as rodent, nurse shark and Camelid VHH dAbs. Camelid VHH are immunoglobulin single variable domain polypeptides that are derived from species including camel, llama, alpaca, dromedary, and guanaco, which produce heavy chain antibodies naturally devoid of light chains. Such VHH domains may be humanised according to standard techniques available in the art, and such domains are considered to be “single variable domains”. As used herein VH includes camelid VHH domains. In a particular embodiment the single variable domain is a VL domain. In a particular embodiment the VL domain is a V_{K} domain. In one embodiment, the single variable domain is non-naturally occurring.

An antigen binding fragment may be provided by means of arrangement of one or more CDRs on non-antibody protein scaffolds. “Protein Scaffold”, as used herein, includes, but is not limited to, an immunoglobulin (Ig) scaffold, for example, an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions.

The protein scaffold may be an Ig scaffold, for example, an IgG, or IgA scaffold. The IgG scaffold may comprise some or all the domains of an antibody (i.e. CH1, CH2, CH3, VH, VL). The antigen binding protein may comprise an IgG scaffold selected from IgG1, IgG2, IgG3, IgG4 or IgG4PE. For example, the scaffold may be IgG1. The scaffold may consist of, or comprise, the Fc region of an antibody, or is a part thereof.

The protein scaffold may be a derivative of a scaffold selected from the group consisting of CTLA-4, lipocalin, Protein A derived molecules such as Z-domain of Protein A (Affibody, SpA), A-domain (Avimer/Maxibody); heat shock proteins such as GroEl and GroES; transferrin (trans-body); ankyrin repeat protein (DARPin); peptide aptamer; C-type lectin domain (Tetranectin); human γ-crystallin and human ubiquitin (affilins); PDZ domains; scorpion toxin kunitz type domains of human protease inhibitors; and fibronectin/adnectin; which has been subjected to protein engineering in order to obtain binding to an antigen, such as TSLP, other than the natural ligand.

A “dAb conjugate” refers to a composition comprising an anti-TSLP dAb, as disclosed herein, to which a drug is chemically conjugated by means of a covalent or noncovalent linkage. In one embodiment, the dAb and the drug are covalently bonded. Such covalent linkage could be through a peptide bond or other means such as via a modified side chain. The noncovalent bonding may be direct (e.g., electrostatic interaction, hydrophobic interaction) or indirect (e.g., through noncovalent binding of complementary binding partners (e.g., biotin and avidin)), wherein one partner is covalently bonded to drug and the complementary binding partner is covalently bonded to the dAb. When complementary binding partners are employed, one of the binding partners can be covalently bonded to the drug directly or through a suitable linker moiety, and the complementary binding partner can be covalently bonded to the dAb directly or through a suitable linker moiety.

As used herein, “dAb fusion” refers to a fusion protein that comprises an anti-TSLP dAb, as disclosed herein, and a polypeptide drug (which could be a dAb or mAb). The dAb and the polypeptide drug are present as discrete parts (moieties) of a single continuous polypeptide chain.

In one embodiment, antigen binding proteins of the present disclosure show cross-reactivity between human TSLP and TSLP from another species, such as cynomolgus monkey TSLP. In another embodiment, the antigen binding proteins of the disclosure specifically bind human and monkey TSLP. Such cross-reactivity is useful, as drug development typically requires testing of lead drug candidates in animal systems before the drug is tested in humans. The provision of a drug that can bind human and monkey species allows one to test results in these systems and make side-by-side comparisons of data using the same drug. Providing such a drug avoids the complication of needing to find a drug that works against rodent or monkey TSLP and a separate drug that works against human TSLP, and also avoids the need to compare results in humans and animals using non-identical drugs. In an embodiment, the antigen binding proteins of the disclosure specifically bind human and cynomolgus monkey TSLP.

Optionally, the binding affinity of the antigen binding protein for at least cynomolgus TSLP and the binding affinity for human TSLP, differ by no more than a factor of 2, 5, 10, 50 or 100. In one embodiment, the binding affinity for cynomologus TSLP and the binding affinity for human TSLP differ by no more than a factor of 5. In another embodiment, the binding affinity for cynomologus TSLP and the binding affinity for human TSLP differ by no more than a factor of 2.

Affinity is the strength of binding of one molecule, e.g., an antigen binding protein of the disclosure, to another, e.g., its target antigen, at a single binding site. The binding affinity of an antigen binding protein to its target may be determined by equilibrium methods (e.g., enzyme-linked immunoabsorbent assay (ELISA) or radioimmunoassay (RIA)), or kinetics (e.g., Biacore™ analysis). For example, the Biacore™ methods described in Example 1 may be used to measure binding affinity.

In one embodiment, the equilibrium dissociation constant (KD) of the antigen binding protein-TSLP interaction, in particular, the antigen binding protein-human TSLP interaction, is 100 nM or less, 10 nM or less, 5 nM or less, 2 nM or less or 1 nM or less. In another embodiment, the KD (for human TSLP) is less than 2 nM. Alternatively, the KD (for human TSLP) may be between 0.5 and 2 nM. The KD (for human TSLP) may be between 500 pM and 1 nM. A skilled person will appreciate that the smaller the KD numerical value, the stronger the binding. The reciprocal of KD (i.e., 1/KD) is the equilibrium association constant (KA) having units M⁻¹. A skilled person will appreciate that the larger the KA numerical value, the stronger the binding.

The dissociation rate constant (kd) or “off-rate” describes the stability of the antigen binding protein-TSLP complex, i.e., the fraction of complexes that decay per second. For example, a kd of 0.01 s⁻¹ equates to 1% of the complexes decaying per second. In an embodiment, the dissociation rate constant (kd) is 1×10's⁻¹ or less. The kd may be between 1×10⁻⁴ s⁻¹ and 1×10⁻³ s⁻¹.

The association rate constant (ka) or “on-rate” describes the rate of antigen binding protein-TSLP complex formation. In an embodiment, the association rate constant (for human TSLP) (ka) is greater than 1×10⁵ M⁻¹ s⁻¹. In an embodiment, the ka for human TSLP is between 4×10⁵ M⁻¹ s⁻¹ and 8×10⁵ M⁻¹ s⁻¹.

Competition between the TSLP binding protein and a reference single variable domain, e.g., SEQ ID NO:9, may be determined by competition ELISA, FMAT or BIAcore. In one embodiment, the competition assay is carried out by surface plasmon resonance (SPR) There are several possible reasons for this competition: the two proteins may bind to the same or overlapping epitopes, there may be steric inhibition of binding, or binding of the first protein may induce a conformational change in the antigen that prevents or reduces binding of the second protein. In one embodiment, the TSLP binding protein of the disclosure competes for binding to TSLP with a single variable domain of SEQ ID NO:9. In one embodiment, the TSLP used in the competition assay is full length human TSLP.

The reduction or inhibition in biological activity may be partial or total. A neutralising antigen binding protein may neutralise the activity of TSLP by at least 50%, 55%, 60%, 65%, 70%, 75%, 80%, 82%, 84%, 86%, 88%, 90%, 92%, 94%, 95%, 96%, 97%, 98%, 99% or 100% relative to TSLP activity in the absence of the TSLP binding protein.

The term “neutralises” as used herein, means that the biological activity of TSLP is reduced in the presence of an antigen binding protein as described herein in comparison to the activity of TSLP in the absence of the antigen binding protein, in vitro or in vivo. Neutralisation may be due to one or more of blocking TSLP binding to its receptor, preventing TSLP from activating its receptor, down regulating TSLP or its receptor, or affecting effector functionality. For example, the receptor binding assay (RBA) method described in Example 1 may be used to assess the neutralising capability of a TSLP binding protein.

The term, “no significant binding to IL-7”, as used herein, means that no binding (of the TSLP binding protein) is detectable to IL-7 using the binding assay referred to in the specification, and in particular, in the SPR assay referred to in Example 1.

“CDRs” are defined as the complementarity determining region amino acid sequences of an antigen binding protein. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs. In the case of a single variable domain there are three CDRs.

Throughout this specification, amino acid residues in variable domain sequences and full length antibody sequences are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 5th Ed., U.S. Department of Health and Human Services, National Institutes of Health (1991).

As discussed above, there are alternative numbering conventions for amino acid residues in variable domain sequences and full length antibody sequences. There are also alternative numbering conventions for CDR sequences, for example those set out in Chothia et al. (1989) Nature 342: 877-883. The structure and protein folding of the antibody may mean that other residues are considered part of the CDR sequence and would be understood to be so by a skilled person.

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. Table 3 below represents one definition using each numbering convention for each CDR or binding unit. The Kabat numbering scheme is used in Table 1 to number the variable domain amino acid sequence. It should be noted that some of the CDR definitions may vary depending on the individual publication used.

TABLE 3

| | | | | | Mini- |
|---|---|---|---|---|---|
| | | | | | mum |
| | | Chothia | | Contact | binding |
| | Kabat CDR | CDR | AbM CDR | CDR | unit |
| H1 | 31-35/ | 26-32/ | 26-35/ | 30-35/ | 31-32 |
| | 35A/35B | 33/34 | 35A/35B | 35A/35B | |
| H2 | 50-65 | 52-56 | 50-58 | 47-58 | 52-56 |
| H3 | 95-102 | 95-102 | 95-102 | 93-101 | 95-101 |
| L1 | 24-34 | 24-34 | 24-34 | 30-36 | 30-34 |
| L2 | 50-56 | 50-56 | 50-56 | 46-55 | 50-55 |
| L3 | 89-97 | 89-97 | 89-97 | 89-96 | 89-96 |

Accordingly, a TSLP binding protein is provided, which comprises any one or a combination of the CDRs from SEQ ID NO:9, in particular as set out in Table 2 above, or a CDR variant thereof. In an embodiment CDRL1 consists of any one of SEQ ID NO: 1, 2 or 3. In an embodiment CDRL2 consists of any one of SEQ ID NO: 4, 5 or 6. In one embodiment, CDRL3 consists of any one of SEQ ID NO: 7 or 8.

CDRs or minimum binding units may be modified by at least one amino acid substitution, deletion or addition to form CDR variants, wherein the variant antigen binding protein substantially retains the biological characteristics of the unmodified protein, such as the ability to specifically bind human and cynomologus monkey TSLP.

It will be appreciated by one of skill in the art that each of the CDRs may be modified alone or in combination with any other CDR, in any permutation or combination.

A CDR variant includes an amino acid sequence modified by at least one amino acid, wherein said modification can be chemical or a partial alteration of the amino acid sequence (for example by no more than 5 amino acids), which modification permits the variant to retain the biological characteristics of the unmodified sequence. A partial alteration of the CDR amino acid sequence may be by deletion or substitution of one to several amino acids, or by addition or insertion of one to several amino acids, or by a combination thereof (for example by no more than 5 amino acids). The CDR variant may contain 1, 2, 3, 4, or 5 amino acid substitutions, additions or deletions, in any combination, in the amino acid sequence.

Typically, the modification is a substitution or a conservative substitution, for example, as shown in Table 1 above. In one embodiment, a CDR is modified by the substitution of up to 5 amino acids, for example up to 4 amino acids, for example up to 3 amino acids, for example, 1 or 2 amino acids, for example 1 amino acid. In an embodiment each of the three CDRs of a single variable domain is modified, independently of the other two CDRs, by 2, 1 or none amino acid residues. In an embodiment only CDR1 and/or CDR2 are modified.

In a particular example, in a variant CDR, the amino acid residues of the minimum binding unit may remain the same, but the flanking residues that comprise the CDR as part of the Kabat or Chothia definition(s) may be substituted with a conservative amino acid residue. In one embodiment, an anti-TSLP single variable domain comprises up to three variant CDRs from SEQ ID NO:9, wherein CDR1 comprises the minimum binding unit of SEQ ID NO:3, CDR2 comprises the minimum binding unit of SEQ ID NO:6, and CDR3 comprises the minimum binding unit of SEQ ID NO:8.

Such antigen binding proteins comprising modified CDRs or minimum binding units as described above may also be referred to herein as “functional CDR variants” or “functional binding unit variants”.

In one embodiment, a TSLP binding protein comprises CDR1, CDR2 and CDR3 or a variant of CDR1, CDR2 and/or CDR3 of SEQ ID NO:9 and either (i) competes with SEQ ID NO:9 for binding to TSLP and/or (ii) has a KD for TSLP of 2 nM or less.

The term “epitope”, as used herein, refers to that portion of the antigen that makes contact with a particular binding domain of the antigen binding protein. An epitope may be linear or conformational/discontinuous. A conformational or discontinuous epitope comprises amino acid residues that are separated by other sequences, i.e., not in a continuous sequence in the antigen's primary sequence. Although the residues may be from different regions of the peptide chain, they are in close proximity in the three-dimensional structure of the antigen. In the case of multimeric antigens, a conformational or discontinuous epitope may include residues from different peptide chains. Particular residues comprised within an epitope can be determined through computer modelling programs or via three-dimensional structures obtained through methods known in the art, such as X-ray crystallography. In one embodiment, residues comprising the epitope of a TSLP binding protein are those residues on TSLP that become more inaccessible to solvent upon binding to said TSLP binding protein. In one embodiment, epitope residues for a particular TSLP binding protein may be identified using the Qt-PISA v2.0.1 software (Protein Interfaces, Complexes and Assemblies; Krissinel and Henrick (2007) as being those residues on full length human TSLP where greater than or equal to 20% of the exposed surface area becomes buried on binding to the TSLP binding protein. In another embodiment, epitope residues for a particular TSLP binding protein may be identified using the Qt-PISA v2.0.1 software (Protein Interfaces, Complexes and Assemblies; Krissinel and Henrick (2007) as being those residues on full length human TSLP which exhibit an increase in % buried surface area on binding to the TSLP binding protein.

The CDRs L1, L2, L3, H1, H2 and H3 tend to structurally exhibit one of a finite number of main chain conformations. The particular canonical structure class of a CDR is defined by both the length of the CDR and by the loop packing, determined by residues located at key positions in both the CDRs and the framework regions (structurally determining residues or SDRs). Martin and Thornton (1996; J Mol Biol 263:800-815) have generated an automatic method to define the “key residue” canonical templates. Cluster analysis is used to define the canonical classes for sets of CDRs, and canonical templates are then identified by analysing buried hydrophobics, hydrogen-bonding residues, and conserved glycines and prolines. The CDRs of antibody sequences can be assigned to canonical classes by comparing the sequences to the key residue templates and scoring each template using identity or similarity matrices.

Examples of CDR canonicals within the scope of the disclosure are given below. The amino acid numbering used is Kabat.

Examples of canonicals for CDRL1 from SEQ ID NO:9 (e.g., SEQ ID NO:1, 2 or 3) are: Ile 29 is substituted for Val; Leu 33 is substituted for Met, Val, Ile or Phe.

Examples of canonicals for CDRL2 from SEQ ID NO:9 (e.g., SEQ ID NO:4, 5 or 6) are: Ala 51 is substituted for Thr.

Examples of canonicals for CDRL3 from SEQ ID NO:9 (e.g., SEQ ID NO:7 or 8) are: Val 89 is substituted for Gln, Ser, Gly, Phe or Leu; Gln 90 is substituted for Asn or His; Ile 91 is substituted for Phe or Val; Glu 93 is substituted for Ser; Val 96 is substituted for Pro, Tyr, Arg, Ile, Trp or Phe.

There may be multiple variant CDR canonical positions per CDR, per binding unit, per single variable region, and per TSLP binding protein, and therefore any combination of substitutions may be present in the TSLP binding protein of the disclosure, provided that the canonical structure of the CDR is maintained such that the antigen binding protein is capable of specifically binding TSLP.

As discussed above, the particular canonical structure class of a CDR is defined by both the length of the CDR and by the loop packing, determined by residues located at key positions in both the CDRs and the framework regions.

Thus, in addition to the CDRs listed above, the canonical light chain framework residues of a TSLP binding protein of the disclosure may include (using Kabat numbering): Ile, Leu or Val at position 2; Val, Gln, Leu or Glu at position 3; Met or Leu at position 4; Cys at position 23; Leu, Arg or Val at position 46; Tyr, His, Phe, Lys or Trp at position 49; Tyr or Phe at position 71; Cys at position 88; and Phe at position 98.

In one embodiment, the light chain framework comprises the following residues: Ile at position 2, Gln at position 3, Met at position 4, Cys at position 23, Leu at position 46, Trp at position 49, Phe at position 71, Cys at position 88, and Phe at position 98.

Any one, any combination, or all of the framework positions described above may be present in the antigen binding protein of the disclosure. There may be multiple variant framework canonical positions per single variable region and per TSLP binding protein, and therefore any combination may be present in the TSLP binding protein of the disclosure, provided that the canonical structure of the framework is maintained.

“Percent identity” between a query nucleic acid sequence and a subject nucleic acid sequence is the “Identities” value, expressed as a percentage, that is calculated by the BLASTN algorithm when a subject nucleic acid sequence has 100% query coverage with a query nucleic acid sequence after a pair-wise BLASTN alignment is performed. Such pair-wise BLASTN alignments between a query nucleic acid sequence and a subject nucleic acid sequence are performed by using the default settings of the BLASTN algorithm available on the National Center for Biotechnology Institute's website with the filter for low complexity regions turned off. Importantly, a query nucleic acid sequence may be described by a nucleic acid sequence identified in one or more claims herein. In an embodiment, the query amino acid sequence is SEQ ID NO: 10 or SEQ ID NO:11.

“Percent identity” between a query amino acid sequence and a subject amino acid sequence is as defined above. In an embodiment, the query amino acid sequence is SEQ ID NO:9 or SEQ ID NO:12.

The query sequence may be 100% identical to the subject sequence, or it may include up to a certain integer number of amino acid or nucleotide alterations as compared to the subject sequence such that the % identity is less than 100%. For example, the query sequence is at least 50, 60, 70, 75, 80, 85, 90, 95, 96, 97, 98, or 99% identical to the subject sequence. Such alterations include at least one amino acid deletion, substitution (including conservative and non-conservative substitution), or insertion, and wherein said alterations may occur at the amino- or carboxy-terminal positions of the query sequence or anywhere between those terminal positions, interspersed either individually among the amino acids or nucleotides in the query sequence or in one or more contiguous groups within the query sequence. In a particular embodiment a TSLP binding protein of the disclosure is greater than or equal to 95, 96, 97, 98, or 99% identical to SEQ ID NO:9.

The % identity may be determined across the entire length of the query sequence, including the CDR(s). Alternatively, the % identity may exclude the CDR(s), for example the CDR(s) is 100% identical to the subject sequence and the % identity variation is in the remaining portion of the query sequence, so that the CDR sequence is fixed/intact. In a particular embodiment a TSLP binding protein of the disclosure has identical CDRs to the CDRs in SEQ ID NO:9 and has a framework that is 90, 91, 92, 93, 94, 95, 96, 97, 98 or 99% identical to the framework of SEQ ID NO:9.

The variant sequence substantially retains the biological characteristics of the unmodified protein, such as binding to and neutralisation of human and cynomologus monkey TSLP and a lack of IL-7 binding.

The VH or VL sequence may be a variant sequence with up to 10 amino acid substitutions, additions or deletions. For example, the variant sequence may have up to 9, 8, 7, 6, 5, 4, 3, 2 or 1 amino acid substitution(s), addition(s) or deletion(s), e.g., compared with SEQ ID NO:9 or 12. In one embodiment the variant sequence has up to 10 amino acid substitutions, e.g. 9, 8, 7, 6, 5, 4, 3, 2, or 1 amino acid substitutions e.g. compared with SEQ ID NO:9 or 12.

The sequence variation may exclude the CDR(s), for example, the CDR(s) is the same as the VH or VL sequence and the variation is in the remaining portion of the VH or VL sequence, so that the CDR sequence is fixed/intact. In a particular embodiment a TSLP binding protein of the disclosure has identical CDRs to the CDRs in SEQ ID NO:9 and has a framework with up to 10, 9, 8, 7, 6, 5, 4, 3, 2, or 1 amino acid substitution(s), addition(s), or deletion(s) compared with SEQ ID NO:9.

Typically, the variation is a substitution, or a conservative substitution, for example, as shown in Table 1.

The variant sequence substantially retains the biological characteristics of the unmodified protein, such as binding to and neutralisation of human and cynomologus monkey TSLP and a lack of IL-7 binding. The skilled person will appreciate that, upon production of an antigen binding protein such as an antibody, depending on the cell line used, and the particular amino acid sequence of the antigen binding protein, post-translational modifications may occur. For example, this may include the cleavage of certain leader sequences, the addition of various sugar moieties in various glycosylation and phosphorylation patterns, deamidation, oxidation, disulfide bond scrambling, isomerisation, C-terminal lysine clipping, and N-terminal glutamine cyclisation. The present disclosure encompasses the use of antigen binding proteins which have been subjected to, or have undergone, one or more post-translational modifications. Thus an “antigen binding protein” or “antibody” of the disclosure includes an “antigen binding protein” or “antibody”, respectively, as defined earlier which has undergone a post-translational modification such as described herein.

Deamidation is an enzymatic reaction primarily converting asparagine (N) to isoaspartic acid and aspartic acid (D) at approximately 3:1 ratio. To a much lesser degree, deamidation can occur with glutamine residues in a similar manner. Oxidation can occur during production and storage (i.e., in the presence of oxidizing conditions) and results in a covalent modification of a protein, induced either directly by reactive oxygen species or indirectly by reaction with secondary by-products of oxidative stress. Oxidation happens primarily with methionine residues, but occasionally can occur at tryptophan and free cysteine residues.

Disulfide bond scrambling can occur during production and basic storage conditions. Under certain circumstances, disulfide bonds can break or form incorrectly, resulting in unpaired cysteine residues (—SH). These free (unpaired) sulfhydryls (—SH) can promote shuffling.

Isomerization typically occurs during production, purification, and storage (at acidic pH) and usually occurs when aspartic acid is converted to isoaspartic acid through a chemical process.

N-terminal glutamine in the heavy chain and/or light chain is likely to form pyroglutamate (pGlu). Most pGlu formation happens in the production bioreactor, but it can be formed non-enzymatically, depending on pH and temperature of processing and storage conditions. pGlu formation is considered as one of the principal degradation pathways for recombinant mAbs.

C-terminal lysine clipping is an enzymatic reaction catalyzed by carboxypeptidases, and is commonly observed in recombinant mAbs. Variants of this process include removal of lysine from one or both heavy chains. Lysine clipping does not appear to impact bioactivity and has no effect on mAb effector function.

Naturally occurring autoantibodies exist in humans that can bind to proteins. Autoantibodies can thus bind to endogenous proteins (present in naïve subjects) as well as to proteins or peptides which are administered to a subject for treatment. Therapeutic protein-binding autoantibodies and antibodies that are newly formed in response to drug treatment are collectively termed anti-drug antibodies (ADAs). Pre-existing antibodies against molecules such as therapeutic proteins and peptides, administered to a subject can affect their efficacy and could result in administration reactions, hypersensitivity, altered clinical response in treated patients and altered bioavailability by sustaining, eliminating or neutralizing the molecule. It could be advantageous to provide molecules for therapy which comprise human immunoglobulin (antibody) single variable domains or dAbs which have reduced immunogenicity (i.e. reduced ability to bind to pre-existing ADAs when administered to a subject, in particular a human subject).

Thus in one embodiment of the present disclosure there is provided a modified dAb or a polypeptide comprising such a modified dAb, which has reduced ability to bind to pre-existing antibodies (ADAs) as compared to the equivalent unmodified molecule. By reduced ability to bind it is meant that the modified molecule binds with a reduced affinity or reduced avidity to a pre-existing ADA. Said modified dAb comprise one or more C-terminal modifications (addition, extension, deletion or tag).

In one embodiment the modified dAb is a VL dAb and comprises a C-terminal sequence consisting of the sequence VEIKₚR_{q}X; wherein:

p and q each represent 0 or 1 such that when p represents 1 q may be 0 or 1 and such that when p represents 0, q also represents 0;

X may be present or absent, and if present represents an amino acid extension of 1 to 8 amino acids residues, for example a single threonine extension, or a TV, TVA, TVAA, TVAAP, TVAAPS extension;

with the further proviso that if X is absent;

p and/or q is 0, and/or the dAb ending in VEIKₚR_{q}X comprises one or more of said amino acid substitutions.

In an embodiment, the VL dAb comprises amino acids RT at the C-terminus.

In an embodiment, the VL dAb does not comprise amino acid R at the C-terminus.

In one embodiment the modified dAb can comprise a tag present at the C terminus. The tag can be any tag known in the art for example affinity tags such as myc-tags, FLAG tags, his-tags, chemical modification such as PEG, or protein domains such as the antibody Fc domain.

The C terminal addition or extension or tag can be present as a direct fusion or conjugate with the C terminus of the molecule.

The specific immunoassay described in Example 11 can be used to confirm that the modified dAbs have reduced binding to ADAs. dAbs which have reduced binding to ADAs give a reduced luminescence signal in the assay

As discussed above, where inhaled delivery is intended, small size is desirable. However, where other modes of administration are contemplated, binding proteins and anti-TSLP dAbs of the disclosure can be formatted to have a larger hydrodynamic size, for example, by attachment of a PEG group, serum albumin, transferrin, transferrin receptor or, at least, the transferrin-binding portion thereof, an antibody Fc region, or by conjugation to an antibody domain. For example, polypeptides dAbs™ and antagonists may be formatted as a larger antigen-binding fragment of an antibody or as an antibody (e.g., formatted as a Fab, Fab′, F(ab)₂, F(ab′)₂, IgG, scFv).

As used herein, “hydrodynamic size”, refers to the apparent size of a molecule (e.g., an antigen binding protein) based on the diffusion of the molecule through an aqueous solution. The diffusion or motion of a protein through solution can be processed to derive an apparent size of the protein, where the size is given by the “Stokes radius” or “hydrodynamic radius” of the protein particle. The “hydrodynamic size” of a protein depends on both mass and shape (conformation), such that two proteins having the same molecular mass may have differing hydrodynamic sizes based on the overall conformation and charge of the protein. An increase in hydrodynamic size can give an associated decrease in renal clearance leading to an observed increase in half life (t_{1/2}).

Hydrodynamic size of the antigen binding proteins (e.g., domain antibody monomers and multimers) of the disclosure may be determined using methods which are well known in the art. For example, gel filtration chromatography may be used to determine the hydrodynamic size of an antigen binding protein. Examples of gel filtration matrices for determining the hydrodynamic sizes of antigen binding proteins include, cross-linked agarose matrices, which are well known and readily available.

The size of an antigen binding protein format (e.g., the size of a PEG moiety attached to a domain antibody monomer), can vary depending on the desired application. For example, where antigen binding protein is intended to leave the circulation and enter into peripheral tissues, the hydrodynamic size of the antigen binding protein may be kept low to facilitate extravazation from the blood stream. Alternatively, to allow the antigen binding protein remain in the systemic circulation for a longer period of time, the size of the antigen binding protein can be increased, for example, by formatting as an Ig-like protein.

The phrases, “half-life” (“t_{1/2}”) and “serum half life”, refer to the time taken for the serum (or plasma) concentration of an antigen binding protein in accordance with the disclosure to reduce by 50%, in vivo, for example due to degradation of the antigen binding protein and/or clearance or sequestration of the antigen binding protein by natural mechanisms.

Increased half-life, or half-life extension, can be useful in in vivo applications of antigen binding proteins, antibodies, antibody fragments of small size. Such fragments (Fvs, disulphide bonded Fvs, Fabs, scFvs, dAbs) are generally rapidly cleared from the body. Antigen binding proteins in accordance with the disclosure can be adapted or modified to provide increased serum half-life in vivo and consequently longer persistence, or residence, times of the functional activity of the antigen binding protein in the body. Suitably, such modified molecules have a decreased clearance and increased Mean Residence Time compared to the non-adapted molecule. Increased half-life can improve the pharmacokinetic and pharmacodynamic properties of a therapeutic molecule, and can also be important for improved patient compliance.

The antigen binding proteins of the disclosure can be stabilized in vivo and their half-life increased by binding to molecules which resist degradation and/or clearance or sequestration (“half-life extending moiety” or “half-life extending molecule”). Suitable half-life extension strategies include: PEGylation, polysialylation, HESylation, recombinant PEG mimetics, N-glycosylation, O-glycosylation, Fc fusion, engineered Fc, IgG binding, albumin fusion, albumin binding, albumin coupling and nanoparticles.

In one embodiment, the half-life extending moiety or molecule is a polyethylene glycol moiety or a PEG mimetic. In another embodiment, the antigen binding protein comprises (optionally consists of) a single variable domain of the disclosure linked to a polyethylene glycol moiety (optionally, wherein said moiety has a size of about 20 to about 50 kDa, optionally about 40 kDa linear or branched PEG). In yet another embodiment, the antagonist consists of a domain antibody monomer linked to a PEG, wherein the domain antibody monomer is a single variable domain according to the disclosure.

The interaction between the Fc region of an antibody and various Fc receptors (FcγR) is believed to mediate phagocytosis and half-life/clearance of an antibody or antibody fragment. The neonatal FcRn receptor is believed to be involved in both antibody clearance and the transcytosis across tissues. In one embodiment, the half-life extending moiety may be an Fc region from an antibody. Such an Fc region may incorporate various modifications depending on the desired property. For example, a salvage receptor binding epitope may be incorporated into the antibody to increase serum half life.

Human IgG1 residues determined to interact directly with human FcRn includes Ile253, Ser254, Lys288, Thr307, Gln311, Asn434 and His435. Accordingly, substitutions at any of the positions described in this section may enable increased serum half-life and/or altered effector properties of the antibodies.

Typically, a polypeptide that enhances serum half-life in vivo, i.e. a half-life extending molecule, is a polypeptide which occurs naturally in vivo and which resists degradation or removal by endogenous mechanisms which remove unwanted material from the organism (e.g., human). Typically, such molecules are naturally occurring proteins which, themselves, have a long half-life in vivo.

For example, a polypeptide that enhances serum half-life in vivo can be: proteins from the extracellular matrix, proteins found in blood, proteins found at the blood brain barrier or in neural tissue, proteins localized to the kidney, liver, muscle, lung, heart, skin or bone, stress proteins, disease-specific proteins, or proteins involved in Fc transport.

Such an approach can also be used for targeted delivery of an antigen binding protein, e.g., a single variable domain, in accordance with the disclosure to a tissue of interest. In one embodiment, targeted delivery of a high affinity single variable domain in accordance with the disclosure is provided.

In one embodiment, an antigen binding protein, e.g., single variable domain, in accordance with the disclosure can be linked, i.e. conjugated or associated, to serum albumin, fragments and analogues thereof.

In another embodiment, a single variable domain, polypeptide or ligand in accordance with the disclosure can be linked, i.e., conjugated or associated, to transferrin, fragments and analogues thereof.

In another embodiment, half-life extension can be achieved by targeting an antigen or epitope that increases half-live in vivo. The hydrodynamic size of an antigen binding protein and its serum half-life may be increased by conjugating or associating an antigen binding protein of the disclosure to a binding domain that binds a naturally occurring molecule and increases half-live in vivo.

For example, the antigen binding protein in accordance with the disclosure can be conjugated or linked to an anti-serum albumin (SA) or anti-neonatal Fc receptor antibody or antibody fragment, e.g., an anti-SA or anti-neonatal Fc receptor dAb, Fab, Fab′ or scFv, or to an anti-SA affibody or anti-neonatal Fc receptor Affibody or an anti-SA avimer, or an anti-SA binding domain which comprises a scaffold selected from, but not limited to, the group consisting of: CTLA-4, lipocallin, SpA, an affibody, an avimer, GroEl and fibronectin. Conjugating refers to a composition comprising polypeptide, dAb or antagonist of the disclosure that is bonded (covalently or noncovalently) to a binding domain such as a binding domain that binds serum albumin.

In another embodiment, the binding domain may be a polypeptide domain, such as an Albumin Binding Domain (ABD), or a small molecule which binds albumin.

One embodiment provides a fusion protein comprising an antigen binding protein in accordance with the disclosure and an anti-serum albumin or anti-neonatal Fc receptor antibody or antibody fragment.

In one embodiment, a single variable domain of the present disclosure is identified to be preferentially monomeric. Another embodiment provides a (substantially) pure monomer. In yet another embodiment, the single variable domain is at least 65%, 70%, 75%, 80%, 85%, 90%, 95%, 98%, 99% pure or 100% pure monomer. To determine whether single variable domains are monomeric or form higher order oligomers in solution, they can be analysed by SEC-MALLS. SEC MALLS (size exclusion chromatography with multi-angle-LASER-light-scattering) is a non-invasive technique for the characterization of macromolecules in solution, that is familiar to any skilled in the art. Briefly, proteins (at concentration of 1 mg/mL in buffer Dulbecco's PBS) are separated according to their hydrodynamic properties by size exclusion chromatography (column: TSK3000; S200). Following separation, the propensity of the protein to scatter light is measured using a multi-angle-LASER-light-scattering (MALLS) detector. The intensity of the scattered light while protein passes through the detector is measured as a function of angle. This measurement taken together with the protein concentration determined using the refractive index (RI) detector allows calculation of the molar mass using appropriate equations (integral part of the analysis software Astra v. 5.3.4.12).

In one embodiment, a single variable domain of the present disclosure is thermally stable. In another embodiment a single variable domain of the present disclosure has a Tm of greater than or equal to 50° C. as measured by DSC with a scanning speed of 3° C. per min and a protein concentration of 1 mg/ml.

Antigen binding proteins may be prepared by any of a number of conventional techniques. For example, antigen binding proteins may be purified from cells that naturally express them (e.g., an antibody can be purified from a hybridoma that produces it), or produced in recombinant expression systems.

A number of different expression systems and purification regimes can be used to generate the antigen binding protein of the disclosure. Generally, host cells are transformed with a recombinant expression vector encoding the desired antigen binding protein. A wide range of host cells can be employed, including Prokaryotes (including Gram negative or Gram positive bacteria, for example Escherichia coli, Bacilli sp., Pseudomonas sp., Corynebacterium sp.), Eukaryotes including yeast (for example Saccharomyces cerevisiae, Pichia pastoris), fungi (for example Aspergillus sp.), or higher Eukaryotes including insect cells and cell lines of mammalian origin (for example, CHO, Perc6, HEK293, HeLa).

The host cell may be an isolated host cell. The host cell is usually not part of a multicellular organism (e.g., plant or animal). The host cell may be a non-human host cell.

Appropriate cloning and expression vectors for use with bacterial, fungal, yeast, and mammalian cellular hosts and methods of cloning are known in the art.

The cells can be cultured under conditions that promote expression of the antigen binding protein, and the polypeptide recovered by conventional protein purification procedures. The antigen binding proteins contemplated for use herein include substantially homogeneous antigen binding proteins substantially free of contaminating materials.

The skilled person will appreciate that, upon production of the antigen binding protein, in particular depending on the cell line used and particular amino acid sequence of the antigen binding protein, post-translational modifications may occur. Such post-translational modifications may include the cleavage of certain leader sequences, the addition of various sugar moieties in various glycosylation patterns, deamidation (for example at an asparagine or glutamine residue), oxidation (for example at a methionine, tryptophan or free cysteine residue), disulfide bond scrambling, isomerisation (for example at an aspartic acid residue), C-terminal lysine clipping (for example from one or both heavy chains), and N-terminal glutamine cyclisation (for example, in the heavy and/or light chain). The present disclosure encompasses the use of antibodies which have been subjected to, or have undergone, one or more post-translational modifications. The modification may occur in a CDR, the variable framework region, or the constant region. The modification may result in a change in charge of the molecule.

Antigen binding protein as described herein may be incorporated into pharmaceutical compositions for use in the treatment of the human diseases described herein. In one embodiment, the pharmaceutical composition comprises an antigen binding protein as disclosed herein, for example a single variable domain of SEQ ID NO:9 or 12, optionally in combination with one or more pharmaceutically acceptable carriers and/or excipients. In a further embodiment, the pharmaceutical composition comprises a TSLP binding protein of the present invention, for example a single variable domain of SEQ ID NO:9 or 12, and a pharmaceutically acceptable carrier or excipient.

Such compositions comprise a pharmaceutically acceptable carrier as known and called for by acceptable pharmaceutical practice.

Pharmaceutical compositions may be administered by injection or continuous infusion (examples include, but are not limited to, intravenous, intraperitoneal, intradermal, subcutaneous, intramuscular and intraportal). Pharmaceutical compositions may be suitable for topical administration (which includes, but is not limited to, epicutaneous, inhaled, intranasal or ocular administration) or enteral administration (which includes, but is not limited to, oral or rectal administration). In one embodiment, the pharmaceutical composition is inhaled. Pharmaceutical compositions may comprise between 0.3 μg to 100 mg of TSLP binding protein, for example between 1 μg to 30 mg of TSLP binding protein. In one embodiment, the pharmaceutical composition contains between 2 mg to 50 mg, for example 2 mg, 5 mg, 15 mg and 50 mg. Alternatively, the composition may comprise between 1 μg and 15 mg, for example between 1 μg and 10 mg. In an embodiment the pharmaceutical composition comprises between 250 μg and 5 mg, for example 500 μg and 2.5 mg of TSLP binding protein.

Methods for the preparation of such pharmaceutical compositions are well known to those skilled in the art. Other excipients may be added to the composition as appropriate for the mode of administration and the particular protein used. Examples of different excipients and their uses are described in Lowe et al., Adv Protein Chem Struct Biol, 84, 41-61 (2011).

Effective doses and treatment regimes for administering the TSLP binding protein may be dependent on factors such as the age, weight and health status of the patient and disease to be treated. Such factors are within the purview of the attending physician. Guidance in selecting appropriate doses may be found in e.g Bai et al., Clin Pharmacokinet, 51, 119-35 (2012).

The pharmaceutical composition may comprise a kit of parts of the TSLP binding protein together with other medicaments, optionally with instructions for use. For convenience, the kit may comprise the reagents in predetermined amounts with instructions for use.

In an embodiment of the disclosure, a TSLP binding protein, in particular an anti-TSLP single variable domain, is directed to a dosage form adapted for administration to a patient by inhalation, for example as a dry powder, an aerosol, a suspension, or a solution composition. In one embodiment, the disclosure is directed to a dosage form adapted for administration to a patient by inhalation as a dry powder. In a further embodiment, the present invention provides a dosage form adapted for administration to a patient by inhalation via a nebulizer.

The formulations of the invention may be buffered by the addition of suitable buffering agents.

Dry powder compositions for delivery to the lung by inhalation typically comprise a TSLP binding protein as a finely divided powder which may be together with one or more pharmaceutically-acceptable excipients as separate finely divided powders or within the same powder particle as the TSLP binding protein. Pharmaceutically-acceptable excipients suited for use in dry powders are known to those skilled in the art and include lactose, starch, mannitol, mono-, di-, and polysaccharides, amino acids or small peptides, salts or mono- or di-valent cations and lipids. The finely divided powder may be prepared by, for example, micronisation milling or by direct particle formation methods such as spray-drying, PRINT™ (Liquidia), or supercritical fluid precipitation. Generally, the finely divided powder consists of particles of the protein or particles containing the protein that can be defined by a D50 value of about 1 to about 10 microns (for example as measured using laser diffraction).

The dry powder may be administered to the patient via a reservoir dry powder inhaler (RDPI) having a reservoir suitable for storing multiple (un-metered doses) of medicament in dry powder form. RDPIs typically include a means for metering each medicament dose from the reservoir to a delivery position. For example, the metering means may comprise a metering cup, which is movable from a first position where the cup may be filled with medicament from the reservoir to a second position where the metered medicament dose is made available to the patient for inhalation.

Alternatively, the dry powder may be presented in capsules (e.g., gelatin or plastic), cartridges, or blister packs for use in a multi-dose dry powder inhaler (MDPI). MDPIs are inhalers wherein the medicament is comprised within a multi-dose pack containing (or otherwise carrying) multiple defined doses (or parts thereof) of medicament. When the dry powder is presented as a blister pack, it comprises multiple blisters for containment of the medicament in dry powder form. The blisters are typically arranged in regular fashion for ease of release of the medicament therefrom. For example, the blisters may be arranged in a generally circular fashion on a disc-form blister pack, or the blisters may be elongate in form, for example comprising a strip or a tape. Each capsule, cartridge, or blister may, for example, contain between 15 μg-10 mg of the TSLP binding protein. Suitable examples of MDPIs include, without limitation, those exemplified, as in Diskus™, see GB2242134, U.S. Pat. Nos. 6,032,666, 5,860,419, 5,873,360, 5,590,645, 6,378,519 and 6,536,427 or Diskhaler, see GB 2178965, 2129691 and 2169265, U.S. Pat. Nos. 4,778,054, 4,811,731, 5,035,237, as well as metered in use (e.g. as in Turbuhaler, see EP 0069715, or in the devices described in U.S. Pat. No. 6,321,747). An example of a unit-dose device that may be used is Rotahaler (see GB 2064336). Other suitable MDIs include, without limitation, those pertaining to twin-blister strip devices, e.g., the Ellipta™ device (see e.g., U.S. Pat. Nos. 8,113,199; 8,161,968; 8,511,304; 8,534,281 and 8,746,242).

Capsules and cartridges for use in an inhaler or insufflator, of for example gelatine, may be formulated containing a powder mix for inhalation of a TSLP binding protein or a particulate formulation of the TSLP binding protein with one or more excipients and a suitable powder base such as lactose, mannitol or starch. Each capsule or cartridge may generally contain from 15 μg to 10 mg of the TSLP binding protein or a particulate formulation of the TSLP binding protein with one or more excipients. In one embodiment, the capsule or cartridge contains between 2 mg to 50 mg of the TSLP binding protein or a particulate formulation of the TSLP binding protein with one or more excipients, for example 2 mg, 5 mg, 15 mg and 50 mg. Alternatively, the TSLP binding protein or a particulate formulation of the TSLP binding protein with one or more excipients may be presented without further powder base excipients such as lactose, mannitol or starch.

The proportion of the TSLP binding protein in the disclosed local compositions depends on the precise type of formulation to be prepared, but will generally be within the range of from 0.001 to 100% by weight. Generally, for most types of preparations, the proportion used will be within the range of from 0.005 to 90%, for example from 0.01 to 80%.

In one embodiment of the invention, the overall daily dose and the metered dose delivered by blisters in an MDPI are arranged so that each metered dose contains from 15 μg to 13 mg, from 20 μg to 2000 μg, or from 500 μg to 1500 μg of a TSLP binding protein. Administration may be once daily or several times daily, for example 2, 3, 4 or 8 times, giving for example 1, 2 or 3 doses each time. The overall daily dose with an aerosol will be within the range from 100 μg to 20 mg, or from 200 μg to 2000 μg. The overall daily dose and the metered dose delivered by capsules and cartridges in an inhaler or insufflator will generally be up to treble that delivered with MDPIs.

Suspensions and solutions comprising a TSLP binding protein may also be administered to a patient via a nebulizer. The solvent or suspension agent utilized for nebulization may be any pharmaceutically-acceptable liquid such as water, aqueous saline, alcohols or glycols, e.g., ethanol, isopropylalcohol, glycerol, propylene glycol, polyethylene glycol, etc. or mixtures thereof. Saline solutions utilize salts which display little or no pharmacological activity after administration. Both organic salts, such as alkali metal or ammonium halogen salts, e.g., sodium chloride, potassium chloride or organic salts, such as potassium, sodium and ammonium salts or organic acids, e.g., ascorbic acid, citric acid, acetic acid, tartaric acid, etc., may be used for this purpose.

Other pharmaceutically-acceptable excipients may be added to the suspension or solution. The TSLP binding protein may be stabilized by the addition of an inorganic acid, e.g., hydrochloric acid, nitric acid, sulphuric acid and/or phosphoric acid; an organic acid, e.g., ascorbic acid, citric acid, acetic acid, and tartaric acid, etc., a complexing agent such as EDTA or citric acid and salts thereof; or an antioxidant such as vitamin E or ascorbic acid or an amino acid based antioxidant such as methionine. These inorganic acids may be used alone or together to stabilize the TSLP binding protein. Preservatives may be added such as benzalkonium chloride or benzoic acid and salts thereof. Surfactant may be added to improve the physical stability of suspensions. These include lecithin, disodium dioctylsulphosuccinate, oleic acid and sorbitan esters.

The terms “individual”, “subject” and “patient” are used herein interchangeably. In one embodiment, the subject is a mammal, such as a primate, for example a marmoset or monkey. In another embodiment, the subject is a human.

The TSLP binding protein described herein may also be used in methods of treatment. Treatment can be therapeutic, prophylactic or preventative. Treatment encompasses alleviation, reduction, or prevention of at least one aspect or symptom of a disease and encompasses prevention or cure of the diseases described herein.

The TSLP binding protein described herein is used in an effective amount for therapeutic, prophylactic or preventative treatment. A therapeutically effective amount of the antigen binding protein described herein is an amount effective to ameliorate or reduce one or more symptoms of, or to prevent or cure, a disease.

A TSLP binding protein described herein may be used as a medicament, in particular for use in treating any one of the following disorders.

A TSLP binding protein described herein may be used in the manufacture of a medicament for use in treating any one of the following disorders.

TSLP expression and/or function is linked to a number of inflammatory disorders, predominantly those allergic in nature (characterised by immunoglobulin E (IgE)-related immunological responses), but also non-allergic diseases. These diseases include, but are not limited to, asthma (including severe asthma), idiopathic pulmonary fibrosis, atopic dermatitis (AD), allergic conjunctivitis, allergic rhinitis (AR), Netherton syndrome (NS), eosinophilic esophagitis (EoE), food allergy, allergic diarrhoea, eosinophilic gastroenteritis, allergic bronchopulmonary aspergillosis (ABPA), allergic fungal sinusitis, cancer (e.g., breast, pancreas, B-cell acute lymphoblastic leukaemia), rheumatoid arthritis, COPD, systemic sclerosis, keloids, ulcerative colitis, chronic rhinosinusitis (CRS), and nasal polyposis. In addition as TSLP stimulates the production of the type 2 cytokines IL-5, IL-13 and IL-4, it is also implicated in diseases to which these cytokines have been linked, such as, but not limited to asthma, allergic rhinitis, chronic eosinophilic pneumonia, eosinophilic bronchitis, allergic bronchopulmonary aspergillosis, coeliac disease, eosinophilic gastroenteritis, Churg-Strauss syndrome, eosinophilic myalgia syndrome, hypereosinophilic syndrome, eosinophilic granulomatosis with polyangiitis, eosinophilic esophagitis, and inflammatory bowel disease.

Accordingly, in one embodiment, the invention provides a TSLP binding protein as described herein for use in treating a disease associated with TSLP signalling. Use of a TSLP binding protein as defined herein in the manufacture of a medicament for the treatment of a disease associated with TSLP signalling is also provided. The invention provides a method of treating a disease associated with TSLP signalling in a human patient in need thereof, the method comprising administering a TSLP binding protein as defined herein to the human patient.

In one embodiment, the disease associated with TSLP signalling is selected from the group consisting of: asthma, idiopathic pulmonary fibrosis, atopic dermatitis, allergic conjunctivitis, allergic rhinitis, Netherton syndrome, eosinophilic esophagitis (EoE), food allergy, allergic diarrhoea, eosinophilic gastroenteritis, allergic bronchopulmonary aspergillosis (ABPA), allergic fungal sinusitis, cancer, rheumatoid arthritis, COPD, systemic sclerosis, keloids, ulcerative colitis, chronic rhinosinusitis (CRS), nasal polyposis, chronic eosinophilic pneumonia, eosinophilic bronchitis, coeliac disease, Churg-Strauss syndrome, eosinophilic myalgia syndrome, hypereosinophilic syndrome, eosinophilic granulomatosis with polyangiitis and inflammatory bowel disease. In a more particular embodiment, the disease associated with TSLP signalling is selected from the group consisting of: asthma, idiopathic pulmonary fibrosis, atopic dermatitis, allergic conjunctivitis, allergic rhinitis, Netherton syndrome, eosinophilic esophagitis (EoE), food allergy, allergic diarrhoea, eosinophilic gastroenteritis, allergic bronchopulmonary aspergillosis (ABPA), allergic fungal sinusitis, cancer, rheumatoid arthritis, COPD, systemic sclerosis, keloids, ulcerative colitis, chronic rhinosinusitis (CRS) and nasal polyposis. In an even more particular embodiment, the disease associated with TSLP signalling is asthma.

Asthma is a common chronic inflammatory disease of the airways characterized by variable and recurring symptoms, reversible airflow obstruction and bronchospasm. Common symptoms include wheezing, coughing, chest tightness, and shortness of breath. Asthma is thought to be caused by a combination of genetic and environmental factors and is managed largely by the use of bronchodilators and inhaled or oral corticosteroids.

Inhaled corticosteroids include fluticasone propionate, fluticasone furoate, beclomethasone diproprionate, budesonide, ciclesonide, mometasone furoate, triamcinolone and flunisolide.

Bronchodilators include β2-adrenoreceptor agonists and muscarinic antagonists. Example β2-adrenoreceptor agonists include vilanterol, salmeterol, salbutamol, formoterol, salmefamol, fenoterol carmoterol, etanterol, naminterol, clenbuterol, pirbuterol, flerbuterol, reproterol, bambuterol, indacaterol, terbutaline and salts thereof, for example the xinafoate (1-hydroxy-2-naphthalenecarboxylate) salt of salmeterol, the sulphate salt or free base of salbutamol, the fumarate salt of formoterol, or the trifenatate salt of vilanterol. Example muscarinic antagonists include umeclidinium, tiotropium, glycopyrrolate, ipratropium, and salts thereof such as the bromide salt of umeclidinium (umeclidinium bromide).

Severe asthma is asthma which requires treatment with guidelines suggested medications for GINA steps 4-5 asthma (high dose inhaled corticosteroid (CS) and LABA or leukotriene modifier/theophylline) for the previous year or systemic CS for >50% of the previous year to prevent it from becoming “uncontrolled” or which remains “uncontrolled” despite this therapy.

COPD is a progressive lung diseases most often associated with smoking and characterised by chronic bronchitis and emphysema. Declining lung function, dyspnoea, mucus overproduction and cough are the hallmark features of the disease. COPD is managed largely pharmacologically by bronchodilators and steroids and by oxygen therapy.

Atopic dermatitis (AD) is characterized by chronic and relapsing inflammatory eczematous disease of the skin characterized by skin lesions, elevated serum total IgE and exaggerated Th2 (Leung et al. Current Opinion in Immunology 15(6):634-638 (2003)) responses resulting in high levels of IL-4, IL-5 and IL-13. The triggers for AD are not well understood but include a combination of genetic factors and also environmental factors which may act as allergens.

Eosinophilic esophagitis (EoE) is a chronic inflammatory disease characterized by eosinophilic infiltration of the esophageal mucosa (Roman et al. Digestive and Liver Disease 45(11):871-878 (2013)). EoE affects both adults and children and is associated with esophageal narrowings and often presents with food impaction, dysphagia, poor weight gain, vomiting and decreased appetite. Topical viscous corticosteroids or diet elimination are the treatment of choice.

Netherton syndrome (NS) is a severe skin disease characterized by AD-like lesions, as well as other allergic manifestations that result from mutations in the SPINK5 gene, which encodes the serine protease inhibitor LEKTI. TSLP is strongly expressed in the skin of individuals with NS.

A TSLP binding protein of the invention may be administered alone or in combination with other therapeutic agents. The TSLP binding protein and one or more other therapeutic agents may be administered separately, simultaneously or sequentially.

A TSLP binding protein of the invention may be administered in combination with inhaled, intranasal or parenteral corticosteroids such fluticasone furoate, fluticasone propionate, budesonide, ciclesonide, beclomethasone dipropionate, mometasone furoate, triamcinolone acetonide and prednisolone. In one embodiment, a TSLP binding protein of the invention may be administered as a fixed dose combination with an inhaled corticosteroid such as a fixed dose combination with fluticasone furoate or fluticasone propionate.

A TSLP binding protein of the present invention may be administered in combination with a bronchodilator such as a beta-2 adrenoreceptor agonist and/or a muscarinic antagonist. Suitable beta-2 adrenoreceptor agonists include vilanterol, salmeterol, salbutamol, formoterol, salmefamol, fenoterol, carmoterol, etanterol, naminterol, clenbuterol, pirbuterol, flerbuterol, reproterol, bambuterol, indacaterol, terbutaline, and salts thereof. Suitable muscarinic antagonists include umeclidinium, tiotropium, glycopyrrolate, ipratropium, and salts thereof such as the bromide salt of umeclidinium. In one embodiment, a TSLP binding protein of the invention may be administered as a fixed dose combination with a beta-2 adrenoreceptor agonist and/or a muscarinic antagonist such as a fixed dose combination with vilanterol trifenatate, umeclidinium bromide, or the dual combination of vilanterol trifenatate and umeclidinium bromide.

A TSLP binding protein of the present invention may be administered with a combination of one or more bronchodilators and an inhaled steroid. Such combinations may include dual combinations such as fluticasone furoate and vilanterol trifenatate, fluticasone furoate and umeclidinium bromide, fluticasone propionate and salmeterol, budesonide and formoterol, mometasone and formoterol, and triple therapy such as fluticasone furoate, vilanterol trifenatate and umeclidinium bromide. In one embodiment, a TSLP binding protein of the present invention may be administered as a fixed dose combination with an inhaled corticosteroid and one or more bronchodilators, such as a fixed dose combination with fluticasone furoate and vilanterol trifenatate, or fluticasone propionate and salmeterol, or fluticasone furoate and umeclidinium bromide, or fluticasone furoate, vilanterol trifenatate and umeclidinium bromide.

A TSLP binding protein of the present invention may be administered in combination with anti-leukotriene antagonists such as montelukast, zafirlukast and pranlukast; PDE4 inhibitors such as roflumilast; xanthenes; anti-IgE antibodies such as omalizumab; antagonists of IL-5 such as mepolizumab, benralizumab and reslizumab; antagonists of IL-13 such as lebrikizumab and tralokinumab; antagonists of IL-4/IL-13 such as dupilumab; antagonists of IL-6 such as sirukumab and antagonists of IL-1, IL-4, IL-33, IL-25 and TNF-α.

A TSLP binding protein of the present invention may be administered in combination with an anti-histamine such as cetirizine hydrochloride, levocetirizine, desloratidine, loratidine, fexofenadine hydrochloride or azelastine.

A TSLP binding protein of the present invention may be administered in combination with pirfenidone or nintedanib or an avB6 antagonist, for example, those disclosed in WO2014/154725.

Particular embodiments of the invention include the following:

### Embodiment 1

A TSLP binding protein that comprises the following CDRs: CDR1, CDR2 and CDR3 from SEQ ID NO:9 or a variant of any one or all of these CDRs, wherein the TSLP binding protein binds to TSLP with a dissociation constant (KD) of less that 2 nM and/or competes for binding to TSLP with a single variable domain of SEQ ID NO:9.

### Embodiment 2

A TSLP binding protein that comprises:

(i) CDR1 according to SEQ ID NO:1 or a variant of SEQ ID NO:1 wherein Pro 28 is substituted for Asn, Ser, Asp, Thr or Glu; Ile 29 is substituted for Val; Arg 30 is substituted for Asp, Leu, Tyr, Val, Ile, Ser, Asn, Phe, His, Gly or Thr; Asn 31 is substituted for Ser, Thr, Lys or Gly; Trp 32 is substituted for Phe, Tyr, Asn, Ala, His, Ser or Arg; Leu 33 is substituted for Met, Val, Ile or Phe; Asp 34 is substituted for Ala, Gly, Asn, Ser, His, Val or Phe,
(ii) CDR2 according to SEQ ID NO:4 or a variant of SEQ ID NO:4, wherein Ala 51 is substituted for Thr, Gly or Val, and
(iii) CDR3 according to SEQ ID NO:7 or a variant of SEQ ID NO:7, wherein Val 89 is substituted for Gln, Ser, Gly, Phe or Leu; Gln 90 is substituted for Asn or His; Ile 91 is substituted for Asn, Phe, Gly, Ser, Arg, Asp, His, Thr, Tyr or Val; Gly 92 is substituted for Asn, Tyr, Trp, Thr, Ser, Arg, Gln, His, Ala or Asp; Glu 93 is substituted for Asn, Gly, His, Thr, Ser, Arg, or Ala; Asp 94 is substituted for Tyr, Thr, Val, Leu, His, Asn, Ile, Trp, Pro or Ser; Val 96 is substituted for Pro, Leu, Tyr, Arg, Ile, Trp or Phe; and wherein the TSLP binding protein binds to TSLP with a dissociation constant (KD) of less that 2 nM and/or competes for binding to TSLP with a single variable domain of SEQ ID NO:9.

### Embodiment 3

A TSLP binding protein according to embodiments 1 or 2, wherein CDR1 consists of SEQ ID NO:1; CDR2 consists of SEQ ID NO:4; and/or CDR3 consists of SEQ ID NO:7.

### Embodiment 4

A TSLP binding protein of any one of embodiments 1 to 3 that comprises a light chain framework comprising the following residues: Ile, Leu or Val at position 2; Val, Gln, Leu or Glu at position 3; Met or Leu at position 4; Cys at position 23; Trp at position 35; Tyr, Leu or Phe at position 36; Leu, Arg or Val at position 46; Tyr, His, Phe, Lys or Trp at position 49; Tyr or Phe at position 71; Cys at position 88; and Phe at position 98.

### Embodiment 5

The TSLP binding protein of embodiment 4, wherein the light chain framework comprises the following residues: Ile at position 2, Gln at position 3, Met at position 4, Cys at position 23, Trp at position 35, Tyr at position 36, Leu at position 46, Trp at position 49, Phe at position 71, Cys at position 88, and Phe at position 98.

### Embodiment 6

The TSLP binding protein of any one of embodiments 1 to 5, wherein the TSLP binding protein is a single variable domain.

### Embodiment 7

The TSLP binding protein of embodiment 6, wherein the single variable domain is a V_{K} single variable domain.

### Embodiment 8

The TSLP binding protein of embodiment 7, wherein the V_{K} single variable domain has a C-terminus ending in RT.

### Embodiment 9

The TSLP binding protein of embodiment 7, wherein the V_{K} single variable domain has a C-terminus that does not end in R.

### Embodiment 10

The TSLP binding protein of embodiment 5 comprising a single variable domain of SEQ ID NO:9.

### Embodiment 11

An anti-TSLP single variable domain comprising an amino acid sequence according to SEQ ID NO:9, having up to 10 amino acid substitutions, deletions or additions, in any combination that binds to TSLP with a dissociation constant (KD) of less than 2 nM and/or competes for binding to TSLP with a single variable domain of SEQ ID NO:9.

### Embodiment 12

An anti-TSLP single variable domain according to embodiment 11, wherein the said amino acid substitutions, deletions or additions are not within CDR3.

### Embodiment 13

An anti-TSLP single variable domain according to embodiment 12, wherein the said amino acid substitutions, deletions or additions are not within any of the CDRs.

### Embodiment 14

An anti-TSLP single variable domain consisting of an amino acid sequence according to SEQ ID NO:9.

### Embodiment 15

An isolated polypeptide comprising an anti-TSLP single variable domain as claimed in any one of embodiments 9-14, wherein said isolated polypeptide binds to TSLP.

### Embodiment 16

A TSLP binding protein as claimed in any one of embodiments 1-10, an anti-TSLP single variable domain according to any one of embodiments 11-14, or a polypeptide according to embodiment 15, wherein said TSLP binding protein, anti-TSLP single variable domain or polypeptide binds to human TSLP.

### Embodiment 17

A TSLP binding protein, an anti-TSLP single variable domain or polypeptide according to embodiment 16, wherein said TSLP binding protein, anti-TSLP single variable domain or polypeptide also binds to cynomologus TSLP.

### Embodiment 18

A TSLP binding protein, an anti-TSLP single variable domain or polypeptide according to embodiment 16, wherein said TSLP binding protein, anti-TSLP single variable domain or polypeptide neutralises TSLP activity.

### Embodiment 19

A TSLP binding protein, an anti-TSLP single variable domain or polypeptide according to embodiment 18, wherein said TSLP binding protein, anti-TSLP single variable domain or polypeptide inhibits binding of TSLP to the TSLP receptor.

### Embodiment 20

An isolated nucleic acid encoding a TSLP binding protein, an anti-TSLP single variable domain or polypeptide according to any one of embodiments 1-19.

### Embodiment 21

An isolated nucleic acid molecule according to embodiment 20, comprising SEQ ID NO:10 or SEQ ID NO:11.

### Embodiment 22

A vector comprising a nucleic acid molecule according to embodiment 20 or embodiment 21.

### Embodiment 23

A host cell comprising a nucleic acid according to embodiment 20 or 21 or a vector according to embodiment 22.

### Embodiment 24

A method of producing a polypeptide comprising a TSLP binding protein according to any one of embodiments 1-10, an anti-TSLP single variable domain according to any one of embodiments 11-14, or a polypeptide according to embodiment 15, the method comprising maintaining a host cell according to embodiment 23 under conditions suitable for expression of said nucleic acid or vector, whereby a polypeptide comprising a TSLP binding protein or single variable domain is produced.

### Embodiment 25

A TSLP binding protein according to any one of embodiments 1-10, an anti-TSLP single variable domain according to any one of embodiments 11-14, or a polypeptide according to embodiment 15 for use as a medicament.

### Embodiment 26

A pharmaceutical composition comprising a TSLP binding protein according to any one of embodiments 1-10, an anti-TSLP single variable domain according to any one of embodiments 11-14, or a polypeptide according to embodiment 15.

### Embodiment 27

A TSLP binding protein according to any one of embodiments 1-10, an anti-TSLP single variable domain according to any one of embodiments 11-14, a polypeptide according to embodiment 15, or a pharmaceutical composition according to embodiment 26 for treatment of a disease associated with TSLP signalling.

### Embodiment 28

Use of a TSLP binding protein according to any one of embodiments 1-10, an anti-TSLP single variable domain according to any one of embodiments 11-14 or a polypeptide according to embodiment 15 in the manufacture of a medicament for the treatment of a disease associated with TSLP signalling.

### Embodiment 29

A TSLP binding protein, an anti-TSLP single variable domain, a polypeptide or a pharmaceutical composition according to embodiment 27, or use according to embodiment 28, wherein the disease associated with TSLP signalling is selected from the group consisting of: asthma, atopic dermatitis, allergic conjunctivitis, allergic rhinitis, Netherton syndrome, eosinophilic esophagitis (EoE), allergic diarrhoea, allergic bronchopulmonary aspegillosis (ABPA), allergic fungal sinusitis, cancer, rheumatoid arthritis, COPD, systemic sclerosis, chronic rhinosinusitis (CRS), and nasal polyposis.

### Embodiment 30

A method of treating a TSLP-mediated condition in a human patient in need thereof, the method comprising the step of: administering a composition comprising a TSLP binding protein according to any one of embodiments 1-10, an anti-TSLP single variable domain according any one of embodiments 11-14, or a polypeptide according to embodiment 15 to the human patient.

### Embodiment 31

A kit comprising a TSLP binding protein according to any one of embodiments 1-10, an anti-TSLP single variable domain according to any one of embodiments 11-14, or a polypeptide according to embodiment 15 and a device for inhaling said TSLP binding protein, single variable domain or polypeptide.

Further particular embodiments of the invention include the following:

### Embodiment 1

A TSLP binding protein that comprises an amino acid sequence selected from the group consisting of:
a. CDR1, CDR2, and CDR3 of SEQ ID NO: 9 or a variant thereof, wherein the CDR variant has between 1 and 3 amino acid modifications; and
b. an amino acid sequence at least 90% identical to the sequence of SEQ ID NO: 9;
   wherein the TSLP binding protein has an IC50 of less than or equal to 5 nM.

### Embodiment 2

The TSLP binding protein according to embodiment 1, wherein in a variant of CDR1, the residue corresponding to residue 28 in SEQ ID NO:9 is Pro, the residue corresponding to residue 30 in SEQ ID NO:9 is Arg, the residue corresponding to residue 31 in SEQ ID NO:9 is Asn, the residue corresponding to residue 32 in SEQ ID NO: 9 is Trp, and the residue corresponding to residue 34 in SEQ ID NO:9 is Asp; in a variant of CDR2, the residue corresponding to residue 50 in SEQ ID NO:9 is Gly, the residue corresponding to residue 53 in SEQ ID NO:9 is His, and the residue corresponding to residue 55 in SEQ ID NO:9 is Gln; and in a variant of CDR3 the residue corresponding to residue 91 in SEQ ID NO:9 is Ile, Leu, Val or Phe, the residue corresponding to residue 92 in SEQ ID NO:9 is Gly or Ala, the residue corresponding to residue 93 in SEQ ID NO:9 is Glu, Phe, Asp or Ser, and the residue corresponding to residue 94 in SEQ ID NO:9 is Asp.

### Embodiment 3

The TSLP binding protein according to embodiment 2, wherein in a variant of CDR2, the residue corresponding to residue 46 in SEQ ID NO:9 is Leu.

### Embodiment 4

The TSLP binding protein according to embodiment 2, wherein CDR3 consists of the sequence X₁GlnX₂X₃X₄AspProX₅Thr, wherein X₁ represents Lys, Trp, Val, Met or Ile, X₂ represents Val, Leu, Ile or Phe, X₃ represents Gly or Ala, X₄ represents Glu, Phe, Asp or Ser, and X₅ represents Val or Thr.

### Embodiment 5

The TSLP binding protein according to embodiment 2, wherein the TSLP binding protein exhibits less than or equal to a 5-fold difference in IC50 using human and cynomolgus TSLP, and wherein the residue corresponding to residue 91 in SEQ ID NO:9 is Ile, Leu or Val.

### Embodiment 6

The TSLP binding protein according to embodiment 5, wherein CDR3 consists of the sequence X₁GlnX₂X₃X₄AspProX₅Thr, wherein X₁ represents Lys, Trp, Val or Met, X₂ represents Val, Leu or Ile, X₃ represents Gly or Ala, X₄ represents Glu, Phe, Asp or Ser, and X₅ represents Val or Thr.

### Embodiment 7

The TSLP binding protein according to embodiment 1 that comprises CDR1, CDR2, and CDR3 of SEQ ID NO: 9.

### Embodiment 8

The TSLP binding protein according to embodiment 7, wherein CDR1 consists of the sequence defined as SEQ ID NO: 1, CDR2 consists of the sequence defined as SEQ ID NO: 4, and CDR3 consists of the sequence defined as SEQ ID NO:7.

### Embodiment 9

The TSLP binding protein according to embodiment 7, wherein CDR1 consists of the sequence defined as SEQ ID NO: 1, CDR2 consists of the sequence defined as SEQ ID NO: 5 and CDR3 consists of the sequence defined as SEQ ID NO:7.

### Embodiment 10

The TSLP binding protein according to any preceding embodiment, wherein the TSLP binding protein binds to full length human TSLP with a dissociation constant (KD) of less than 2 nM.

### Embodiment 11

The TSLP binding protein as according to embodiment 1, wherein the TSLP binding protein competes for binding to full-length human TSLP with a single variable domain of SEQ ID NO:9.

### Embodiment 12

The TSLP binding protein according to embodiment 1, wherein the TSLP binding protein exhibits no significant binding to IL-7.

### Embodiment 13

The TSLP binding protein according to embodiment 1, wherein the TSLP binding protein is a single variable domain.

### Embodiment 14

The TSLP binding protein according to embodiment 13, wherein the single variable domain is a V_{K} single variable domain.

### Embodiment 15

The TSLP binding protein according to embodiment 14, wherein the V_{K} single variable domain has a C-terminus ending in RT.

### Embodiment 16

The TSLP binding protein according to embodiment 14, wherein the V_{K} single variable domain has a C-terminus that does not end in R.

### Embodiment 17

The TSLP binding protein according to embodiment 14, wherein the residue corresponding to residue 27 in SEQ ID NO:9 is Arg, the residue corresponding to residue 29 in SEQ ID NO: 9 is Ile, the residue corresponding to residue 89 in SEQ ID NO:9 is Val, and the residue corresponding to residue 96 in SEQ ID NO: 9 is Val.

### Embodiment 18

The TSLP binding protein according to embodiment 14, wherein the residue corresponding to residue 49 in SEQ ID NO:9 is Trp.

### Embodiment 19

The TSLP binding protein according to embodiment 14, wherein the residue corresponding to residue 36 in SEQ ID NO:9 is Tyr, the residue corresponding to residue 38 in SEQ ID NO:9 is Gln, the residue corresponding to residue 44 in SEQ ID NO:9 is Pro, the residue corresponding to residue 67 in SEQ ID NO:9 is Ser, the residue corresponding to residue 87 in SEQ ID NO:9 is Tyr, the residue corresponding to residue 98 in SEQ ID NO:9 is Phe, and the residue corresponding to residue 100 in SEQ ID NO:9 is Gln.

### Embodiment 20

The TSLP binding protein that consists of the amino acid sequence of SEQ ID NO. 9.

### Embodiment 21

An isolated nucleic acid encoding the TSLP binding protein as defined in embodiment 1.

### Embodiment 22

An isolated nucleic acid molecule according to embodiment 21, wherein the nucleic acid molecule is selected from the group consisting of: SEQ ID NO:10 and SEQ ID NO:11.

### Embodiment 23

A vector comprising the nucleic acid molecule as defined in embodiment 21 or embodiment 22.

### Embodiment 24

A host cell comprising a nucleic acid as defined in embodiment 21 or 22 or a vector as defined in embodiment 23.

### Embodiment 25

A method of producing a TSLP binding protein as defined in embodiment 1, the method comprising the steps of: maintaining a host cell as defined in embodiment 24 under conditions suitable for expression of said nucleic acid or vector, whereby a TSLP binding protein is produced.

### Embodiment 26

A method of treating a disease associated with TSLP signalling in a patient in need thereof comprising administering to the patient a therapeutically effective amount of the TSLP binding protein as defined in embodiment 1

### Embodiment 27

The method of treatment according to embodiment 26, wherein the patient is human.

### Embodiment 28

The method of treatment according to embodiment 27, wherein the disease associated with TSLP signalling is selected from the group consisting of: asthma, idiopathic pulmonary fibrosis, atopic dermatitis, allergic conjunctivitis, allergic rhinitis, Netherton syndrome, eosinophilic esophagitis (EoE), food allergy, allergic diarrhoea, eosinophilic gastroenteritis, allergic bronchopulmonary aspergillosis (ABPA), allergic fungal sinusitis, cancer, rheumatoid arthritis, COPD, systemic sclerosis, keloids, ulcerative colitis, chronic rhinosinusitis (CRS), nasal polyposis, chronic eosinophilic pneumonia, eosinophilic bronchitis, coeliac disease, Churg-Strauss syndrome, eosinophilic myalgia syndrome, hypereosinophilic syndrome, eosinophilic granulomatosis with polyangiitis, and inflammatory bowel disease.

### Embodiment 29

The method of treatment according to embodiment 28, wherein the disease associated with TSLP signalling is selected from the group consisting of: asthma, idiopathic pulmonary fibrosis, atopic dermatitis, allergic conjunctivitis, allergic rhinitis, Netherton syndrome, eosinophilic esophagitis (EoE), food allergy, allergic diarrhoea, eosinophilic gastroenteritis, allergic bronchopulmonary aspergillosis (ABPA), allergic fungal sinusitis, cancer, rheumatoid arthritis, COPD, systemic sclerosis, keloids, ulcerative colitis, chronic rhinosinusitis (CRS) and nasal polyposis.

### Embodiment 30

The method of treatment according to embodiment 29, wherein the disease associated with TSLP signalling is asthma.

### Embodiment 31

A pharmaceutical composition comprising a TSLP binding protein as defined in embodiment 1, and optionally, at least one pharmaceutically acceptable ingredient selected from the group consisting of: an excipient and a carrier.

### Embodiment 32

A kit comprising a TSLP binding protein as defined in embodiment 1 and a device for inhaling said TSLP binding protein.

### Embodiment 33

A TSLP binding protein that binds an epitope comprising the following residues of full-length human TSLP: Tyr15, Lys31, Ser32, Thr33, Phe35, Asn36, Asn37, Ser40, Cys41, Ser42, Ser114, Gln115, Gln117, Gly118, Arg121, Arg122, Arg125, Pro126, Leu128, and Lys 129.

### Embodiment 34

A TSLP binding protein according to embodiment 33, wherein the epitope further comprises the following residues: Ser20, Ile24, Glu34, Thr38, Val39, Asn43, His46, Asn124, and Leu127.

### Embodiment 35

The TSLP binding protein according to embodiment 33, wherein the TSLP binding protein is an antibody.

### Embodiment 36

The TSLP binding protein according to embodiment 33, wherein the TSLP binding domain is a single variable domain.

### Embodiment 37

The TSLP binding protein according to embodiment 36, wherein the TSLP binding domain is a V_{K} domain.

### Embodiment 38

The TSLP binding protein according to embodiment 33, wherein the TSLP binding domain exhibits no significant binding to IL-7.

### EXAMPLES

### Example 1 Identification of Naive dAbs that Bind TSLP

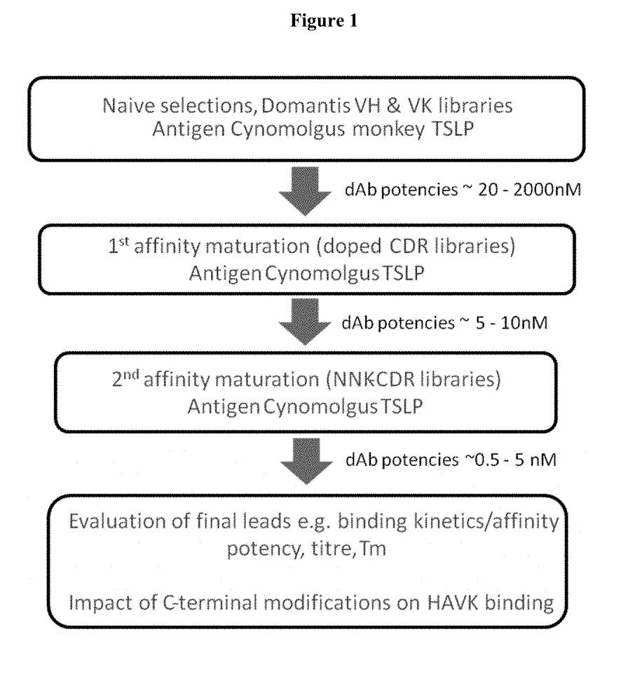

Domain antibodies specific for TSLP were identified using standard phage display techniques. Domantis naïve phage libraries displaying antibody single variable domains were used for panning against biotinylated recombinant Cynomolgus TSLP. dAbs which bound to TSLP were identified by dAb ELISA. TSLP-specific dAbs were further characterised by Surface Plasmon Resonance assay and/or in the TSLP Receptor Binding Assay (RBA). The identity of positive hits was determined by sequencing, and these hits are termed ‘naive dAbs’. In these initial naive selections, the aim was to identify dAbs which had a potency (IC50 measured by RBA or cell assay) in the range of 20-2000 nM. A flow chart illustrating this and subsequent steps involved in generating dAb DOM30h-440-81/86 and dAb Dom30h-440-87/93, and other high affinity dAbs with desirable properties disclosed herein, is given in FIG. 1.

dAb ELISA Screening

Human and cynomolgus TSLP-specific dAbs were identified by ELISA. Briefly, 96-well Maxisorp™ immuno plates (Nunc, Denmark) precoated with neutravidin were coated with biotinylated cynomolgus TSLP or biotinylated human TSLP overnight at 4° C. Wells were washed with PBST and then blocked with 2% Marvel in PBS (2% MPBS). dAb supernatant were added at a 1:1 mixture in 2% MPBS. Bound dAb was detected using a monoclonal anti-FLAG M2-peroxidase conjugated antibody, (Sigma-Aldrich, UK). For detection of the peroxidase conjugated antibody, a colourimetric substrate was used, (SureBlue 1-component TMB Microwell Peroxidase solution) and optical density (OD) measured at 450 nm. Positive binders for cynomolgus TSLP were identified where OD450 was 2× assay background.

Surface Plasmon Resonance (SPR) Assay (Primary Amine Coupling of TSLP)

Using a BIACORE™ T200, dAbs were assessed for binding kinetics and affinity for binding to human TSLP, cynomolgus TSLP and human IL-7 cytokines. Human TSLP, cynomolgus TSLP and Human IL-7 were immobilized on a CM4 chip by primary amine coupling. Test dAbs were passed over the immobilized cytokines at 160 nM, 40 nM, 10 nM, 2.5 nM, 0.63 nM and 0.156 nM in HBS-EP buffer and binding curves were recorded. This was run in duplicate at 25° C. within the same BIAcore run. The curves were double-referenced using a buffer injection curve and then fitted to the 1:1 binding model inherent to the Biacore T200 Evaluation software.

TSLP Receptor Binding Assay (RBA)

To identify dAbs with TSLP neutralisation activity, soluble dAbs were tested for their ability to block TSLP binding to its receptor complex. The extracellular domains of the human TSLPR and IL-7R (R&D Systems) were coated onto ELISA plates to self associate and form the TSLP receptor heterodimer. dAbs were either tested at a single concentration, or diluted in a concentration range (for example 3.8 pM-1 μM) and pre-incubated for one hour with either human or cynomolgus monkey TSLP at a predetermined concentration (e.g., 1.5 ng/ml). The dAb-TSLP complexes were then added to microwell plates for 2 hours and the amount of bound TSLP was quantified using either a biotinylated TSLP detection antibody and steptavidin:HRP (absorbance measured at 450 nM using a SpectraMax plate reader) or with a ruthenylated TSLP detection antibody (electrochemiluminescence measured using an MSD Sector Imager). Data were plotted using a 4 parameter logistic fit model to obtain potency values.

Assay to Determine Concentration of dAbs in Supernatants

dAbs expressed with a C-terminal FLAG tag were quantified in supernatants by detection of the FLAG epitope. The assay relies on an HTRF signal between a Cy3b-labelled FLAG peptide and a Terbium-labelled anti-FLAG antibody. dAbs expressed with a FLAG-tag are able to compete for this interaction and reduce the HTRF signal.

A control dAb of known concentration with a C-terminal FLAG-tag was serially diluted in 2xYT culture medium and then diluted further 1:10 in PBS to reduce the final concentration of the culture medium. 7 μl of each sample was added to wells of a 384 well white LV assay plate (Greiner). This served as a dAb standard curve.

Test dAbs (supernatants) of unknown concentration were diluted in 2xYT medium (neat, 1:2, 1:4, 1:8) then diluted further 1:10 in PBS to reduce the final concentration of the culture medium. 7 μl/well of each sample was added to the assay plate.

Fluorescently labelled FLAG peptide (H-Asp-Tyr-Lys-Asp-Asp-Asp-Asp-Lys-Gly-Gly-Cys(Cy3B)—OH) (Cambridge Research Biochemicals) was prepared at a final concentration of 1 μM in a mixture with anti-Flag M2 Terbium-labelled antibody (Cisbio catalog number 61FG2TLB) (1/2000 dilution) in 0.2 mM BSA and 1 mM CHAPS buffer.

7 μl of the FLAG peptide/anti-FLAG mAb mixture is added to each well and the plate is spun at 1000 rpm for 1 minute to collect the solution at the bottom of the well. The plate is incubated at room temperature for 10 minutes in the dark and then read fluorescence was read using an Envision plate reader (Perkin Elmer). HTRF emissions were measured at two different wavelengths, 615 nm (donor) and 665 nm (acceptor) to reduce well to well variations (ratiometric measurement). The concentration of dAbs in supernatant samples was determined from the standard curve of serially diluted control dAb.

Assay to Determine Concentration of Purified dAbs

The concentration of purified dAbs in buffer was determined spectrophotometrically by measurement of the absorbance of UV light at 280 nM using a Nanodrop 1000 instrument (Thermo Scientific).

### Example 2: Affinity Maturation to Increase the Affinity of dAbs for TSLP

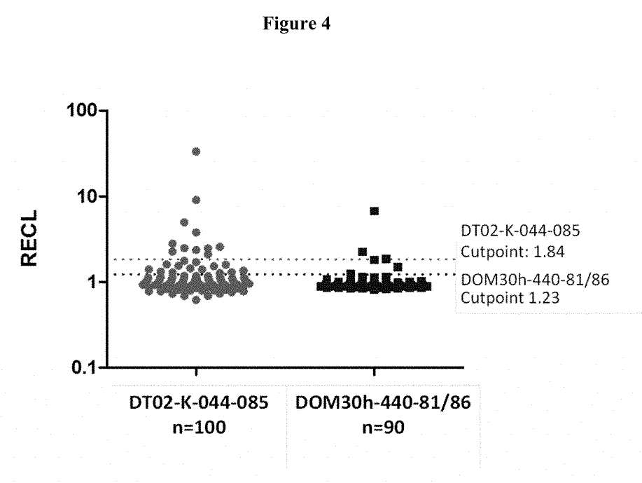

Affinity maturation was performed on the DOM30h-440 naive dAb using degenerative mutagenesis to re-diversify the CDR regions. CDR diversification was carried out with doped libraries which were constructed using a single degenerate oligonucleotide primer designed to cover all mutations within each CDR. The amino acids to be diversified were specified using degenerate codons allowing multiple amino acids to be encoded at a single position. Re-diversified dAbs were subject to 5 rounds of selection against decreasing amounts of biotinylated cynomolgus TSLP antigen (100 nM, 50 nM, 10 nM, 1 nM). Examples of dAbs identified are shown in FIG. 4.

TABLE 4
Identification of anti-TSLP dAbs by screening in TSLP receptor binding assay
(RBA)

| | Sequence | | | | | | | | |
|---|---|---|---|---|---|---|---|---|---|
| | identity | | | | | | | | |
| | with | | | | | | | | |
| | DOM30h- | Cynomolgus TSLP | | | | Human TSLP | | | |
| | 440- | Geometric | | | | Geometric | | | |
| Clone | 81/86 | Mean | +/−SD | | KD | Mean | +/−SD | | KD |
| name | (%) | IC50 (nM) | (nM) | N | (nM) | IC50 (nM) | (nM) | n | (nM) |
| DOM30h- | 94.39 | 2050 | 586-7171 | 7 | 622.6 | 572 | 146-2240 | 7 | 86.5 |
| 440 | | | | | | | | | |
| DOM30h- | 94.39 | 159 | N/A | 1 | 27.5 | 19 | N/A | 1 | 18.4 |
| 440-1 | | | | | | | | | |
| DOM30h- | 94.39 | 1666 | N/A | 1 | ND | 80 | N/A | 1 | ND |
| 440-2 | | | | | | | | | |
| DOM30h- | 95.33 | 542 | N/A | 1 | 73.7 | 106 | N/A | 1 | 41.1 |
| 440-3 | | | | | | | | | |
| DOM30h- | 95.33 | 483 | 330-706 | 2 | ND | 132 | 21-842 | 2 | ND |
| 440-30 | | | | | | | | | |
| DOM30h- | 95.33 | 605 | 568-644 | 2 | ND | 289 | 137-610 | 2 | ND |
| 440-31 | | | | | | | | | |
| DOM30h- | 94.39 | 1015 | 716-1438 | 2 | ND | 89 | 50-160 | 2 | ND |
| 440-32 | | | | | | | | | |
| DOM30h- | 95.33 | 309 | 224-427 | 2 | 43.4 | 45 | 35-56 | 2 | 33.8 |
| 440-33 | | | | | | | | | |
| DOM30h- | 95.33 | 59 | 38-91 | 8 | 28.5 | 8 | 3.1-19 | 8 | 17.4 |
| 440-34 | | | | | | | | | |
| DOM30h- | 98.13 | 65 | 29-142 | 6 | 26.1 | 4 | 2.6-7.5 | 4 | 16.3 |
| 440-35 | | | | | | | | | |
| DOM30h- | 94.39 | 466 | 312-697 | 3 | 52.5 | 83 | 70-98 | 2 | 36.4 |
| 440-37 | | | | | | | | | |
| DOM30h- | 93.46 | 5825 | 3406-9964 | 2 | ND | >16982 | N/A | 1 | ND |
| 440-38 | | | | | | | | | |
| DOM30h- | 95.33 | 62 | 24-163 | 4 | 45.6 | 8 | 2-27 | 3 | 61.6 |
| 440-39 | | | | | | | | | |
| DOM30h- | 95.33 | 975 | N/A | 1 | 394 | NT | | | 89.9 |
| 440-4 | | | | | | | | | |
| DOM30h- | 95.33 | 549 | 185-1630 | 4 | 307.6 | 32 | 11-94 | 3 | 119.6 |
| 440-40 | | | | | | | | | |
| DOM30h- | 95.33 | 142 | 61-329 | 4 | 283.6 | 68 | 36-129 | 2 | 280.6 |
| 440-41 | | | | | | | | | |
| DOM30h- | 95.33 | 104 | 17-635 | 4 | 79.2 | 57 | N/A | 1 | 70.7 |
| 440-42 | | | | | | | | | |
| DOM30h- | 93.46 | 6690 | N/A | 1 | ND | 1308 | 841-2034 | 2 | ND |
| 440-43 | | | | | | | | | |
| DOM30h- | 95.33 | 2090 | N/A | 1 | ND | 673 | N/A | 1 | ND |
| 440-6 | | | | | | | | | |
| DOM30h- | 95.33 | 97 | N/A | 1 | 78.1 | NT | | | 11.8 |
| 440-7 | | | | | | | | | |
| DOM30h- | 95.33 | 796 | N/A | 1 | 279.8 | 23 | N/A | 1 | 74.6 |
| 440-9 | | | | | | | | | |

Further affinity maturation was performed on the DOM30h-440-35 dAb using the phagemid system, enabling a 1:1 interaction with the target antigen (biotinylated TSLP) during selections and allowing the selection of improved dAb clones based on intrinsic affinity. Second round affinity maturation was performed using two mutagenic approaches, NNK walking and error prone mutagenesis. NNK walking lead to re-diversification of the CDR regions with NNK libraries which were constructed using a single degenerate oligonucleotide primer designed to cover all mutations for the targeted residues. Error prone mutagenesis subjected the whole dAb sequence to diversification at a medium mutation rate of 4.5-9 amino acid changes per dAb, this included both the framework and CDR regions. Both the NNK phagemid libraries and the error prone libraries were subjected (individually or as a pool with other libraries) to 4 rounds of selection against 10 nM, 1 nM, 1 nM, 0.1 nM (rounds 1, 2, 3, 4 respectively) biotinylated cynomolgus TSLP. dAbs were sequenced and screened by TSLP receptor binding assay (RBA) (methodology in Example 1) and/or tested in a cell based assay (methodology below). The affinity of purified dAbs was determined by surface plasmon resonance (SPR) (methodology below).

Cell Assay (Inhibition of TSLP-Induced pSTAT5 in SW756 Cells)

Affinity matured dAbs were assessed to determine the potency at inhibiting TSLP stimulated phosphorylation of Signal Transduction and Activator of Transcription 5 in the vaginal carcinoma cell line SW756(ATCC). These cells express endogenous TSLP receptors as determined by mRNA analysis and have been shown to respond to TSLP as demonstrated by STATS phosphorylation. In brief, SW756 cells were seeded into 96-well plates at a density of 25,000 cells/well and incubated overnight at 37° C. in 5% CO₂ to allow adherence. Human or cynomolgus TSLP, at an EC₇₅ concentration of 1 ng/ml, was pre-incubated with dAbs at a concentration range of 0.05-1000 nM for one hour. The TSLP/dAb complex was added to the cells and incubated for 30 minutes at 37° C. in 5% CO₂, followed by cell lysis. Lysates were analysed by MesoScale Discovery (MSD) to quantify pSTAT5 as according to the manufacturer's protocol (K15163D-3) using an MSD Sector Imager 6000. Data were plotted using a 4 parameter logistic fit model to obtain potency values.

Surface Plasmon Resonance (SPR) Assay (Biotinylated TSLP)

dAbs were assessed for binding kinetics and affinity for binding to biotinylated human TSLP and biotinylated cynomolgus TSLP. This was measured using a BIACORE™ 4000. biotinylated human TSLP and biotinylated cynomolgus TSLP were immobilized on a SA chip by SA-Biotin coupling. Test dAbs were passed over the immobilized cytokines at 1000 nM, 100 nM, 10 nM and 0 nM in HBS-EP buffer and binding curves were recorded. This was run at 25° C. within the same BIAcore run. The curves were double referenced using a buffer injection curve and then fitted to the 1 to 1 binding model inherent to the BIACORE™ 4000 Evaluation software.

The aim of affinity maturation screening was to identify dAbs which had a potency (IC50 measured by RBA or cell assay) less than 5 nM and retained good cross-reactivity with cynomolgus TSLP (preferably less than 5-fold difference in IC50 between human and cynomolgus TSLP). Table 5 lists clones that had a potency (IC50) of less than 5 nM in the RBA or cell assay.

TABLE 5
Characterisation of dAbs

| | % | | | | | | | | |
|---|---|---|---|---|---|---|---|---|---|
| | Sequence | | | | | | | | |
| | identity | | | pSTAT5 assay | | | | | |
| | with | ELISA RBA | | in SW756 cells | | | | | |
| | DOM30h- | Geometric | | Geometric | | | | | |
| | 440- | Mean IC50 | | Mean IC50 | | Affinity (KD) | | Small- | |
| | 81/86 | (nM) | | (+/−SD) (nM) | | (nM) | | scale | |
| Clone | (excluding | Human | Cynomolgus | Human | Cynomolgus | Human | Cynomolgus | Yield | Tm |
| Number | CDRs) | TSLP | TSLP | TSLP | TSLP | TSLP | TSLP | (mg/L) | ° C. |
| DOM30h- | 97 (98) | 2.00 | 1.30 | 1.92 | 7.28 | 3.05 | 5.79 | 48 | 52.2 |
| 440- | | (0.65-6.11) | (1.20-1.40) | (1.41-2.61) | (3.11-17.01) | | | | |
| 53 | | | | | | | | | |
| DOM30h- | (98) | 2.15 | 0.67 | 1.52 | 2.45 | 3.82 | 6.24 | 7 | 40.4 |
| 440- | | (1.31-3.50) | (0.46-0.97) | (0.83-2.75) | (1.22-4.92) | | | | |
| 54 | | | | | | | | | |
| DOM30h- | 99 (98) | 0.87 | 0.55 | 1.10 | 3.12 | 2.78 | 1.61 | 49 | 54.8 |
| 440- | | (0.67-1.14) | (0.40-0.76) | (0.63-1.80) | (1.34-7.30) | | | | |
| 55 | | | | | | | | | |
| DOM30h- | 97 (97) | 0.56 | 0.55 | 0.99 | 4.74 | 2.47 | 2.97 | 95 | 43.2 |
| 440- | | (0.48-0.65) | (0.28-1.07) | (0.88-1.13) | (2.31-9.74) | | | | |
| 56 | | | | | | | | | |
| DOM30h- | 96 (97) | 0.34 | 0.36 | 0.56 | 1.60 | 1.50¹ | 1.92² | 48 | 45.2 |
| 440- | | (0.26-0.44) | (0.35-0.37) | (0.32-0.98) | (0.85-3.02) | | | | |
| 57 | | | | | | | | | |
| DOM30h- | (98) | 1.41 | 0.21 | 1.91 | 3.43 | 2.65 | 3.42 | 46 | 49.2 |
| 440- | | (0.88-2.28) | (0.11-0.40) | (1.37-2.65) | (1.7-6.90) | | | | |
| 58 | | | | | | | | | |
| DOM30h- | 97 (98) | 0.32 | 2.65 | 0.70 | 11.24 | 0.14 | 7.81 | 51 | 54.2 |
| 440- | | (0.16-0.63) | (1.52-4.60) | (0.40-1.20) | (5.95-21.23) | | | | |
| 60 | | | | | | | | | |
| DOM30h- | 97 (98) | 1.55 | 0.83 | 2.21 | 2.40 | 4.22 | 5.98 | 43 | 47.6 |
| 440- | | (1.42-1.70) | (0.64-1.08) | (1.90-2.61) | (0.75-7.79) | | | | |
| 63 | | | | | | | | | |
| DOM30h- | 98 (98) | 0.95 | 0.87 | 3.26 | 3.36 | 6.17 | 4.29 | 87 | 50.6 |
| 440- | | (0.54-1.65) | (0.69-1.10) | (1.38-7.70) | (1.74-6.48) | | | | |
| 64 | | | | | | | | | |
| DOM30h- | 98 (98) | 0.69 | 0.29 | 0.78 | 3.8 | 3.84 | 3.11 | 29 | 49.8 |
| 440- | | (0.45-1.05) | (0.17-0.48) | (0.37-1.66) | (0.70-20.21) | | | | |
| 65 | | | | | | | | | |

¹Value excluding 1000 nM concentration (3.48 with all concentrations)
²Value excluding 1000 nM concentration (4.95 with all concentrations)

### Example 3: Identification of “Consensus” CDRL3 for Clones with IC50<5 nM

Table 6 provides the sequences of CDR3 (according to the Kabat numbering convention) for each of the clones listed in Table 5.

TABLE 6
CDR sequences of dAbs

| Clone number | Sequence Identifier | CDRL3 |
|---|---|---|
| DOM30h-440-53 | SEQ ID NO: 23 | LQVGEDPVT |
| | | (SEQ ID NO: 15) |
| DOM30h-440-54 | SEQ ID NO: 24 | WQLAFDPTT |
| | | (SEQ ID NO: 16) |
| DOM30h-440-55 | SEQ ID NO: 25 | VQIGEDPVT |
| | | (SEQ ID NO: 7) |
| DOM30h-440-56 | SEQ ID NO: 26 | MQIGEDPVT |
| | | (SEQ ID NO: 17) |
| DOM30h-440-57 | SEQ ID NO: 27 | MQIGDDPVT |
| | | (SEQ ID NO: 18) |
| DOM30h-440-58 | SEQ ID NO: 28 | LQIADDPVT |
| | | (SEQ ID NO: 19) |
| DOM30h-440-60 | SEQ ID NO: 29 | IQFGEDPVT |
| | | (SEQ ID NO: 20) |
| DOM30h-440-63 | SEQ ID NO: 30 | MQIGSDPVT |
| | | (SEQ ID NO: 21) |
| DOM30h-440-64 | SEQ ID NO: 31 | LQIGEDPVT |
| | | (SEQ ID NO: 22) |
| DOM30h-440-65 | SEQ ID NO: 32 | MQIGEDPVT |
| | | (SEQ ID NO: 17) |

There is variation in the sequence of CDRL3 across the clones. It is possible to identify a consensus sequence for CDRL3 that includes the sequence CDRL3 from all clones having an IC50 of less than or equal to 5 nM in the receptor binding assay using human TSLP and/or in the cell assay using human TSLP: X₁GlnX₂X₃X₄AspProX₅Thr, wherein X₁ represents Lys, Trp, Val, Met or Ile, X₂ represents Val, Leu, Ile or Phe, X₃ represents Gly or Ala, X₄ represents Glu, Phe, Asp or Ser, and X₅ represents Val or Thr.

If, in addition, it is required that there is less than or equal to 5 fold difference in IC50 in the cell assay using human and cynomolgus TSLP, the consensus sequence becomes: X₁GlnX₂X₃X₄AspProX₅Thr, wherein X₁ represents Lys, Trp, Val or Met, X₂ represents Val, Leu or Ile, X₃ represents Gly or Ala, X₄ represents Glu, Phe, Asp or Ser, and X₅ represents Val or Thr.

### Example 4: C-Terminal Modification of Affinity Matured dAbs Reduces Binding to Pre-Existing Human Anti-V_{K} (HAVK) Antibodies

DOM30h-440-55 and DOM30h-440-57 are examples of dAbs identified after affinity maturation which have improved potency against human and cynomolgus monkey TSLP. In the case of DOM30h-440-55 this dAb also was shown to have a higher thermal stability (Tm). C-terminal modifications and other framework mutations to reduce binding to pre-existing HAVK antibodies were made to these dAbs as detailed in Table 7. Dom30h-440-87, Dom30h-440-88, Dom30h-440-90 and Dom30h-440-91 were tested in an assay to determine the impact of the C-terminal modification on binding to pre-existing HAVK antibodies.

Assay for Binding to Pre-Existing HAVK Antibodies

A V_{K} dAb that does not have a modified C-terminus (DT02-K-044-085 dAb-amino acid sequence published in WO2013014208 as SEQ ID NO:105) was used to develop an assay to test whether C-terminal modifications to the Vk dAb framework of anti-TSLP dAbs could reduce binding to pre-existing human anti-V_{K} (HAVK) antibodies which bind the Vk framework (confirmation assay). Serum samples from 10 known HAVK positive human donors were used in the assay.

In a microtitre assay plate, the sample containing HAVK positive human serum sample and test material (such as DT02-K-044-085 dAb control or modified dAbs) is pre-incubated for 1 hour at room temperature, then added to a homogeneous mixture of biotinylated DT02-K 044-085 and ruthenylated (“Sulfo-Tag”™) DT02-K-044-085 dAb in assay diluent (1% casein in PBS) such that the final concentrations are 5% HAVK positive human serum, 10 μg/mL test material (such as DT02-K-044-085 dAb or modified dAbs), 0.2 μg/mL biotinylated DT02-K-044-085 and 0.1 μg/mL ruthenylated (“Sulfo-Tag”™) DT02-K-044-085 dAb. The mixture is incubated for 1 hour at RT and then the assay samples are transferred to an MSD™ streptavidin plate (previously blocked with 150 μL/well casein in PBS (1%) at RT for 1-2 hours and the blocker removed without washing). The MSD™ plate is incubated for 1 hour in the dark at RT then washed 3 times, 150 μL/well read buffer is added and the plate is read on the MSD Sector Imager.

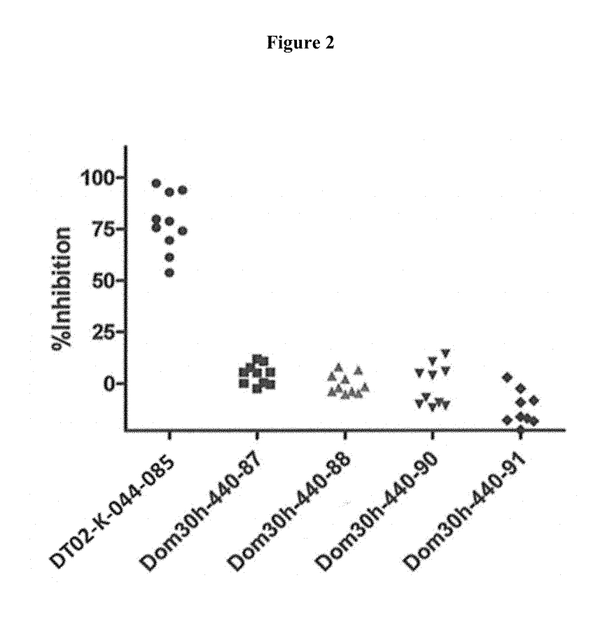

The luminescence signal in the assay is generated when the biotinylated and ruthenylated molecules of DT02-K-044-085 dAb are cross-linked by antibodies present in the sample. Free, unlabelled DT02-K-044-085 dAb competes for HAVK binding in this assay resulting in reduced signal intensity (high % signal inhibition). This assay was used to determine whether modified versions of Vk anti-TSLP dAbs could compete with DT02-K-044-085 dAb for ADA binding. Results are shown in FIG. 2 as the % inhibition of signal. The lower the % inhibition of signal the less the modified dAb was able to bind to HAVK antibodies. Using the confirmation assay, it was determined that dAbs with either −R or + T modifications at the C-terminus had reduced binding to pre-existing HAVK antibodies compared with DT02-K-044-085 dAb.

TABLE 7
Generation of dAbs with modifications

| | Framework | | | |
|---|---|---|---|---|
| | mutation | | Non codon | Codon |
| | compared | C-terminal | optimised | optimised |
| Parent dAb | with parent | modification | name | name |
| DOM-30h-440-55 | None | −R | DOM30h-440-81 | DOM30h-440-86 |
| DOM-30h-440-55 | K45E | −R | DOM30h-440-93 | DOM30h-440-87 |
| DOM-30h-440-57 | M89V | −R | DOM30h-440-92 | DOM30h-440-88 |
| DOM-30h-440-55 | None | +T | DOM30h-440-94 | DOM30h-440-89 |
| DOM-30h-440-55 | K45E | +T | DOM30h-440-95 | DOM30h-440-90 |
| DOM-30h-440-57 | M89V | +T | DOM30h-440-96 | DOM30h-440-91 |

Sequence identifiers for the amino acid sequences of the modified dAbs are listed below:

### SEQ ID NO: 9: Dom30h-440-81/86

### SEQ ID NO: 12: Dom30h-440-87/93

### SEQ ID NO: 33: Dom30h-440-88/92

### SEQ ID NO: 34: Dom30h-440-89/94

### SEQ ID NO: 35: Dom30h-440-90/95

### SEQ ID NO: 36: Dom30h-440-91/96

### Example 5: Further Characterisation of DOM30h-440-81/86

Both [−R] and [+T] C-terminal modifications were shown to be equally effective in reducing binding of dAbs to pre-existing HAVK antibodies. A theoretical risk of clipping of the [+T] C-terminal residue in vivo to yield the original C-terminus was considered. Therefore DOM30h-440-81/86 (−R) and DOM30h-440-87/93 (−R) were selected from the second round affinity maturation based on a combination of high potency, cross-reactivity with cynomolgus monkey TSLP and acceptable yield at small scale. Of the two DOM30h-440-81/86 was preferred due to higher thermal stability (Tm).

The affinity of DOM30h-440-81/86 for human and cynomolgus monkey TSLP was determined by SPR with biotinylated TSLP as described previously. Example data are shown in Table 8.

TABLE 8
Affinity of DOM30h-440-81/86 for recombinant human and cynomolgus
monkey TSLP (Mean data derived from two experiments)

| Ligand | ka (1/Ms) | kd (1/s) | KD (M) | KD (nM) |
|---|---|---|---|---|
| Human | 6.19 × 10⁵ | 5.08 × 10⁻⁴ | 8.67 × 10⁻¹⁰ | 0.87 |
| TSLP | | | | |
| Cynomolgus | 1.62 × 10⁶ | 7.74 × 10⁻⁴ | 5.11 × 10⁻¹⁰ | 0.51 |
| TSLP | | | | |
| Human IL-7 | No | No | No | No |
| | significant | significant | significant | significant |
| | Binding | Binding | Binding | Binding |

The potency for inhibition of TSLP in the RBA and potency for inhibition of TSLP-induced pSTAT5 in SW756 cells was determined as described previously (Table 9). The potency of DOM30h-440-81/86 for inhibition of human TSLP-induced TARC (CCL17) in human whole blood was also determined. Example data are shown in Table 9.

Inhibition of TSLP-Induced TARC (CCL17) in Human Whole Blood

Blood from healthy volunteer donors (with appropriate consent compliant with the UK Human Tissue Act) in sodium heparin (1000 IU/100 ml) was obtained from the GSK Stevenage Blood Donation Unit. dAbs were diluted at a concentration range (for example from 0.04 nM-100 nM) followed by pre-incubation with an EC₇₅ concentration of recombinant human TSLP (1 ng/ml) for one hour at room temperature. Blood from each donor was added to the TSLP:dAb complex and incubated for a further 48 hours at 37° C. and 5% CO₂. Plasma was then harvested and frozen at −80° C. for analysis of TARC levels by MSD as described in the manufacturer's protocol (K151BGC-4) using an MSD Sector Imager. Data were plotted using a 4 parameter logistic fit model to obtain potency values.

TABLE 9
Potency of DOM30h-440-81/86 for inhibition of human
and cynomolgus monkey TSLP expressed from E. Coli

| | DOM30h-440-81/86 Geometric Mean IC50 (+/−SD) nM | | |
|---|---|---|---|
| | Inhibition of | Inhibition of | Inhibition of |
| | binding of TSLP | TSLP-induced | TSLP-induced |
| | to TSLP receptor | pSTAT5 in | TARC in |
| | complex (RBA) | SW756 cells | whole blood |
| | (n = 2) | (n = 5) | (n = 7) |
| Human TSLP | 1.94 | 1.92 | 2.4 |
| from E. coli | (1.32-2.85) | (0.85-4.36) | (0.4-14.4) |
| Cynomolgus | 2.05 | 4 | Not Done |
| TSLP | (1.13-3.72) | (2.9-4.4) | |
| from E. Coli | | | |

### Example 6: Potency of DOM30h-440-81/86 for Inhibition of Glycosylated Recombinant Human TSLP Expressed from HEK Cells

Using the cell assay (inhibition of TSLP-induced pSTAT5 in SW756 cells), or the whole blood assay (inhibition of TSLP-induced TARC (CCL17) in human whole blood), the potency of DOM30h-440-81/86 to inhibit human TSLP expressed from human embryonic kidney (HEK) cells was determined.

TABLE 10

DOM30h-440-81/86 Geometric Mean
IC50 (+/−SD) nM

| | Inhibition of | Inhibition of |
|---|---|---|
| | TSLP-induced | TSLP-induced |
| | pSTAT5 in SW756 | TARC in whole |
| | cells (n = 3) | blood (n = 6) |
| Human | 0.55 | 3.10 |
| TSLP from | (0.43-0.70) | (1.6-6.2) |
| HEK cells | | |

### Example 7 DOM30h-440-81/86 Inhibits Native Human TSLP

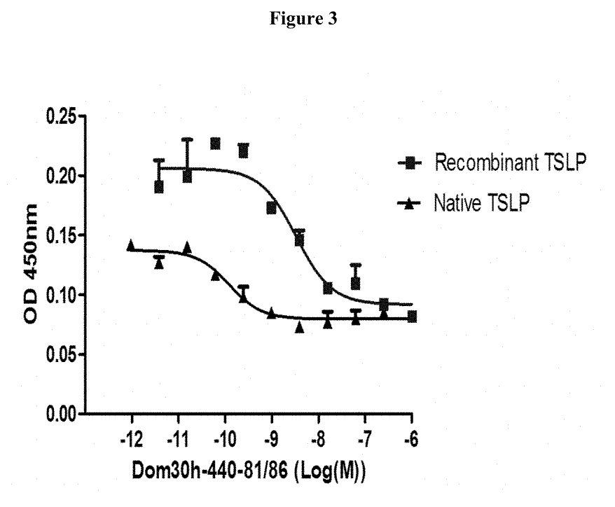

Supernatant from human lung fibroblasts that had been stimulated with inflammatory cytokines (10 ng/ml IL-1β and 10 ng/ml TNFα) for 48 hours was used a source of native human TSLP. Native human TSLP was used in the RBA assay to determine potency. In these experiments native TSLP gave a lower assay signal, but it was determined that DOM30h-440-81/86 inhibited native TSLP in a dose-dependent manner. Example data are shown in FIG. 3.

Similarly, a supernatant from human lung fibroblasts that had been stimulated with inflammatory cytokines (1 ng/ml TNFα and 10 ng/ml IL-4 for 48 h) was used a source of native TSLP (approximately 2 ng/ml). Using the cell assay (inhibition of TSLP-induced pSTAT5 in SW756 cells) DOM30h-440-81/86 inhibited native TSLP-induced STATS phosphorylation with a Geometric Mean IC50 (+/−SD) of 0.86 nM (0.51-1.45 nM).

### Example 8 DOM30h-440-81/86 Inhibits IL-5 Production from Human PBMC Stimulated with a Mixture of TSLP, IL-33 and IL-25

Human peripheral blood mononuclear cells (PBMC) were pre-incubated for 1 hour with DOM30h-440-81/86 before stimulating with 10 ng/ml each of human TSLP, human IL-25 and human IL-33 (R&D Systems). Cells were incubated for 96 hours at 37° C. in 5% CO₂ and the supernatants were harvested. IL-5 was measured using a bead based assay (Luminex). DOM30h-440-81/86 inhibited IL-5 production from human PBMC stimulated with a mixture of TSLP, IL-33 and IL-25 with a Geometric Mean IC50 (+/−SD) 129 nM+/−91 nM (mean 6 donors).

### Example 9: DOM30h-440-81/86 does not Bind to the Short Isoform of TSLP

Binding of DOM30h-440-81/86 to short form TSLP (sfTSLP—a synthetic biotinylated 63 amino acid peptide comprising residues 69-131 of mature full length TSLP) or full length TSLP:his was determined using a ForteBio Octet label-free interaction analysis instrument. A rabbit anti-TSLP polyclonal antibody (pAb) (Abeam catalog number ab47943) was used as a positive control for binding to both forms of TSLP. Streptavidin and anti-histidine (his) sensor tips were pre-incubated in IgG-free PBS buffer. Separately, biotinylated sfTSLP and full length TSLP:his were diluted to 10 μg/mL in IgG-free PBS buffer. The streptavidin sensor tips were then dipped into the biotinylated sfTSLP, and the anti-his sensor tips were dipped into the full length TSLP:his. Blank sensors without biotinylated sf TSLP and full length TSLP:his were also prepared by soaking in IgG-free PBS buffer. Next, sensors were dipped into solutions of DOM30h-440-81/86 or the polyclonal antibody at concentrations of 500 or 1000 nM and the binding response was measured. Buffer was also used as a blank control. The binding threshold was set at 0.1 response units and the study was performed at 25° C.

Under the conditions tested, the polyclonal antibody (ab47943) bound to both full length TSLP and sfTSLP. DOM30h-440-81/86 bound to full length TSLP protein but did not bind to sfTSLP. Some non-specific binding of the pAb to blank sensors was observed, but this was only when the much higher concentration of 1000 nM was used.

TABLE 11

| | | sfTSLP | | Full length TSLP | |
|---|---|---|---|---|---|
| Sample | | Response units | | Response units | |
| ID | Conc (nM) | sf TSLP | Blank | TSLP | Blank |
| TSLP dAb | 1000 | 0.04 | 0.02 | 0.32 | 0.02 |
| pAb 47943 | 1000 | 5.90 | 0.15 | 5.16 | 0.60 |
| Buffer | 0 | −0.01 | −0.01 | −0.12 | 0.01 |
| TSLP dAb | 500 | 0.07 | 0.00 | 0.25 | 0.04 |
| pAb 47943 | 500 | 4.53 | 0.02 | 3.69 | 0.01 |
| Buffer | 0 | 0.07 | 0.00 | −0.13 | 0.00 |

### Example 10 Anti-TSLP dAb Developability Assessment

An E. coli cell-line producing Dom30h440-81/86 showed superior titre and process robustness e.g. a 50 L fermentation process demonstrated titres of >2 g/L (n=5). Robustness was demonstrated in terms of growth, process control and titres. Plasmid stability was assessed at the 50 L scale, showing >95% yield of product expressing plasmid. A total process recovery of approximately 70% was achieved with desired product quality. A process has been developed for the manufacture and purification of Dom30h440-81/86 at a 150 L scale. FIG. 5 shows the fermentation process and FIG. 6 shows the downstream purification process. This process has been put into practice and a total product recovery of 64.5% was achieved. The product demonstrated binding in Biacore (using the methodology described in Example 1), was demonstrated to be 98.4% monomeric as determined by SEC (Size Exclusion Chromatography HPLC) and had a purity of >93% Main Peak by RP-HPLC.DOM30h440-81/86 showed very good biophysical characteristics when tested over a wide range of pH and ionic strength conditions. Solubility was achieved up to at least 40 mg/ml in naked buffers and 10 mg/ml in buffers containing NaCl from pH2.5 to 9.0 and NaCl concentrations ranging between 0.0 and 2.0M. Slight aggregation was noted only at extremes of pH i.e. pH2.5 and 9.0. Slight conformational changes were noted only at low pH (pH4.5 and below). DOM30h440-81/86 samples subjected for biophysical screening demonstrated very good thermal stability measured by standard Differential Scanning calorimetry (DSC) techniques (Tm ranged between 53.8 and 67.2° C.).

Formulation studies showed that DOM30h440-81/86 can be formulated as a spray-dried or a lyophilized product. These formulations were subjected to evaluation under various conditions (temperature and time) as part of a stability study. No insurmountable physical or chemical degradation or oxidation was observed in either spray dried or lyophilized formulations after 3 months of storage under different conditions.

An example spray dried formulation may be prepared from the product of the downstream purification process in FIG. 6 (45-55 mg/ml Dom30h440-81-86 in 20 mM phosphate). 9.5-11.5 g trehalose dihydrate and 6.4-7.7 g L-leucine were dissolved in water followed by addition of 1000 gTSLP binding protein solution and further dilution with water to achieve a 5% solution concentration followed by spray drying using GEA Niro SD Micro with an inlet temperature of 125° C. and an outlet temperature of 70° C., a drying gas flow of 30 kg/h and an atomising gas flow of 5 kg/h to provide the following powder:

| | Spray Dried weight concentration ranges |
|---|---|
| Material | (%)* |
| Dom30h440-81-86 | 67.3-68.0 |
| Phosphate Buffer | 3.3-4.0 |
| Trehalose Dihydrate | 14.3 |
| L-Leucine | 9.5 |

*anticipating 5% residual moisture content

An example blend may be prepared from the spray dried powder by weighing out suitable quantities of spray dried powder and DPPC/lactose carrier to create the intended blend strength and quantity, for example 30 g of spray dried powder and 20 g of DPPC/lactose added to a container. Finally the two components are blended using a Turbula System at 46 min⁻¹ for 60 minutes.

| Material | Blend concentration ranges (%) |
|---|---|
| Spray dried powder | 0.3-60 |
| DPPC/lactose | 40-99.7 |

### Example 11 Frequency of Anti-Drug Antibodies (ADA) to DOM30h-440-81/86 in Healthy Donor Sera

A specific immunoassay was used to determine the frequency of anti-drug antibodies (ADA) to DOM30h-440-81/86 in healthy donor sera.

Specific Immunoassay to Determine Binding of dAbs to Anti-Drug Antibodies
1. In a microtitre assay plate, 5% serum sample in assay diluent (1% casein in PBS) is incubated with a homogeneous mixture containing 0.1 μg/mL biotinylated test materials (e.g., DOM30h-440-81/86) and 0.2 μg/mL ruthenylated (“Sulfo-Tag”™) test materials (e.g., DOM30h-440-81/86), in assay diluent (1% casein in PBS).
2. A MSD™ streptavidin plate is blocked with 150 μl Blocking buffer (1% casein in PBS) at room temperature (RT) for 1 hour with shaking. The blocker is removed without washing.
3. The sample (50 μl) is transferred from the assay plate to the MSD™ streptavidin plate and incubated for 1 hour at RT.
4. After the 1 hour incubation, the MSD plate is then washed 3 times with PBS-Tween, read buffer (150 μL/well) is added and the plate is read using an MSD Sector Imager.
5. The luminescence signal in the assay is generated when the biotinylated and ruthenylated molecules of the test material e.g. DOM30h-440-81/86 are cross-linked by antibodies present in the serum sample.
6. The percentage positive rate was determined as the % of samples above the assay cutpoint. Assay cutpoint was defined after the removal of any outliers as the [mean luminescence of the population+1.654×standard deviation].

Example data are shown in FIG. 4. The frequency of sera from healthy subjects which gave a positive signal (above the assay cutpoint) for DOM30h-440-81/86 was 6.7%, compared with 11% for DT02-K-044-085

### Example 12: Inhalation of DOM30h-440-81/86

One male and one female cynomolgus monkey was dosed with a spray dried composition (DOM30h-440-81/86 (61.07%), phosphate buffer salts (3.94%), trehalose (30% w/w), leucine (5% w/w)) formulated at a nominal concentration of 20% in vehicle (1% w/w dipalmitoylphosphatidylcholine (DPPC) in lactose) by daily 1 hour face mask inhalation at overall estimated doses of 0, 880, 2272 or 7046 mg/kg/day for 14 days. Plasma samples were taken immediately after dosing and at 0.5, 1, 2, 5, 8, 12 and 23 hours after the end of the 1 hour inhalation period on days 1 and 14. Bronchoalveolar lavage (BAL) samples were taken from animals at necropsy by a single wash with approximately 10 ml of isotonic saline solution. 5 μl of each plasma sample (diluted 2.5 fold with deionised water) and 50 μl of each BAL sample was analysed for urea using the commercially available QuantiChrom assay. The volume of epithelial lung fluid (ELF) contained in the BAL samples were estimated by measurement of the mean endogenous urea in plasma per animal on Day 14 and terminal BAL samples to determine the ELF dilution. 50 μl of each plasma/BAL sample (diluted 5 fold in SuperBlock®T20) was also analysed for DOM30h-440-81/86 concentration, as follows:
1. 35 μl biotinylated human TSLP in SuperBlock®T20 (PBS) (4 μg/ml) was applied to each well of a 96 well small spot streptavidin plate (MesoScale Discovery). The plates were sealed and incubated for approximately 1 hour at 37° C. with shaking.
2. The plates were washed five times with 300 μl wash buffer (10 mM sodium phosphate, 150 mM NaCl, 0.1% Tween 20, pH 7.5)
3. 200 μl blocking buffer (SuperBlock®T20 (Thermo, product number 37516)+5% NHP plasma) was added to each well. The plates were sealed and incubated for approximately 1 hour at 37° C. with shaking.
4. The plates were washed five times with 300 μl wash buffer (10 mM sodium phosphate, 150 mM NaCl, 0.1% Tween 20, pH 7.5)
5. 35 μl samples (or calibration standards) were added. The plates were sealed and incubated for approximately 2 hours at 37° C. with shaking.
6. The plates were washed five times with 300 μl wash buffer (10 mM sodium phosphate, 150 mM NaCl, 0.1% Tween 20, pH 7.5)
7. 35 μl reporter tag solution (0.5 μg/ml ruthenium labelled anti-VKappa mAb in a 1:50 dilution of HBR-9 (Thermo, part number 3KC564) in SuperBlock®T20 (Thermo, product number 37516)) was added to each well. The plates were sealed and incubated for approximately 1 hour at 37° C. with shaking.
8. The plates were washed five times with 300 μl wash buffer (10 mM sodium phosphate, 150 mM NaCl, 0.1% Tween 20, pH 7.5)
9. 150 μl development substrate (0.87×MSD Read Buffer T with surfactant (MSD, catalogue number R92TC-1) was added to each well. After 1.5-2 minutes, electroluminescence was measured using a plate reader

The plasma samples were analysed against a plasma calibration line whilst BAL samples were analysed against a BAL calibration line. The ELF dilution factor was used to calculate the concentration of DOM30h-440-81/86 in the ELF contained within the BAL samples. Key plasma pharmacokinetic parameters for the male and female groups are provided in Tables 12 and 13, and the concentration of DOM30h-440-81/86 in epithelial lining fluid is summarised in Table 14.

TABLE 12

| | | Male (n = 1/group) | | |
|---|---|---|---|---|
| | | Estimated Inhaled Dose of DOM30h-440-81/86 | | |
| | | (μg/kg/day) ^{a} | | |
| Parameter | Period | 880 | 2272 | 7046 |
| AUC₀₋ₜ ^{b} | Day 1 | 1360 | 1230 | 7550 |
| (ng · h/mL) | Day 14 | 188 | 1240 | 1900 |
| Cₘₐₓ ^{b} | Day 1 | 217 | 135 | 1040 |
| (ng/mL) | Day 14 | 24.2 | 150 | 178 |
| Tₘₐₓ ^{c} | Day 1 | 3.0 | 1.5 | 6.0 |
| (h) | Day 14 | 3.0 | 2.0 | 3.0 |

TABLE 13

| | | Female (n = 1/group) | | |
|---|---|---|---|---|
| | | Estimated Inhaled Dose of DOM30h-440-81/86 | | |
| | | (μg/kg/day) ^{a} | | |
| Parameter | Period | 880 | 2272 | 7046 |
| AUC₀₋ₜ ^{b} | Day 1 | 1770 | 2230 | 6670 |
| (ng · h/mL) | Day 14 | 717 | 1050 | 4540 |
| Cₘₐₓ ^{b} | Day 1 | 269 | 341 | 831 |
| (ng/mL) | Day 14 | 97.6 | 157 | 440 |
| Tₘₐₓ ^{c} | Day 1 | 3.0 | 3.0 | 1.5 |
| (h) | Day 14 | 3.0 | 1.5 | 3.0 |

a. Overall estimated inhaled dose for the entire study
b. Calculated by dose-normalizing on the individual TK sampling occasion per sex and re-normalizing with the overall estimated inhaled dose for the entire study.
c. Tₘₐₓ values are from the start of the 1 hour inhalation period

TABLE 14

| Estimated Inhaled Dose^{a} | | Concentration of DOM30h-440- |
|---|---|---|
| (μg/kg/day) | Sex | 81/86 (ng/mL of ELF) |
| 0 | M | NC |
| | F | NC |
| 880 | M | 78.6 |
| | F | 141 |
| 2272 | M | 340 |
| | F | 260 |
| 7046 | M | 770 |
| | F | 1310 |

NC = Not calculated
^{a}Overall estimated inhaled dose

DOM30h-440-81/86 was not quantifiable in any of the plasma and BAL samples collected from the vehicle control animals. In the animals administered DOM30h-440-81/86, the concentration of DOM30h-440-81/86 in ELF generally increases with increasing dose. Systemic exposure was generally lower on day 14 than on day 1. The small sample size (n=1/sex/group) makes it difficult to conclude differences in systemic exposure between the sexes or proportionality between the doses.

### Example 13: DOM30h-440-81/86 Inhibits IL-5 and/or IL-13 Production from Human Nasal Polyp Cells Stimulated with a Mixture of TSLP, IL-33 and IL-25

Nasal polyp tissue (with appropriate consent compliant with the UK Human Tissue Act) was finely chopped into fragments which were digested with 125 μg/ml endotoxin free collagenase (Liberase™, Roche Diagnaostics) and 25 μg/ml DNase (Sigma-Aldrich) for 1 h at 37° C., with shaking, to obtain a single cell suspension. Cells were washed twice with AIM V serum-free medium (Life Technologies), and then resuspended at 4×10⁶ cells/ml in AIM V medium. DOM30h-440-81/86 was diluted to 4-times final required concentrations and 50 ul/well was added to flat-bottomed 96-well plate. 504 of AIM V medium containing 40 ng/ml each of TSLP, IL-25 and IL-33 (R&D Systems) to give a final concentration of 10 ng/ml of each cytokine was then added, followed by 100 ul of cell suspension (4×10⁵ cells per well). Plates were incubated for 96 hours at 37° C., 5% CO₂ after which supernatants were collected, and IL-5 and IL-13 were measured using Luminex multiplex assay.

The stimulatory responses observed in the nasal tissue assay were highly variable, and DOM30h-440-81/86 correspondingly showed varying degrees of efficacy depending on the donor tissue studied, ranging between no inhibition, partial inhibition and complete inhibition of IL-5 and/or IL-13.

### Example 14: Crystal Structure of DOM30h-440-81/86 Complexed to Human TSLP

The complex was made by mixing 24.6 mg of purified recombinant refolded human TSLP (from E. coli) with 20 mg recombinant DOM30h-440-81/86 (a molar ratio of 0.91 hTSLP to DOM30h-440-81/86), prior to concentration to a volume of ˜2 ml using a centrifugal concentration device fitted with a 5 k molecular weight cut off membrane (VivaSpin 20 Sarorious: catalogue no. VS2012). The complex was then purified from uncomplexed material using Superdex S75 size exclusion column (GE Healthcare 17-1180-01) equilibrated with running buffer of PBS containing 0.5M arginine. The resolved complex was again concentrated using a Vivaspin 20 before dialysed into a final buffer of 20 mM HEPES pH 7.0, 150 nM NaCl. The final yield was 53 μl of protein at 57.78 mg/mL, (as measured by absorbance at 280 nm). The complex components were validated by protein intact mass spectrometry and SDS-PAGE gel.

The human TSLP-DOM30h-440-81/86 purified complex in 20 mM HEPES pH 7.0, 150 mM NaC at 33.4 mg/ml was co-crystallised using 20% w/v PEG 3350 and 0.02M Na K Phosphate as a precipant in sitting drops consisting of 1:1 ratio of well to protein solution. Crystals were cryoprotected using cryoprotectant consisting of well solution with 20% PEG200 and paratone prior to flash freezing in liquid nitrogen. Data from a single crystal was collected at the Europeon Synchrotron Radiation Facility (Genoble) and processed to 1.84 Å using XDS (Kabsch, W. (2010) XDS. Acta Cryst. D66, 125-132.) and AIMLESS (Evans, P. R., et al. (2013) Acta Cryst. D69, 1204-1214) within AUTOPROC (Vonrhein, C., et al. (2011) Acta Cryst. D67, 293-302.). A molecular replacement solution containing 4 complexes in the ASU was determined using PHASER (J. Appl. Cryst. (2007). 40, 658-674) within PHENIX (Adams P D, (2010) Acta Cryst. D66, 213-221). Iterative manual model building was performed using COOT (Emsley, P., et al. (2004) Acta Cryst., D60, 2126-2132) and refined using PHENIX. The human TSLP-DOM30h-440-81/86 interaction interface was well defined and consistent in all four complexes within the ASU.

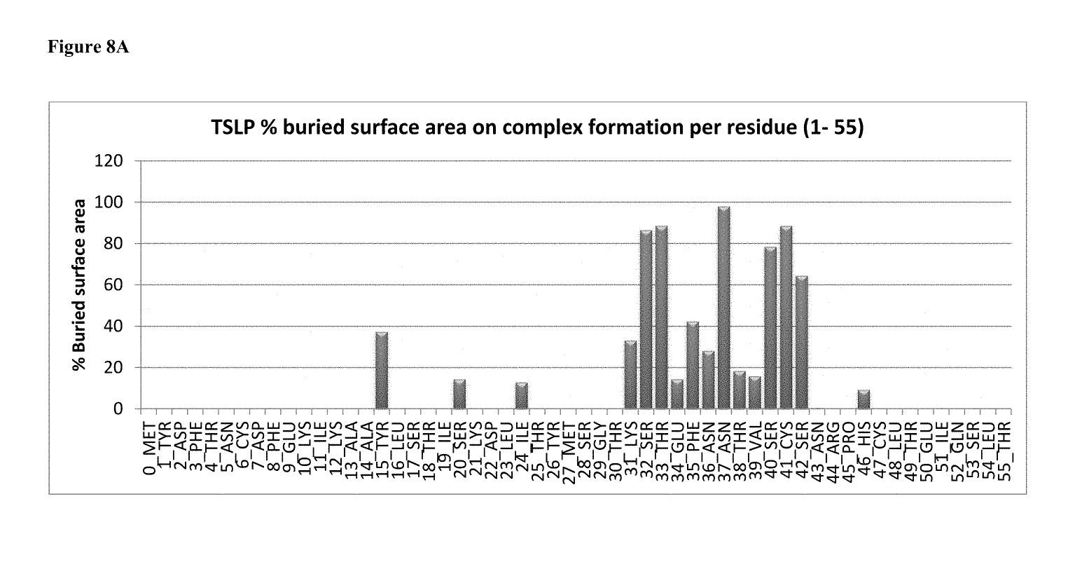

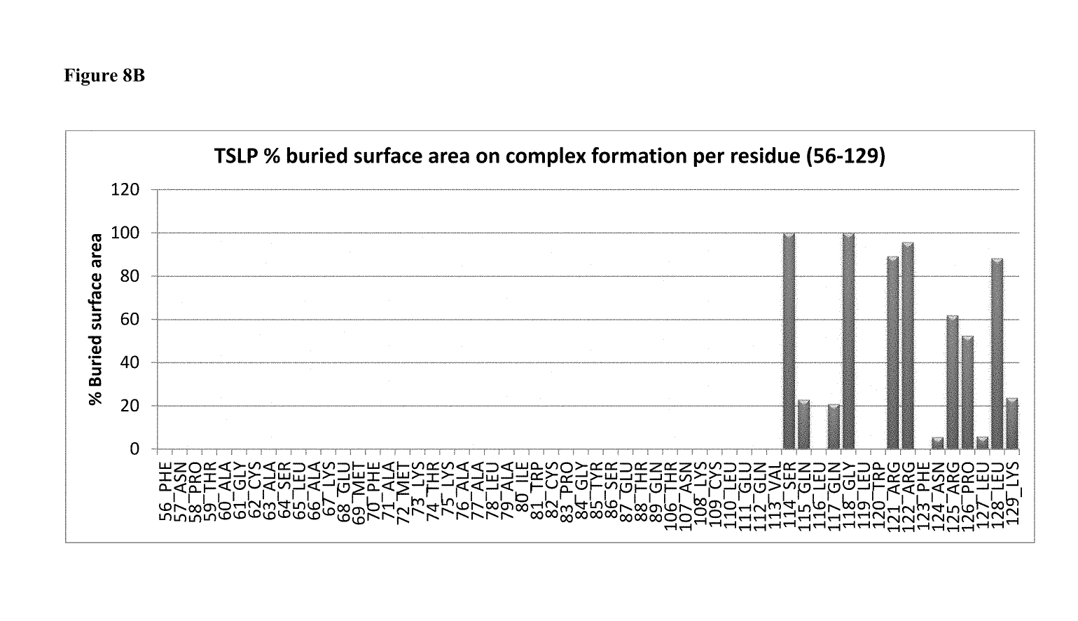

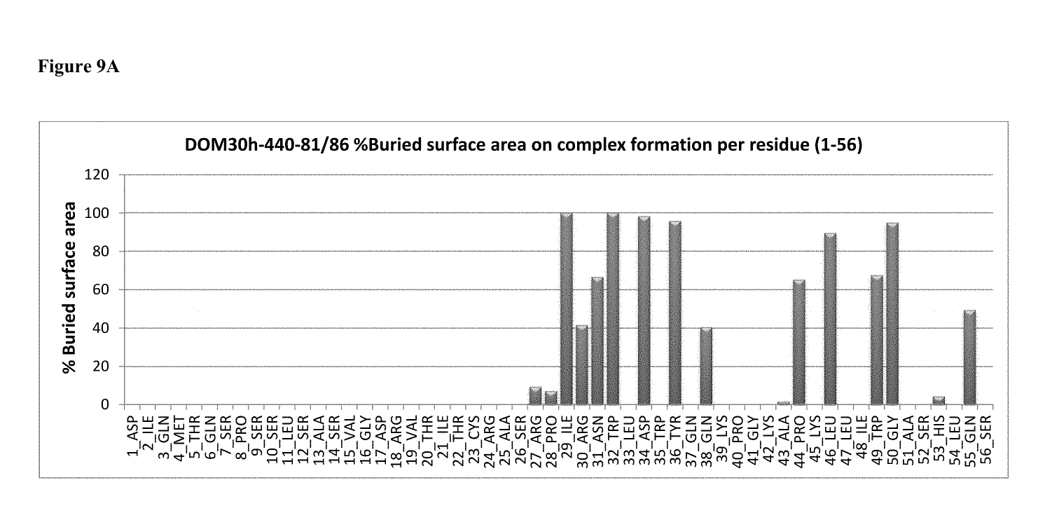

The structure of the hTSLP-DOM30h-440-81/86 complex can be overlaid on the structure of the rat TSLP/ILRa/TSLP receptor complex (FIG. 8). This shows that DOM30h-440-81/86 directly interferes with the interaction between TSLP and the TSLPR. As discussed above, this is highly desirable since TSLP antagonists that act by preventing recruitment of the IL-7Rα chain, or by binding directly to TSLPR (or IL-7Rα), may be internalised and processed as antigens more effectively than a TSLP antagonist that binds TSLP and stays in solution as a complex with TSLP.

The epitope for DOM30h-440-81/86 can be defined more precisely by identifying residues on human TSLP that become inaccessible to solvent on binding to DOM30h-440-81/86. Accordingly, the anti-TSLP dAb/human TSLP co-crystal structure was analysed using Qt-PISA v2.0.1 (Protein Interfaces, Complexes and Assemblies; Krissinel and Henrick (2007) and the buried surface area (BSA) was calculated for each residue of the human TSLP. FIG. 8 shows the % exposed surface area of each residue of TSLP that becomes buried on binding to DOM30h-440-81/86. Those residues on human TSLP that become buried on binding to DOM30h-440-81/86 represent the epitope. Based on this, the epitope for DOM30h-440-81/86 on human TSLP includes the following residues: Tyr15, Ser20, Ile24, Lys31, Ser32, Thr33, Glu34, Phe35, Asn36, Asn37, Thr38, Val39, Ser40, Cys41, Ser42, Asn43, His46, Ser114, Gln115, Gln117, Gly118, Arg121, Arg122, Asn124, Arg125, Pro126, Leu127, Leu128 and Lys129.

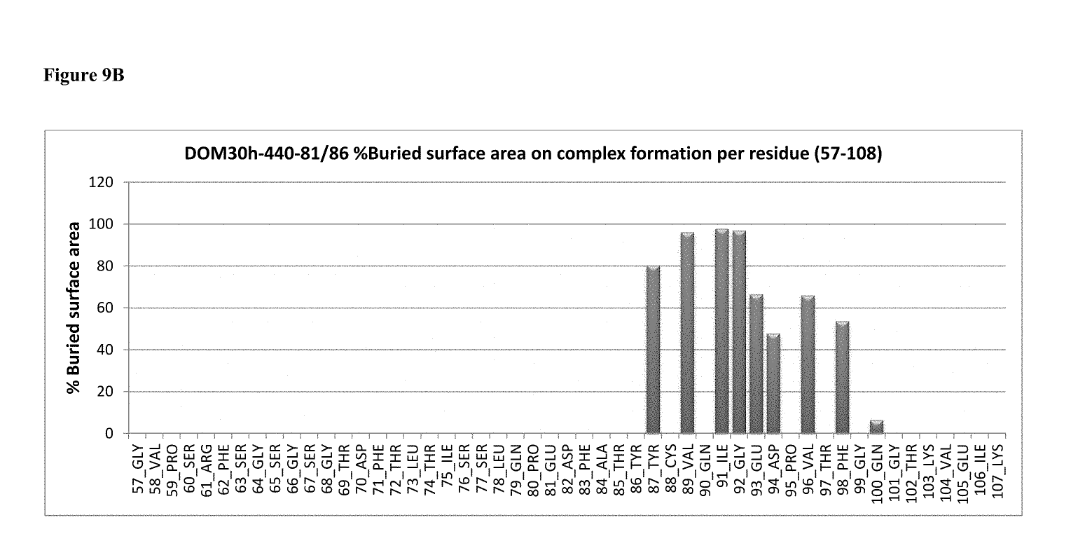

The portion of an antibody or fragment thereof that binds an epitope is termed a paratope. CDRs are widely accepted as being the key regions of the paratope, but other residues may also be important. Accordingly, the paratope of DOM30h-440-81/86 was identified by identifying residues on DOM30h-440-81/86 that become inaccessible to solvent on binding to human TSLP (using the same techniques used to identify the epitope of DOM30h-440-81/86). FIG. 9 shows the % exposed surface area of each residue of DOM30h-440-81/86 that becomes buried on binding to human full length TSLP. Based on this, the paratope of DOM30h-440-81/86 includes the following residues: Arg27, Pro28, Ile29, Arg30, Asn31, Trp32, Asp34, Tyr36, Gln38, Pro44, Leu46, Trp49, Gly50, His53, Gln55, Tyr87, Val89, Ile91, Gly92, Glu93, Asp94, Val96, Phe98 and Gln100. Unsurprisingly, two thirds of these residues fall within the CDR regions (defined according to the Kabat numbering scheme), namely Arg27, Pro28, Ile29, Arg30, Asn31, Trp32 and Asp34 of CDRL1, Gly50, His53 and Gln55 of CDRL2, and Val89, Ile91, Gly92, Glu93, Asp94 and Val96 of CDRL3. It is also noted that, should CDRL2 be defined according to the Contact numbering system, Leu 46 and Trp49 would also be considered to be CDR residues.

A protein multiple sequence alignment of DOM30h-440-81/86 against the Vk/Jk germlines was conducted. The framework residues Tyr36, Gln38, Pro44, Leu46 (considered a CDR residue, if the Contact numbering system is used), Tyr87, Phe98 and Gln100 are conserved at 60% or greater identity across the functional human Vk and Jk genes. This level of conservation suggests that these residues have a structural role. However, the fact that other residues are seen in functional human Vk and Jk genes suggests variations may be tolerated. In contrast, Trp49 (considered a CDR residue if the Contact numbering system is used) is unique amongst the genes compared suggesting that this may need to be preserved.

The interactions between the epitope and paratope were defined using CCG (Chemical Computing Group) MOE v2014.09 (Molecular Operating Environment). Protein residues within 7 Å of the dAb or TSLP were selected, and then the “Ligand Interaction” tool with the default parameters was used to identify water molecules or residues from the interacting molecule that were deemed to be interacting with these residues. Note that due to this tool being designed for defining small molecule ligand interactions, rather than protein residues, the “Ligand interactions” of each selected residue were calculated individually. The interactions defined by MOE were edited to delete any intrachain interactions, and to delete all water interactions apart from those that formed a bridge between the two chains. The remaining interacting residues are shown below:

### Epitope

Direct interaction with anti-TSLP dAb residues only: Lys31, Phe35, Arg121, Arg122
Direct interaction with anti-TSLP dAb residues, and indirect interaction via water: Ser32, Thr33, Asn37
Indirect interaction via water only: Tyr15, Asn36, Ser40, Cys41, Ser42, Leu128

### Paratope

Direct interaction with TSLP residues only: Trp32, Ile91
Direct interaction with TSLP residues, and indirect interaction via water: Arg30, Asn31, Asp34, Glu93, Asp94
Indirect interaction via water only: Pro28, Tyr36, Gln38, Pro44, Leu46, Gly50, His53, Gln55, Ser67, Gly92

The interacting residues in the epitope are all residues that become more inaccessible to solvent upon binding DOM-30h-440-81/86. Ser67 is a residue on DOM-30h-440-81/86 that did not become more inaccessible to solvent upon binding TSLP, but does however interact with TSLP via water. Ser67, like a number of other framework residues, is conserved at greater than 60% identity across the functional human Vk and Jk genes, suggesting that this residue may have a structural role.

### Example 15: Hydrogen-Deuterium Exchange of DOM30h-440-81/86 Complexed to Human TSLP

Protein solutions of full length human TSLP and DOM30h-440-81/86 alone, as well as TSLP-DOM30h-440-81/86 complex were initially diluted into non-deuterated H₂O buffers to give working stock concentration in excess of 10 μM. A typical buffer would be 10 mM phosphate H2O pH7.4.

The HDX labeling reaction was carried out with a 10-20 fold dilution from non-deuterated into deuterated buffer at 20-23° C. Samples were taken at 4-5 given time points between 0-1800 seconds (e.g. 0, 120, 300 and 1800, or 0, 30, 100, 300 and 1000). The samples were quenched with a pre-chilled low pH and denaturing quench buffer at 4° C., consisting of either 200 mM sodium phosphate, 4 M guanidine HCl, 200 mM tris(2-carboxyethyl)phosphine, pH 2.2 or 7M urea, 0.8 M tris(2-carboxyethyl)phosphine, 0.4 M guanidine HCl, 0.1% formic acid. Between 1-4 replicates were taken for each timepoint.

The denatured quenched samples were injected onto an immobilised pepsin digestion column (Waters Enzymate BEH, 2.1 mm×30 mm, Part no: 186007233) with a digestion time of typically 180-240 seconds, a column flow rate of 70 μl/min and digest buffer of 0.2% aqueous formic acid at 20° C. The released peptides were analysed by a UPLC-MS at 0-5° C. For example on a Acquity UPLC system using a 1.0×100 mm UPLC BEH C18 column (Part no 186002346) with a 17 minute run, mobile phase A: H2O/formic acid (99.8:0.2 v/v) and mobile phase B: acetonitrile/formic acid (99.8:0.2 v/v) at a flow rate of 40 μl/min.

Peptides produced by proteolysis of the non-deuterated proteins were identified using a ProteinLynxGlobal SERVER (PLGS™; http://www.waters.com/waters/en_GB/ProteinLynx-Global-SERVER%28PLGS%29/nav.htm?cid=513821&locale=en_GB), or a Mascot search engine, against a database containing only the protein sequences of interest. The deuterium incorporation for each time point and state was calculated using DynamX Analysis Software v 3.0 (or HD Examiner) by comparing the undeuterated peptide list imported from PLGS to the data acquired for the deuterated samples.

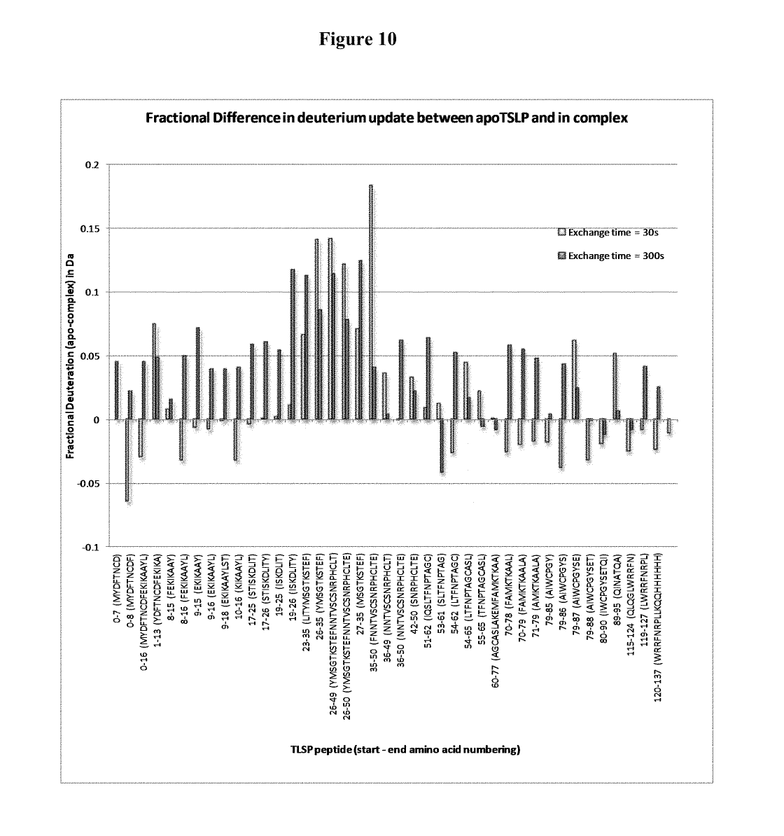

The regions Tyr26 to Ser42 and Gln89 to Ala95 show the most significant changes on DOM30h-440-81/86 binding. This indicates suggests a conformational epitope where the two linear peptide regions are brought together by the three dimensional structure of the antigen. Examining the underlying pepsin peptide data, multiple peptides containing all or portions of the most N-terminal region Tyr26-Ser42 consistently exhibit slower exchange and thus protection from deuteration on complex formation. This can easily be seen in the representative summary plot in FIG. 3. This plot summarises the HDX-MS peptide data by plotting the difference in deuteration between complexed and complexed TSLP peptides, at defined time points (t=30 s, 300 s) after dilution into deuterated buffer. A positive difference in deuterium update between uncomplexed TLSP and in complex with DOM30h-440-81/86 in FIG. 10 shows enhanced protection of TLSP from solvent exchange on complexation. A threshold of 0.5 Da is often used to indicate a signficant difference in update.

Sequence Listing

SEQ ID NO: 1
CDRL1 of Dom30h-440-81/86, Dom30h-440-53, Dom30h-
440-54, Dom30h-440-55, Dom30h-440-56, Dom30h-440-
57, Dom30h-440-58, Dom30h-440-60, Dom30h-440-63,
Dom30h-440-64 and Dom30h-440-65 (Kabat, Chothia
and AbM CDR definition)
RASRPIRNWLD
SEQ ID NO: 2
CDRL1 of Dom30h-440-81/86 (Contact CDR definition)
RNWLDWY
SEQ ID NO: 3
CDRL1 of Dom30h-440-81/86 (minimum binding unit)
RNWLD
SEQ ID NO: 4
CDRL2 of Dom30h-440-81/86, Dom30h-440-53, Dom30h-
440-54, Dom30h-440-55, Dom30h-440-56, Dom30h-440-
57, Dom30h-440-58, Dom30h-440-60, Dom30h-440-63,
Dom30h-440-64 and Dom30h-440-65 (Kabat, Chothia,
AbM CDR definition)
GASHLQS
SEQ ID NO: 5
CDRL2 of Dom30h-440-81/86 (Contact CDR definition)
LLIWGASHLQ
SEQ ID NO: 6
CDRL2 of Dom30h-440-81/86 (minimum binding unit)
GASHLQ
SEQ ID NO: 7
CDRL3 of Dom30h-440-81/86 and Dom30h-440-55
(Kabat, Chothia, AbM CDR definition)
VQIGEDPVT
SEQ ID NO: 8
CDRL3 of Dom30h-440-81/86 (Contact CDR and minimum
binding unit definition)
VQIGEDPV
SEQ ID NO: 9
Dom30h-440-81/86 amino acid sequence
DIQMTQSPSSLSASVGDRVTITCRASRPIRNWLDWYQQKPGKAPKLLIWG
ASHLQSGVPSRFSGSGSGTDFTLTISSLQPEDFATYYCVQIGEDPVTFGQ
GTKVEIK
SEQ ID NO: 10
Non-codon optimised DNA sequence (Dom30h-440-81)
GACATCCAGATGACCCAGTCTCCATCCTCCCTGTCTGCATCTGTAGGAGA
CCGTGTCACCATCACTTGCCGGGCAAGTCGGCCCATTCGGAATTGGTTAG
ATTGGTACCAGCAGAAACCAGGGAAAGCCCCTAAGCTCCTGATCTGGGGG
GCGTCCCACTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGTGCAGATTGGGGAGGATCCTGTGACGTTCGGCCAA
GGGACCAAGGTGGAAATCAAA
SEQ ID NO: 11
Codon-optimised DNA sequence (Dom30h-440-86)
GATATCCAGATGACCCAGTCTCCGTCTTCCCTGTCTGCGTCCGTTGGTGA
CCGTGTAACCATCACTTGTCGTGCAAGCCGTCCGATCCGTAACTGGCTGG
ATTGGTACCAGCAGAAACCGGGTAAAGCGCCGAAACTGCTGATCTGGGGT
GCTTCTCACCTGCAGTCTGGTGTTCCGTCCCGTTTCTCTGGCTCTGGTAG
CGGTACCGACTTCACCCTGACTATCTCTAGCCTGCAGCCGGAAGACTTCG
CGACCTACTACTGCGTTCAGATCGGTGAAGACCCGGTTACCTTCGGTCAG
GGCACCAAAGTAGAAATCAAA
SEQ ID NO: 12
Dom30h-440-87/93 amino acid sequence
DIQMTQSPSSLSASVGDRVTITCRASRPIRNWLDWYQQKPGKAPELLIWG
ASHLQSGVPSRFSGSGSGTDFTLTISSLQPEDFATYYCVQIGEDPVTFGQ
GTKVEIK
SEQ ID NO: 13
Non-codon optimised DNA sequence (Dom30h-440-87)
GATATCCAGATGACCCAGTCTCCGTCTTCCCTGTCTGCGTCCGTTGGTGA
CCGTGTAACCATCACTTGTCGTGCAAGCCGTCCGATCCGTAACTGGCTGG
ATTGGTACCAGCAGAAACCGGGTAAAGCGCCGGAACTGCTGATCTGGGGT
GCTTCTCACCTGCAGTCTGGTGTTCCGTCCCGTTTCTCTGGCTCTGGTAG
CGGTACCGACTTCACCCTGACTATCTCTAGCCTGCAGCCGGAAGACTTCG
CGACCTACTACTGCGTTCAGATCGGTGAAGACCCGGTTACCTTCGGTCAG
GGCACCAAAGTAGAAATCAAA
SEQ ID NO: 14
Codon-optimised DNA sequence (Dom30h-440-93)
GACATCCAGATGACCCAGTCTCCATCCTCCCTGTCTGCATCTGTAGGAGA
CCGTGTCACCATCACTTGCCGGGCAAGTCGGCCCATTCGGAATTGGTTAG
ATTGGTACCAGCAGAAACCAGGGAAAGCCCCTGAGCTCCTGATCTGGGGG
GCGTCCCACTTGCAAAGTGGGGTCCCATCACGTTTCAGTGGCAGTGGATC
TGGGACAGATTTCACTCTCACCATCAGCAGTCTGCAACCTGAAGATTTTG
CTACGTACTACTGTGTGCAGATTGGGGAGGATCCTGTGACGTTCGGCCAA
GGGACCAAGGTGGAAATCAAA
SEQ ID NO: 15
CDRL3 of Dom30h-440-53 (Kabat definition)
LQVGEDPVT
SEQ ID NO: 16
CDRL3 of Dom30h-440-54 (Kabat definition)
WQLAFDPTT
SEQ ID NO: 17
CDRL3 of Dom30h-440-56 and Dom30h-440-65 (Kabat
definition)
MQIGEDPVT
SEQ ID NO: 18
CDRL3 of Dom30h-440-57 (Kabat definition)
MQIGDDPVT
SEQ ID NO: 19
CDRL3 of Dom30h-440-58 (Kabat definition)
LQIADDPVT
SEQ ID NO: 20
CDRL3 of Dom30h-440-60 (Kabat definition)
IQFGEDPVT
SEQ ID NO: 21
CDRL3 of Dom30h-440-63 (Kabat definition)
MQIGSDPVT
SEQ ID NO: 22
CDRL3 of Dom30h-440-64 (Kabat definition)
LQIGEDPVT
SEQ ID NO: 23
Dom30h-440-53 amino acid sequence
DIQMTQSPSSLSASVGDRVTITCRASRPIRNWLDWYQQKPGKAPKLLIWG
ASHLQSGVPSRFSGSGSGTDFTLTISSLQPEDFATYYCLQVGEDPVTFGQ
GTKVEIKR
SEQ ID NO: 24
Dom30h-440-54 amino acid sequence
DIQMTQSPSSLSASVGDRVTITCRASRPIRNWLDWYQQKPGKAPKLLIWG
ASHLQSGVPSRFSGSGSGTDFTLTISSLQPEDFATYYCWQLAFDPTTFGQ
GTKVEIKR
SEQ ID NO: 25
Dom30h-440-55 amino acid sequence
DIQMTQSPSSLSASVGDRVTITCRASRPIRNWLDWYQQKPGKAPKLLIWG
ASHLQSGVPSRFSGSGSGTDFTLTISSLQPEDFATYYCVQIGEDPVTFGQ
GTKVEIKR
SEQ ID NO: 26
Dom30h-440-56 amino acid sequence
DIQMTQSPSSLSASVGDRVTITCRASRPIRNWLDWYQQKPGKAPKLLIWG
ASHLQSGVPSRFSGSGSGTDFTLTVSSLQPEDFATYYCMQIGEDPVTFGQ
GTKVEIKR
SEQ ID NO: 27
Dom30h-440-57 amino acid sequence
DIQMTQSPSSLSASVGDRVTITCRASRPIRNWLDWYQQKPGKAPELLIWG
ASHLQSGVPSRFSGSGSGTDFTLTISSLQPEDFATYYCMQIGDDPVTFGQ
GTKVEIKR
SEQ ID NO: 28
Dom30h-440-58 amino acid sequence
DIQMTQSPSSLSASVGDRVTITCRASRPIRNWLDWYQQKPGKAPKLLIWG
ASHLQSGVPSRFSGSGSGTDFTLTISSLQPEDFATYYCLQIADDPVTFGQ
GTKVEIKR
SEQ ID NO: 29
Dom30h-440-60 amino acid sequence
DIQMTQSPSSLSASVGDRVTITCRASRPIRNWLDWYQQKPGKAPKLLIWG
ASHLQSGVPSRFSGSGSGTDFTLTISSLQPEDFATYYCIQFGEDPVTFGQ
GTKVEIKR
SEQ ID NO: 30
Dom30h-440-63 amino acid sequence
DIQMTQSPSSLSASVGDRVTITCRASRPIRNWLDWYQQKPGKAPKLLIWG
ASHLQSGVPSRFSGSGSGTDFTLTISSLQPEDFATYYCMQIGSDPVTFGQ
GTKVEIKR
SEQ ID NO: 31
Dom30h-440-64 amino acid sequence
DIQMTQSPSSLSASVGDRVTITCRASRPIRNWLDWYQQKPGKAPKLLIWG
ASHLQSGVPSRFSGSGSGTDFTLTISSLQPEDFATYYCLQIGEDPVTFGQ
GTKVEIKR
SEQ ID NO: 32
Dom30h-440-65 amino acid sequence
DIQMTQSPSSLSASVGDRVTITCRASRPIRNWLDWYQQKPGKAPKLLIWG
ASHLQSGVPSRFSGSGSGTDFTLTISSLQPEDFATYYCMQIGEDPVTFGQ
GTKVEIKR
SEQ ID NO: 33
Dom30h-440-88/92 amino acid sequence
DIQMTQSPSSLSASVGDRVTITCRASRPIRNWLDWYQQKPGKAPELLIWG
ASHLQSGVPSRFSGSGSGTDFTLTISSLQPEDFATYYCMQIGDDPVTFGQ
GTKVEIK
SEQ ID NO: 34
Dom30h-440-89/94 amino acid sequence
DIQMTQSPSSLSASVGDRVTITCRASRPIRNWLDWYQQKPGKAPKLLIWG
ASHLQSGVPSRFSGSGSGTDFTLTISSLQPEDFATYYCVQIGEDPVTFGQ
GTKVEIKR
SEQ ID NO: 35
Dom30h-440-90/95 amino acid sequence
DIQMTQSPSSLSASVGDRVTITCRASRPIRNWLDWYQQKPGKAPELLIWG
ASHLQSGVPSRFSGSGSGTDFTLTISSLQPEDFATYYCVQIGEDPVTFGQ
GTKVEIKR
SEQ ID NO: 36
Dom30h-440-91/96 amino acid sequence
DIQMTQSPSSLSASVGDRVTITCRASRPIRNWLDWYQQKPGKAPELLIWG
ASHLQSGVPSRFSGSGSGTDFTLTISSLQPEDFATYYCMQIGDDPVTFGQ
GTKVEIKR
SEQ ID NO: 37
Peptide sequence containing long “epitope” for
TSLP binding protein
YMSGTKSTEFNNTVSCS
SEQ ID NO: 38
Peptide sequence containing short “epitope” for
TSLP binding protein
KSTEFNNTVSCS
SEQ ID NO: 39
Peptide containing residues 0-7 of full length
human TSLP
MYDFTNCD
SEQ ID NO: 40
Peptide containing residues 0-8 of full length
human TSLP
MYDFTNCDF
SEQ ID NO: 41
Peptide containing residues 0-16 of full length
human TSLP
MYDFTNCDFEKIKAAYL
SEQ ID NO: 42
Peptide containing residues 1-13 of full length
human TSLP
YDFTNCDFEKIKA
SEQ ID NO: 43
Peptide containing residues 8-15 of full length
human TSLP
FEKIKAAY
SEQ ID NO: 44
Peptide containing residues 8-16 of full length
human TSLP
FEKIKAAYL
SEQ ID NO: 45
Peptide containing residues 9-15 of full length
human TSLP
EKIKAAY
SEQ ID NO: 46
Peptide containing residues 9-16 of full length
human TSLP
EKIKAAYL
SEQ ID NO: 47
Peptide containing residues 9-18 of full length
human TSLP
EKIKAAYLST
SEQ ID NO: 48
Peptide containing residues 10-16 of full length
human TSLP
KIKAAYL
SEQ ID NO: 49
Peptide containing residues 17-25 of full length
human TSLP
STISKDLIT
SEQ ID NO: 50
Peptide containing residues 17-26 of full length
human TSLP
STISKDLITY
SEQ ID NO: 51
Peptide containing residues 19-25 of full length
human TSLP
ISKDLIT
SEQ ID NO: 52
Peptide containing residues 19-26 of full length
human TSLP
ISKDLITY
SEQ ID NO: 53
Peptide containing residues 23-35 of full length
human TSLP
LITYMSGTKSTEF
SEQ ID NO: 54
Peptide containing residues 26-35 of full length
human TSLP
YMSGTKSTEF
SEQ ID NO: 55
Peptide containing residues 26-49 of full length
human TSLP
YMSGTKSTEFNNTVSCSNRPHCLT
SEQ ID NO: 56
Peptide containing residues 26-50 of full length
human TSLP
YMSGTKSTEFNNTVSCSNRPHCLTE
SEQ ID NO: 57
Peptide containing residues 27-35 of full length
human TSLP
MSGTKSTEF
SEQ ID NO: 58
Peptide containing residues 35-50 of full length
human TSLP
FNNTVSCSNRPHCLTE
SEQ ID NO: 59
Peptide containing residues 36-49 of full length
human TSLP
NNTVSCSNRPHCLT
SEQ ID NO: 60
Peptide containing residues 36-50 of full length
human TSLP
NNTVSCSNRPHCLTE
SEQ ID NO: 61
Peptide containing residues 42-50 of full length
human TSLP
SNRPHCLTE
SEQ ID NO: 62
Peptide containing residues 51-62 of full length
human TSLP
IQSLTFNPTAGC
SEQ ID NO: 63
Peptide containing residues 53-61 of full length
human TSLP
SLTFNPTAG
SEQ ID NO: 64
Peptide containing residues 54-62 of full length
human TSLP
LTFNPTAGC
SEQ ID NO: 65
Peptide containing residues 54-65 of full length
human TSLP
LTFNPTAGCASL
SEQ ID NO: 66
Peptide containing residues 55-65 of full length
human TSLP
TFNPTAGCASL
SEQ ID NO: 67
Peptide containing residues 60-77 of full length
human TSLP
AGCASLAKEMFAMKTKAA
SEQ ID NO: 68
Peptide containing residues 70-78 of full length
human TSLP
FAMKTKAAL
SEQ ID NO: 69
Peptide containing residues 70-79 of full length
human TSLP
FAMKTKAALA
SEQ ID NO: 70
Peptide containing residues 71-79 of full length
human TSLP
AMKTKAALA
SEQ ID NO: 71
Peptide containing residues 79-85 of full length
human TSLP
AIWCPGY
SEQ ID NO: 72
Peptide containing residues 79-86 of full length
human TSLP
AIWCPGYS
SEQ ID NO: 73
Peptide containing residues 79-87 of full length
human TSLP
AIWCPGYSE
SEQ ID NO: 74
Peptide containing residues 79-88 of full length
human TSLP
AIWCPGYSET
SEQ ID NO: 75
Peptide containing residues 80-90 of full length
human TSLP
IWCPGYSETQI
SEQ ID NO: 76
Peptide containing residues 89-95 of full length
human TSLP
QINATQA
SEQ ID NO: 77
Peptide containing residues 115-124 of full length
human TSLP
QLQGLWRRFN
SEQ ID NO: 78
Peptide containing residues 119-127 of full length
human TSLP
LWRRFNRPL
SEQ ID NO: 79
Peptide containing residues 120-137 of full length
human TSLP
WRRFNRPLLKQQHHHHHH
SEQ ID NO: 80
Full length (mature) human TSLP
YDFTNCDFEKIKAAYLSTISKDLITYMSGTKSTEFNNTVSCSNRPHCLTE
IQSLTFNPTAGCASLAKEMFAMKTKAALAIWCPGYSETQINATQAMKKRR
KRKVTTNKCLEQVSQLQGLWRRFNRPLLKQQ
SEQ ID NO: 81
Short isoform human TSLP
MFAMKTKAALAIWCPGYSETQINATQAMKKRRKRKVTTNKCLEQVSQLQG
LWRRFNRPLLKQQ
SEQ ID NO: 82
Full length (mature) Cynomolgus monkey TSLP
YDFTNCDFEKIKAAYLSTISKDLITYMSGTKSTEFNNTVSCSNRPHCLTE
IQSLTFNPTAGCASLAKEMFAMKTKAALAIWCPGYSETQINATQAMKKRR
KRKVTTNKCLEQVSQLQGLWRRFNRPLLKQQ
SEQ ID NO: 83
Mature processed human IL-7
DCDIEGKDGKQYESVLMVSIDQLLDSMKEIGSNCLNNEFNFFKRHICDAN
KEGMFLFRAARKLRQFLKMNSTGDFDLHLLKVSEGTTILLNCTGQVKGRK
PAALGEAQPTKSLEENKSLKEQKKLNDLCFLKRLLQEIKTCWNKILMGTK
EH

1. A TSLP binding protein that either:
a) interacts with the following residues of full length human TSLP (SEQ ID NO: 80): Lys31 and Phe35; or
b) increases the % buried surface area of residues Ser32, Thr33, Asn37, Ser40, Cys41 and Ser42, when complexed with full length human TSLP, as compared to uncomplexed full length human TSLP; or
c) binds to full length human TSLP having at least one residue selected from: Lys31, Ser32, Thr33, Phe35, Asn36, Asn37, Ser40, Cys41, Ser42, Arg121 and Arg122 mutated exhibiting altered affinity in comparison with full length human TSLP with no mutations; or
d) binds to a peptide having the amino acid sequence set forth in SEQ ID NO: 37; or
e) binds to full length human TSLP and results in peptides derived from full length human TSLP containing part or the whole of the sequence from Tyr26 and Ser42 being more resistant to deuterium incorporation compared to corresponding peptides derived from uncomplexed full length human TSLP.

2. A TSLP binding protein as claimed in claim 1 that interacts with the following residues of full length human TSLP (SEQ ID NO: 80): Lys31 and Phe35.

3. A TSLP binding protein as claimed in claim 1 that increases the % buried surface area of residues Ser32, Thr33, Asn37, Ser40, Cys41 and Ser42 when complexed with full length human TSLP (SEQ ID NO: 80), as compared to uncomplexed full length human TSLP.

4. A TSLP binding protein as claimed in claim 1 that binds to full length human TSLP (SEQ ID NO: 80) having at least one residue chosen from: Lys31, Ser32, Thr33, Phe35, Asn36, Asn37, Ser40, Cys41, Ser42 Arg121 and Arg122 mutated exhibiting altered affinity in comparison with full length human TSLP with no mutations.

5. A TSLP binding protein as claimed in claim 1 that binds to a peptide having the amino acid sequence set forth in SEQ ID NO: 37.

6. A TSLP binding protein as claimed in claim 1 that binds to full length human TSLP (SEQ ID NO: 80) and results in peptides derived from full length human TSLP containing part or the whole of the sequence from Tyr26 and Ser42 being more resistant to deuterium incorporation compared to corresponding peptides derived from uncomplexed full length human TSLP

7. A TSLP binding protein as claimed in claim 1 that is chosen from: an antibody, a protein having an alternative antibody format, a fragment of an antibody, and a fragment of a protein having an alternative antibody format.

8. A TSLP binding protein as claimed in claim 1, which exhibits no significant binding to IL-7 (SEQ ID NO: 83).

9. A TSLP binding protein as claimed in claim 1 which exhibits cross reactivity with cynomolgus TSLP (SEQ ID NO:82).

10. A TSLP binding protein as claimed in claim 1 which exhibits no significant binding to the short isoform of human TSLP (SEQ ID NO: 81).

11. A TSLP binding protein as claimed in claim 1, wherein said TSLP binding protein does not have the amino acid sequence set forth in SEQ ID NO: 9.

12. A TSLP binding protein as claimed in claim 1, wherein said TSLP binding protein does not have the amino acid sequence set forth in any one of SEQ ID NOs: 12, 23, 24, 25, 26, 27, 28, 29, 30, 31, 32, 33, 34, 35 or 36.

13. A TSLP binding protein as claimed in claim 1, wherein said TSLP binding protein is not a TSLP-binding protein comprising:
a. CDR1, CDR2 and CDR3 of SEQ ID NO: 9 or a variant of any one or all of these CDRs, wherein the CDR variant has 1, 2, or 3 amino acid modifications; or
b. an amino acid sequence at least 90% identical to the sequence of SEQ ID NO: 9;
which TSLP binding protein has an IC50 of less than or equal to 5 nM.

14. A TSLP binding protein as claimed in claim 1, wherein said TSLP binding protein is not a TSLP binding protein that binds an epitope comprising the following residues of full length human TSLP (SEQ ID NO: 80): Tyr15, Lys31, Ser32, Thr33, Phe35, Asn36, Asn37, Ser40, Cys41, Ser42, Ser114, Gln115, Gln117, Gly118, Arg121, Arg122, Arg125, Pro126, Leu128 and Lys 129.

15. A TSLP binding protein as claimed in claim 1, wherein said TSLP binding protein does not compete for binding to TSLP with a single variable domain of SEQ ID NO:9.

16. A method of treating a disease associated with TSLP signalling in a human patient in need thereof, the method comprising administering the TSLP binding protein as claimed in claim 1 to the human patient.

17. The method of treatment as claimed in claim 16, wherein the disease associated with TSLP signalling is selected from: asthma, idiopathic pulmonary fibrosis, atopic dermatitis, allergic conjunctivitis, allergic rhinitis, Netherton syndrome, eosinophilic esophagitis (EoE), food allergy, allergic diarrhoea, eosinophilic gastroenteritis, allergic bronchopulmonary aspergillosis (ABPA), allergic fungal sinusitis, cancer, rheumatoid arthritis, COPD, systemic sclerosis, keloids, ulcerative colitis, chronic rhinosinusitis (CRS), nasal polyposis, chronic eosinophilic pneumonia, eosinophilic bronchitis, coeliac disease, Churg-Strauss syndrome, eosinophilic myalgia syndrome, hypereosinophilic syndrome, eosinophilic granulomatosis with polyangiitis and inflammatory bowel disease.

18. The method as claimed in claim 16, wherein the disease associated with TSLP signalling is asthma.

19. The method as claimed in claim 16, wherein the disease associated with TSLP signalling is severe asthma.

20. The method as claimed in claim 16, wherein the disease associated with TSLP signalling is mild to moderate asthma.

21. A pharmaceutical composition comprising the TSLP binding protein as claimed in claim 1, and optionally, at least one pharmaceutically acceptable carrier.
